(19) United States
(12) Patent Application Publication
(10) Pub. No.: US 2009/0060873 A1
(43) Pub. Date: Mar. 5, 2009

(54) Novel synthetic triterpenoids and methods of use in the treatment and prevention of multiple scleroris

(75) Inventors: Michael B. Sporn, Tunbridge, VT (US); Karen T. Liby, West Lebanon, NH (US); Gordon W. Gribble, Lebanon, NH (US); Tadashi Honda, Hanover, NH (US); John Letterio, Concord, OH (US)

Correspondence Address: FULBRIGHT & JAWORSKI L.L.P., 600 CONGRESS AVE., SUITE 2400, AUSTIN, TX, 78701, US

(73) Assignee: Reata Pharmaceuticals, Inc., Irving, TX (US)

(21) Appl. No.: 12/151,425

(22) Filed: May 5, 2008

Related U.S. Application Data
(60) Provisional application No. 60/916,273, filed on May 4, 2007.

(51) Int. Cl. A61K 38/21; C07C 233/64; A61K 31/166; A61K 31/513; A61K 39/395; A61K 31/7028; A61K 31/337; A61P 25/28; A61P 35/00; A61K 31/519; A61K 31/437; A61K 31/403; A61K 31/366; A61K 31/60

(52) U.S. Cl. 424/85.6; 564/169; 514/621; 514/274; 424/145.1; 514/27; 514/449; 514/262.1; 514/285; 514/411; 514/460; 514/165

(57) ABSTRACT

The present invention overcomes limitations of the prior art by providing new compounds and methods for the treatment of conditions, such as neurodegenerative diseases (e.g., multiple sclerosis), psychiatric disorders (e.g., psychosis, bipolar disorder, depression, neuropathic pain), conditions involving CNS-mediated chronic pain, spinal cord injuries, and other diseases or injuries.

The present application claims the benefit of priority to U.S. Provisional Application No. 60/916,273, filed May 4, 2007, the entire contents of this application being incorporated by reference.

The government owns rights in the present invention pursuant to grant number R01 CA78814 from the National Institutes of Health.

### BACKGROUND OF THE INVENTION

I. Field of the Invention

The present invention relates generally to the fields of biology and medicine. More particularly, it concerns compositions and methods for the treatment and prevention of diseases and injuries, including multiple sclerosis.

II. Description of Related Art

Multiple sclerosis (MS) continues to be a devastating neurological disease with fatal consequences in many patients. MS is believed to be an inflammatory autoimmune disease in which the patient's own T lymphocytes attack neurons, resulting in demyelination and subsequent neuronal failure. Multiple sclerosis may take several different forms, with new symptoms occurring either in discrete attacks or slowly accruing over time. Between attacks, symptoms may resolve completely, but permanent neurologic problems often persist, especially as the disease advances. MS currently does not have a cure, though several treatments are available that may slow the appearance of new symptoms.

MS causes gradual destruction of myelin (demyelination) and transection of neuron axons in patches throughout the brain and spinal cord, causing symptoms that vary widely depending upon which signals are interrupted. While there is no known definitive cure for multiple sclerosis, several types of treatments are used, depending on the MS type. The treatments include β-interferons, glatiramer acetate, mitoxantrone, natalizumab, and prednisone. Each of these therapies has significant side effects and limitations. For example, β-interferons reduce but don't eliminate flare-ups of multiple sclerosis. They have not been shown to reverse damage or significantly alter the long-term development of permanent disability. Also, some patients develop antibodies to β-interferons, which may make them less effective. The side effects of β-interferons may include flu-like symptoms. Glatiramer acetate is an alternative treatment to β-interferons for patients suffering from remitting MS; however, it was recently reported ineffective against the primary progressive types of the disease (Wolinsky et al., 2007), at least as a single agent treatment. Side effects of glatiramer acetate can include flushing and shortness of breath after injections, which are usually taken daily. Aggressive forms of relapsing remitting MS are often treated with mitoxantrone, a chemotherapy drug used for many cancers. The medication, while effective, is limited by cardiac toxicity. Finally, the use of the once promising treatment, natalizumab, has been sharply limited by the FDA, due to reports that it may lead to a rare, often fatal, brain disorder called progressive multifocal leukoencephalopathy.

Given the side effects and other limitation of the above methods of treating MS, and the lack of approved treatments for primary progressive multiple sclerosis, a need exists for new and more effective compounds and methods of treating and preventing this disease as well as other diseases, conditions and injuries affecting the central nervous system (CNS).

### SUMMARY OF THE INVENTION

The present invention overcomes limitations of the prior art by providing new compounds and methods for the treatment of various conditions, such as neurodegenerative diseases (e.g., multiple sclerosis), psychiatric disorders (e.g., psychosis, bipolar disorder, depression, neuropathic pain), conditions involving CNS-mediated chronic pain, spinal cord injuries, and other diseases or injuries affecting the CNS.

In one aspect, the method of treatment comprises administering to a subject a pharmaceutically effective amounts of a compound of formulas Ia, Ib, IIa or IIb, or pharmaceutically acceptable salts, esters, hydrates, solvates, tautomers, prodrugs, or optical isomers thereof.

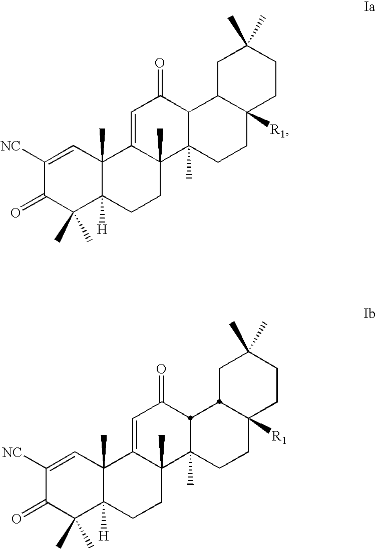

In formulas Ia and Ib, R₁ is a heteroatom-substituted or heteroatom-unsubstituted C₁-C₁₅-acyl.

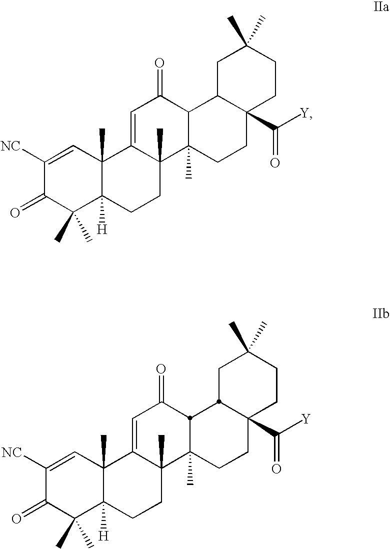

In formulas Ia and IIb, Y is —H, hydroxy, amino, halo, or a heteroatom-substituted or heteroatom-unsubstituted C₁-C₁₄-alkoxy, C₂-C₁₄-alkenyloxy, C₂-C₁₄-alkynyloxy, C₁-C₁₄-aryloxy, C₂-C₁₄-aralkoxy, C₁-C₁₄-alkylamino, C₂-C₁₄-alkenylamino, C₂-C₁₄-alkynylamino, C₁-C₁₄-arylamino, or C₂-C₁₄-aralkylamino. In some embodiments, Y is a heteroatom-substituted or heteroatom-unsubstituted C₂-C₄-alkylamino having at least one fluorine atom. In other embodiments, Y is a heteroatom-substituted or heteroatom-unsubstituted C₁-C₄-alkoxy. In some embodiments, the compound is a hydrate or a pharmaceutically acceptable salt of a compound according to formula Ib or IIb. In some embodiments, compounds used in the methods of the invention can be esters of the above formulas. An ester may, for example, result from a condensation reaction between a hydroxy group when present and the carboxylic acid group of biotin.

Non-limiting examples of compounds according to formulas Ia, Ib, IIa and IIb that may be used in accordance with the methods of this invention are shown below.

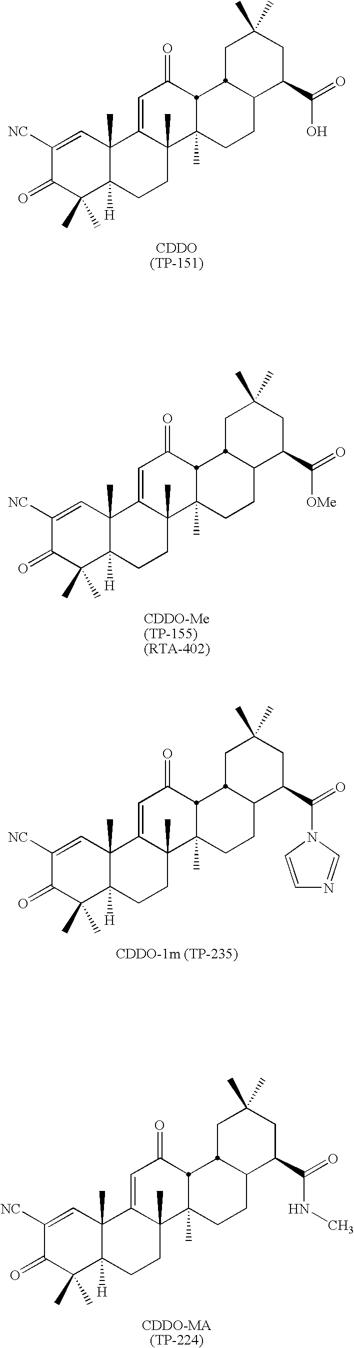

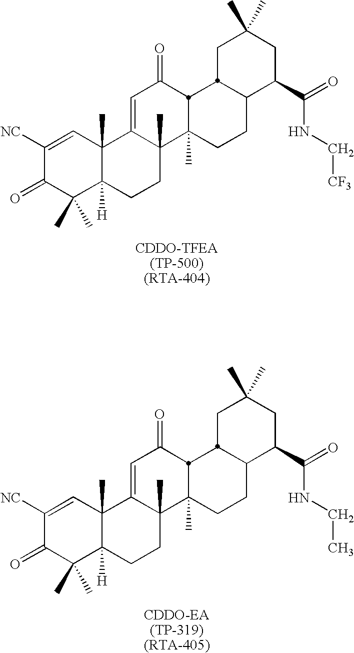

In some embodiments, the above compounds are administered as single enantiomers substantially free from optical isomers thereof. In other embodiments, the compounds are administered as a racemic mixture.

In some embodiments, the method may be used to treat MS, such as, primary progressive MS, relapsing-remitting MS, secondary progressive MS, or progressive relapsing MS. In some embodiments, the treatment may be used to suppress the demyelination of neurons in the subject's brain or spinal cord. In some embodiments, the treatment may be used to suppress one or more of the following conditions affecting the brains and/or spinal cords of a subject: inflammatory demyelination, transection of neuron axons, transection of neurites, and neuronal apoptosis.

In some embodiments, the treatment may be used to stimulate the remyelination of neuron axons in the brains or spinal cords of subjects. In some embodiments, the treatment may be used to restore lost function after an MS attack, prevent new MS attacks, and/or treat disability resulting from an MS attack.

In some embodiments, the subjects are primates, for example, humans. In other embodiments, the subjects can be cows, horses, dogs, cats, pigs, mice, rats, or guinea pigs.

In some embodiments, the method may be used to treat mental illness such as psychosis, major depression, bipolar disorder, or other neuropsychiatric disorders such as autism, attention deficit disorder, related disorders, and/or the symptoms thereof.

In some embodiments, the method may be used to treat neuropathic pain, fibromyalgia, other pain syndromes, related conditions (e.g., tinnitus), conditions that involve chronic activation of peripheral or CNS sensory pathways, and symptoms thereof.

In some embodiments, the method may be used to treat epilepsy and other seizure-related disorders.

In some embodiments, the method may be used to treat primary brain cancers such as glioblastoma and other gliomas, as well as metastatic brain cancer that develops secondary to non-CNS primary cancers such as breast cancer, lung cancer, prostate cancer, lymphoma, and melanoma.

In some embodiments, the method may be used to treat spinal cord injuries. In some embodiments, the treatment may be used to restore lost function related to the spinal cord injury. In some embodiments, the treatment may be used to prevent a disability related to the spinal cord injury.

A further aspect of the invention provides a method for treating multiple sclerosis (MS) in a subject comprising, administering to said subject a) a first amount of a first compound according to formula I or a pharmaceutically acceptable salt or hydrate thereof; and b) a second amount of a compound selected from the group consisting of interferon β-1 a, interferon β-1 b, glatiramer acetate, mitoxantrone, natalizumab, uric acid, and methylprednisolone; wherein the combined first and second amounts are effective to treat the MS.

In another aspect, the invention provides compounds of the formula III, or pharmaceutically acceptable salts, esters, hydrates, solvates, tautomers, prodrugs, or optical isomers thereof.

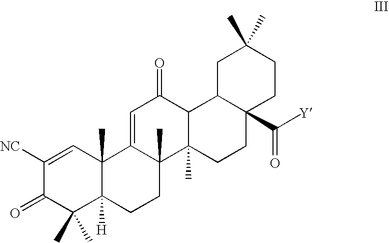

In formula III, Y′ is ethylamino or heteroatom-substituted C₁-C₅-alkylamino having at least one fluorine atom. In some variations, Y′ is a heteroatom-substituted or heteroatom-unsubstituted C₂-C₄-alkylamino having at least one fluorine atom. In some variations, the invention provides single enantiomers of these new synthetic triterpenoids or their salts or hydrates that are substantially free from other optical isomers thereof. The terms “compounds of the invention,” “compounds of the present invention,” “new CDDO derivatives” and “new synthetic triterpenoids” refers to compounds covered by formulas III and IV, as well as pharmaceutically acceptable salts, hydrates, solvates, tautomers, prodrugs, or optical isomers thereof.

In some embodiments, the invention provides compounds of the formula IV, or hydrates or pharmaceutically acceptable salts thereof.

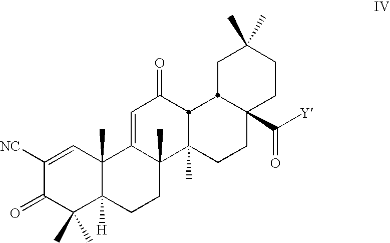

In formula IV, Y′ is ethylamino or heteroatom-substituted C₁-C₅-alkylamino having at least one fluorine atom. In some variations, Y′ is a heteroatom-substituted or heteroatom-unsubstituted C₂-C₄-alkylamino having at least one fluorine atom. In further embodiments, the invention provides pharmaceutically acceptable salts and hydrates of these new synthetic triterpenoids. In yet further embodiments, the invention provides single enantiomers of these new synthetic triterpenoids or their salts or hydrates that are substantially free from other optical isomers. In still further embodiments, racemic mixtures of these new synthetic triterpenoids as well as their salts and hydrates are provided.

Examples of new CDDO derivatives provided by the present invention include CDDO-TFEA and CDDO-EA, shown here.

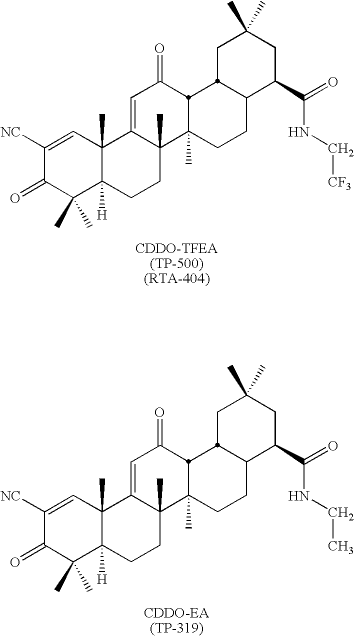

In some embodiments, the invention provides compounds selected from the groups consisting of:

(4aS,6aR,6bS,8aR,12aS,14aR,14bS)-11-cyano-N-ethyl-2,2,6a,6b,9,9,12a-heptamethyl-10,14-dioxo-1,2,3,4,4a,5,6,6a,6b,7,8,8a,9,10,12a,14,14a, 14b-octadecahydropicene-4a-carboxamide; and

(4aS,6aR,6bS,8aR,12aS,14aR,14bS)-11-cyano-2,2,6a,6b,9,9,12a-heptamethyl-10,14-dioxo-N-(2,2,2-trifluoroethyl)-1,2,3,4,4a,5,6,6a,6b,7,8,8a,9,10,12a,14,14a, 14b-octadecahydropicene-4a-carboxamide.

In some embodiments, compounds of the present invention are in the form of pharmaceutically acceptable salts. In other embodiments, compounds of the present invention are not be in the form of a pharmaceutically acceptable salts.

In some embodiments, the compounds of the present invention can be present as a mixture of stereoisomers. In other embodiments, the compounds of the present invention are present as single stereoisomers.

In some embodiments, compounds of the present invention may be inhibitors of IFN-γ-induced nitrous oxide (NO) production in macrophages, for example, having an IC₅₀ value of less than 0.2 μM.

In another aspect, the present invention provides pharmaceutical compositions comprising as an active ingredient a compound of the present invention and a pharmaceutically acceptable carrier. The composition may, for example, be adapted for administration by a route selected from the group consisting of orally, intraadiposally, intraarterially, intraarticularly, intracranially, intradermally, intralesionally, intramuscularly, intranasally, intraocularally, intrapericardially, intraperitoneally, intrapleurally, intraprostaticaly, intrarectally, intrathecally, intratracheally, intratumorally, intraumbilically, intravaginally, intravenously, intravesicularlly, intravitreally, liposomally, locally, mucosally, orally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, via localized perfusion, bathing target cells directly, or any combination thereof. In particular embodiments, the composition may be formulated for oral delivery. In particular embodiments, the composition is formulated as a hard or soft capsule, a tablet, a syrup, a suspension, a wafer, or an elixir. In certain embodiments, the soft capsule is a gelatin capsule. Certain compositions may comprise a protective coating, such as those compositions formulated for oral delivery. Certain compositions further comprise an agent that delays absorption, such as those compositions formulated for oral delivery. Certain compositions may further comprise an agent that enhances solubility or dispersibility, such as those compositions formulated for oral delivery. Certain compositions may comprise a compound of the present invention, wherein the compound is dispersed in a liposome, an oil and water emulsion or a water and oil emulsion.

Yet another general aspect of the present invention contemplates a therapeutic method comprising administering a pharmaceutically effective compound of the present invention to a subject. The subject may, for example, be a human. These or any other methods of the present invention may further comprise identifying a subject in need of treatment.

Another method of the present invention contemplates a method of treating cancer in a subject, comprising administering to the subject a pharmaceutically effective amount of a compound of the present invention. The cancer may be any type of cancer, such as a carcinoma, sarcoma, lymphoma, leukemia, melanoma, mesothelioma, multiple myeloma, or seminoma. Other types of cancers include cancer of the bladder, blood, bone, brain, breast, central nervous system, colon, endometrium, esophagus, genitourinary tract, head, larynx, liver, lung, neck, ovary, pancreas, prostate, spleen, small intestine, large intestine, stomach, or testicle. In these or any other methods, the subject may be a primate. This or any other method may further comprise identifying a subject in need of treatment. The subject may have a family or patient history of cancer. In certain embodiments, the subject has symptoms of cancer. The compounds of the invention may be administered via any method described herein, such as locally. In certain embodiments, the compound is administered by direct intratumoral injection or by injection into tumor vasculature. In certain embodiments, the compounds may be administered systemically. The compounds may be administered intravenously, intra-arterially, intramuscularly, intraperitoneally, subcutaneously or orally, in certain embodiments.

In certain embodiments regarding methods of treating cancer in a subject, comprising administering to the subject a pharmaceutically effective amount of a compound of the present invention, the pharmaceutically effective amount is 0.1-1000 mg/kg. In certain embodiments, the pharmaceutically effective amount is administered in a single dose per day. In certain embodiments, the pharmaceutically effective amount is administered in two or more doses per day. The compound may be administered by contacting a tumor cell during ex vivo purging, for example. The method of treatment may comprise any one or more of the following: a) inducing cytotoxicity in a tumor cell; b) killing a tumor cell; c) inducing apoptosis in a tumor cell; d) inducing differentiation in a tumor cell; or e) inhibiting growth in a tumor cell. The tumor cell may be any type of tumor cell, such as a leukemia cell. Other types of cells include, for example, a bladder cancer cell, a breast cancer cell, a lung cancer cell, a colon cancer cell, a prostate cancer cell, a liver cancer cell, a pancreatic cancer cell, a stomach cancer cell, a testicular cancer cell, a brain cancer cell, an ovarian cancer cell, a lymphatic cancer cell, a skin cancer cell, a brain cancer cell, a bone cancer cell, or a soft tissue cancer cell.

Combination treatment therapy is also contemplated by the present invention. For example, regarding methods of treating cancer in a subject, comprising administering to the subject a pharmaceutically effective amount of a compound of the present invention, the method may further comprise a treatment selected from the group consisting of administering a pharmaceutically effective amount of a second drug, radiotherapy, gene therapy, and surgery. Such methods may further comprise (1) contacting a tumor cell with the compound prior to contacting the tumor cell with the second drug, (2) contacting a tumor cell with the second drug prior to contacting the tumor cell with the compound, or (3) contacting a tumor cell with the compound and the second drug at the same time. The second drug may, in certain embodiments, be an antibiotic, anti-inflammatory, anti-neoplastic, anti-proliferative, anti-viral, immunomodulatory, or immunosuppressive. The second drug may be an alkylating agent, androgen receptor modulator, cytoskeletal disruptor, estrogen receptor modulator, histone-deacetylase inhibitor, HMG-CoA reductase inhibitor, prenyl-protein transferase inhibitor, retinoid receptor modulator, topoisomerase inhibitor, or tyrosine kinase inhibitor. In certain embodiments, the second drug is 5-azacitidine, 5-fluorouracil, 9-cis-retinoic acid, actinomycin D, alitretinoin, all-trans-retinoic acid, annamycin, axitinib, belinostat, bevacizumab, bexarotene, bosutinib, busulfan, capecitabine, carboplatin, carmustine, CD437, cediranib, cetuximab, chlorambucil, cisplatin, cyclophosphamide, cytarabine, dacarbazine, dasatinib, daunorubicin, decitabine, docetaxel, dolastatin-10, doxifluridine, doxorubicin, doxorubicin, epirubicin, erlotinib, etoposide, etoposide, gefitinib, gemcitabine, gemtuzamab ozogamicin, hexamethylmelamine, idarubicin, ifosfamide, imatinib, irinotecan, isotretinoin, ixabepilone, lapatinib, LBH589, lomustine, mechlorethamine, melphalan, mercaptopurine, methotrexate, mitomycin, mitoxantrone, MS-275, neratinib, nilotinib, nitrosourea, oxaliplatin, paclitaxel, plicamycin, procarbazine, semaxanib, semustine, sodium butyrate, sodium phenylacetate, streptozotocin, suberoylanilide hydroxamic acid, sunitinib, tamoxifen, teniposide, thiopeta, thioguanine, topotecan, TRAIL, trastuzumab, tretinoin, trichostatin A, valproic acid, valrubicin, vandetanib, vinblastine, vincristine, vindesine, or vinorelbine.

Methods of treating or preventing a disease with an inflammatory component in a subject, comprising administering to the subject a pharmaceutically effective amount of a compound of the present invention are also contemplated. The disease may be, for example, lupus or rheumatoid arthritis. The disease may be an inflammatory bowel disease, such as Crohn's disease or ulcerative colitis. The disease with an inflammatory component may be a cardiovascular disease. The disease with an inflammatory component may be diabetes, such as type 1 or type 2 diabetes. Compounds of the present invention may also be used to treat complications associated with diabetes. Such complications are well-known in the art and include, for example, obesity, hypertension, atherosclerosis, coronary heart disease, stroke, peripheral vascular disease, hypertension, nephropathy, neuropathy, myonecrosis, retinopathy and metabolic syndrome (syndrome X). The disease with an inflammatory component may be a skin disease, such as psoriasis, acne, or atopic dermatitis. Administration of a compound of the present invention in treatment methods of such skin diseases may be, for example, topical or oral.

The disease with an inflammatory component may be metabolic syndrome (syndrome X). A patient having this syndrome is characterized as having three or more symptoms selected from the following group of five symptoms: (1) abdominal obesity; (2) hypertriglyceridemia; (3) low high-density lipoprotein cholesterol (HDL); (4) high blood pressure; and (5) elevated fasting glucose, which may be in the range characteristic of Type 2 diabetes if the patient is also diabetic. Each of these symptoms is defined in the Third Report of the National Cholesterol Education Program Expert Panel on Detection, Evaluation and Treatment of High Blood Cholesterol in Adults (Adult Treatment Panel III, or ATP III), National Institutes of Health, 2001, NIH Publication No. 01-3670, incorporated herein by reference. Patients with metabolic syndrome, whether or not they have or develop overt diabetes mellitus, have an increased risk of developing the macrovascular and microvascular complications that are listed above that occur with type 2 diabetes, such as atherosclerosis and coronary heart disease.

Another general method of the present invention entails a method of treating or preventing a cardiovascular disease in a subject, comprising administering to the subject a pharmaceutically effective amount of a compound of the present invention. The cardiovascular disease may be, for example, atherosclerosis, cardiomyopathy, congenital heart disease, congestive heart failure, myocarditis, rheumatic heart disease, valve disease, coronary artery disease, endocarditis, or myocardial infarction. Combination therapy is also contemplated for such methods. For example, such methods may further comprise administering a pharmaceutically effective amount of a second drug. The second drug may be, for example, a cholesterol lowering drug, an anti-hyperlipidemic, a calcium channel blocker, an anti-hypertensive, or an HMG-CoA reductase inhibitor. Non-limiting examples of second drugs include amlodipine, aspirin, ezetimibe, felodipine, lacidipine, lercanidipine, nicardipine, nifedipine, nimodipine, nisoldipine or nitrendipine. Other non-limiting examples of second drugs include atenolol, bucindolol, carvedilol, clonidine, doxazosin, indoramin, labetalol, methyldopa, metoprolol, nadolol, oxprenolol, phenoxybenzamine, phentolamine, pindolol, prazosin, propranolol, terazosin, timolol or tolazoline. The second drug may be, for example, a statin, such as atorvastatin, cerivastatin, fluvastatin, lovastatin, mevastatin, pitavastatin, pravastatin, rosuvastatin or simvastatin.

Methods of treating or preventing a neurodegenerative disease in a subject, comprising administering to the subject a pharmaceutically effective amount of a compound of the present invention are also contemplated. The neurodegenerative disease may, for example, be selected from the group consisting of Parkinson's disease, Alzheimer's disease, multiple sclerosis (MS), Huntington's disease and amyotrophic lateral sclerosis. In particular embodiments, the neurodegenerative disease is Alzheimer's disease. In particular embodiments, the neurodegenerative disease is MS, such as primary progressive, relapsing-remitting secondary progressive or progressive relapsing MS. The subject may be, for example, a primate. The subject may be a human.

In particular embodiments of methods of treating or preventing a neurodegenerative disease in a subject, comprising administering to the subject a pharmaceutically effective amount of a compound of the present invention, the treatment suppresses the demyelination of neurons in the subject's brain or spinal cord. In certain embodiments, the treatment suppresses inflammatory demyelination. In certain embodiments, the treatment suppresses the transection of neuron axons in the subject's brain or spinal cord. In certain embodiments, the treatment suppresses the transection of neurites in the subject's brain or spinal cord. In certain embodiments, the treatment suppresses neuronal apoptosis in the subject's brain or spinal cord. In certain embodiments, the treatment stimulates the remyelination of neuron axons in the subject's brain or spinal cord. In certain embodiments, the treatment restores lost function after an MS attack. In certain embodiments, the treatment prevents a new MS attack. In certain embodiments, the treatment prevents a disability resulting from an MS attack.

One general aspect of the present invention contemplates a method of treating or preventing a disorder characterized by overexpression of iNOS genes in a subject, comprising administering to the subject a pharmaceutically effective amount of a compound of the present invention.

Another general aspect of the present invention contemplates a method of inhibiting IFN-γ-induced nitric oxide production in cells of a subject, comprising administering to said subject a pharmaceutically effective amount of a compound of the present invention.

Yet another general method of the present invention contemplates a method of treating or preventing a disorder characterized by overexpression of COX-2 genes in a subject, comprising administering to the subject a pharmaceutically effective amount of compound of the present invention.

Methods of treating renal/kidney disease (RKD) in a subject, comprising administering to the subject a pharmaceutically effective amount of a compound of the present invention are also contemplated. The RKD may result from, for example, a toxic insult. The toxic insult may result from, for example, an imaging agent or a drug. The drug may be a chemotherapeutic, for example. The RKD may result from ischemia/reperfusion injury, in certain embodiments. In certain embodiments, the RKD results from diabetes or hypertension. The RKD may result from an autoimmune disease. The RKD may be further defined as chronic RKD, or acute RKD.

In certain methods of treating renal/kidney disease (RKD) in a subject, comprising administering to the subject a pharmaceutically effective amount of a compound of the present invention, the subject has undergone or is undergoing dialysis. In certain embodiments, the subject has undergone or is a candidate to undergo kidney transplant. The subject may be a primate. The primate may be a human. The subject in this or any other method may be, for example, a cow, horse, dog, cat, pig, mouse, rat or guinea pig.

Also contemplated by the present invention is a method for improving glomerular filtration rate or creatinine clearance in a subject, comprising administering to the subject a pharmaceutically effective amount of a compound of the present invention.

Kits are also contemplated by the present invention, such as a kit comprising: a compound of the present invention; and instructions which comprise one or more forms of information selected from the group consisting of indicating a disease state for which the compound is to be administered, storage information for the compound, dosing information and instructions regarding how to administer the compound. The kit may comprise a compound of the present invention in a multiple dose form.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula. Any embodiment discussed herein with respect to one aspect of the invention applies to other aspects of the invention as well, unless specifically noted.

Other objects, features and advantages of the present invention will become apparent from the following detailed description and any accompanying drawings. It should be understood, however, that the detailed description and any specific examples or drawings provided, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1—CDDO-Me (TP-155) is Detectable in the Brains of Mice Fed Very Low Levels of Compound for One Week. Three male mice were in each group. The concentration in picograms (pg) of TP-155 per milligrams (mg) of mouse brain is shown as a function of the amount of Tp-155 in the diet, normalized to the weight of the individual mouse.

FIG. 2—Significant Concentrations of CDDO Methyl Amide (TP-224) in Brains of Mice After Feeding 800 mg/kg Diet. The nanomolar concentration of TP-244 in the brains of mice is shown as a function of the number of days the mice were fed a 800 mg/kg diet of TP-224.

FIG. 3—Feeding CDDO-Ethyl Amide (TP-319) for Two Days Results in Significantly Higher Brain Levels Than CDDO Methyl Amide (TP-224). Four CD-1 mice per group were fed triterpenoids (800 mg/kg diet) for 48 hrs, and triterpenoid levels in brain were analyzed by LC/MS.

FIG. 4—Brain Levels of CDDO-Ethyl Amide (TP-319) Are Dose Responsive and Higher Than For CDDO Methyl Amide (TP-224). Male CD-1 mice were fed triterpenoids (200, 400 or 800 mg/kg diet) for 3.5 days, and triterpenoid levels in the brains of the mice were analyzed by LC/MS. The number of mice in each experiment is indicated by “n”.

FIG. 5—CDDO-TFEA (TP-500) Is Detected at Higher Levels in Mouse Brain than CDDO-EA (TP-319). CD-1 mice were fed either 200 or 400 mg/kg diet of either TP-319 or TP-500 for 3.5 days, and TP levels in the brains of the mice were analyzed by LC/MS.

FIG. 6—Brain Levels of CDDO-TFEA (TP-500) Remain Significantly Higher Than CDDO-EA (TP-319). Four CD-1 mice per group were fed TPs (400 mg/kg diet) for 10 weeks (CDDO-EA) or 6 weeks (CDDO-TFEA), and TP levels in the brains of the mice were analyzed by LC/MS.

FIG. 7—Brain Levels of Triterpenoids in Gavaged CD-1 Mice. Male CD-1 mice, which each group containing “n” mice, were gavaged with TPs (2 μmol/mouse) daily for 3 consecutive days. Six hours after the final dose, TP levels in brain were analyzed by LC/MS.

FIG. 8—CDDO-EA (TP-319) in CD-1 Mouse Tissues. Four male CD-1 mice per group were gavaged once daily for 3 consecutive days with 1 μmol TP-319 (CDDO-EA). Six hours after the final gavage, the mice were sacrificed and TP levels were analyzed by LC/MS.

FIG. 9—CDDO-TFEA (TP-500) in CD-1 Mouse Tissues. Four male CD-1 mice per group were gavaged once daily for 3 consecutive days with 1 μmol TP-500 (CDDO-EA). Six hours after the final gavage, the mice were sacrificed and TP levels were analyzed by LC/MS.

FIGS. 10 and 11—CDDO-TFEA (RTA 404) and CDDO-Me (RTA-402)

Induce Full Recovery of Mice in Rapidly Progressive EAE Model. All animals (n=2/group) of varying clinical scores (CS) were immunized with myelin oligodendrocyte glycoprotein (MOG). The dose of MOG Peptide was 200 μg (divided into two injections, 100 μl each). The animals were then treated intraperitoneally (IP) with 100 nmol (˜2.8 mg/kg) of RTA-402 or RTA-404 in 7.5% PBST (Phosphate Buffered Saline Tween-20) on a Q2D×4 (4 doses, one every other day) schedule. A CS score of 0 indicates no symptoms, and score of 6 indicates quadriplegia.

FIGS. 12, 13, 14, 15, 16 and 17—Untreated Animals do not Survive and Treated Animals Recover. “CDDO-CF₃” refers to CDDO-TFEA. All animals (n=2/group) of varying clinical scores (CS) were immunized with myelin oligodendrocyte glycoprotein (MOG). The dose of MOG Peptide was 200 μg (divided into two injections, 100 μl each). The animals were then treated intraperitoneally (IP) with 100 nmol (˜2.8 mg/kg) of RTA-402 or RTA-404 in 7.5% PBST (Phosphate Buffered Saline Tween-20) on a Q2D×4 (4 doses, one every other day) schedule. A CS score of 0 indicates no symptoms, and score of 6 indicates quadriplegia.

FIGS. 18-22—Synthetic Triterpenoids Induce Remission in MOG-Induced EAE Model of MS. All animals (n=2/group) of varying clinical scores (CS) were immunized with myelin oligodendrocyte glycoprotein (MOG). The dose of MOG Peptide was 200 μg (divided into two injections, 100 μl each). The animals were then treated intraperitoneally (IP) with 100 nmol (˜2.8 mg/kg) of synthetic triterpenoids (or control), RTA-404 in FIG. 18, control in FIG. 19, RTA-402 in FIG. 20, RTA-405 in FIG. 21, and RTA-404 in FIG. 22 in 7.5% PBST (Phosphate Buffered Saline Tween-20) on a Q2D×4 (4 doses, one every other day) schedule. A CS score of 0 indicates no symptoms, and score of 6 indicates quadriplegia. RTA 404, RTA 402, and RTA 405 induce complete recovery of symptoms after initial relapse. Severity of symptoms post-initial treatment, recovery, and then relapse is generally less severe and not lethal. Time to second relapse is much longer than first, and fewer RTA 404 and RTA 405 treated animals relapse. All untreated animals (FIG. 19) succumbed to paralysis during the same time frame.

FIGS. 23 & 24—Prophylactic Treatment of CDDO-Me (RTA 402) or CDDO-TFEA (RTA-404) Delays Induction of Symptoms in Model of Multiple Sclerosis. Development of clinical scores can be moderately delayed with pre and/or post-treatment of RTA 404 (FIG. 24) and modestly delayed with similar schedules of RTA 402 (FIG. 23).

FIG. 25—Histologic Evidence of Resolution of Inflammatory Lesions in the Brain after CDDO-TFEA Treatment. The three panels show H&E stains of tissue harvested from the brain stems of mice. The left panel shows the H&E stain from the control group, a mouse that was neither immunized with MOG nor treated with TP. The middle panel shows extensive inflammation (here in the brainstem, but present in spinal cord and brain cortex as well) of a mouse that had been immunized with 200 μg of MOG (divided into two injections, 100 μl each) and had expired approximately 15 to 18 days later. The H&E stain reveals significant perivascular infiltrates (indicated by arrows) and infiltrates along the surface of the brain (subdural). These are gone in a treated animal (vessels encircled are free of surrounding infiltrates as is the surface of the brainstem), as shown in the right panel. The tissue of the brain stem of the treated animal was harvested after the mouse had recovered to a CS of 0 after having been first immunized with 200 μg of MOG (divided into two injections, 100 μl each), second allowed to degenerate to a CS of 6, third treated intraperitoneally (IP) with 100 nmol (˜2.8 mg/kg) of CDDO-TFEA in 7.5% PBST (Phosphate Buffered Saline Tween-20) on a Q2D×4 (4 doses, one every other day) schedule, and fourth allowed to recover to a CS of 0. After treatment, no significant infiltrate observed in brains.

FIG. 26—Histologic Evidence of Resolution of Inflammatory Lesions in the Spinal Cord after CDDO-TFEA Treatment. EAE-induced animals developed significant peri-vascular and surface infiltration at score of 5. Inflammatory infiltrate in CDDO-TFEA-treated animals were similar to controls after treatment. After treatment minimal peri-vascular and no significant surface infiltrate observed in cord, showing that CDDO-TFEA reduces inflammation in brain and spinal cord of symptomatic animals.

FIG. 27—Histologic Evidence of Recovery of Myelin Content in Spinal Cord Promoted by CDDO-TFEA Treatment. Panels show Luxol fast blue staining of spinal cord. CDDO-TFEA (“Treated”) Promotes Recovery of Myelin Content. Staining of myelin demonstrates depletion in EAE-induced animals (“Untreated”) throughout spinal cord. CDDO-TFEA-treated animals' myelin levels approach control levels, after having been at score of 5 pre-treatment and returned to 0 post-treatment.

FIGS. 28 & 29—CDDO-TFEA Eliminates Brain iNOS and Significantly Reduces Spinal Cord iNOS Expression. Inoculation with MOG induces strong expression of iNOS in the brain (FIG. 28) and spinal cord (FIG. 29). Untreated refers to EAE model with no treatment. Treated refers to EAE model with treatment.

FIGS. 30A-G—CDDO-TFEA Suppresses Th1 and Th2 Cytokines Induced in MOG EAE Model. Inoculation with MOG (“untreated”) induces multiple Th1 and Th2 pro-inflammatory cytokines in circulation. CDDO-TFEA (“treated”) suppresses cytokines levels back to or near baseline (“control”).

FIG. 31—RTA 404 and RTA 405 Suppress MOG-Induced T cell Proliferation in Mutant and Wild-type Animals. All mice injected with CFA and MOG in both flanks. Mice sacrificed 14 days after injection and total cells of draining lymph nodes were cultured for 96 hours with or without MOG. In last 24 hours, cells were treated with vehicle or 10 nM of RTA 404 or RTA 405. ³H-thymidine incorporation was measured for last 24 hours. TGFb and SMAD3 heterozygous animals were used, which accelerates development of EAE pathology and symptoms. RTA 404 and RTA 405 suppressed T cell proliferation in cultures from both wild-type and mutant animals.

FIGS. 32A-E—CDDO-TFEA (RTA 404) Effective in Relapsing-Remitting Model of MS. Female SJL/J mice (n=22) were immunized with PLP (139-151). All mice were then divided into groups of the same clinical score (CS) on day 12. FIG. 32A shows group 1 starting with a CS of 0 (n=6); FIG. 32B shows Group 2 starting with a CS of 1 (n=9); FIG. 32C shows Group 3 starting with a CS of 2 (n=5); FIG. 32D shows Group 4 starting with a CS of 3 (n=1), and FIG. 32E shows Group 5 starting with a CS of 4 (n=1). In each group, mice were treated with 0.29 mg/kg RTA 404 IP or vehicle IP every 2 days starting on Day 13.

FIG. 33—Immunofluorescent Panels of CDDO-TFEA (RTA 404) Induced Myelin Repair in Model of Direct Myelin Injury. Experimental design: Day 1: 5 μL of lysophosphatidylcholine (LPC), a component of oxidized low-density lipoprotein (LDL) was injected locally into the spinal cord of a Wistar rat in order to induce myelin disruption/destruction. (PBS was used as a control.) On Day 3 (48 hours later), a single dose, 1.0 mmole of RTA 404 in 100 μL PBS or PBS (control) was administered by IP injection. On Day 10, the animals were perfused with PBS to remove all blood prior to tissue fixation. The third column of plates shows an overlay of the first two columns [please confirm]. Oligodendrocyte; M: Myelin; A: Astrocyte.

FIG. 34—Electron Microscope Images of Myelin Repair Induction by CDDO-TFEA. Images correspond to same experimental design detailed in description to FIG. 33, above.

### DESCRIPTION OF ILLUSTRATIVE EMBODIMENTS

### I. The Present Invention

The present invention concerns new compounds and methods for the treatment and prevention of diseases, conditions and injuries affecting the CNS, including multiple sclerosis (MS).

### II. Definitions

As used herein, the term “amino” means —NH₂; the term “nitro” means —NO₂; the term “halo” designates —F, —Cl, —Br or —I; the term “mercapto” means —SH; the term “cyano” means —CN; the term “silyl” means —SiH₃, and the term “hydroxy” means —OH.

The term “heteroatom-substituted,” when used to modify a class of organic radicals (e.g., alkyl, aryl, acyl, etc.), means that one, or more than one, hydrogen atom of that radical has been replaced by a heteroatom, or a heteroatom containing group. Examples of heteroatoms and heteroatom containing groups include: halo, hydroxy, cyano, alkoxy, ═O, ═S, —NO₂, —N(CH₃)₂, amino, or —SH. Specific heteroatom-substituted organic radicals are defined more fully below.

The term “heteroatom-unsubstituted,” when used to modify a class of organic radicals (e.g., alkyl, aryl, acyl, etc.) means that none of the hydrogen atoms of that radical have been replaced with a heteroatom or a heteroatom containing group. Substitution of a hydrogen atom with a carbon atom, or a group consisting of only carbon and hydrogen atoms, is not sufficient to make a group heteroatom-substituted. For example, the group —C₆H₄C≡CH is an example of a heteroatom-unsubstituted aryl group, while —C₆H₄F is an example of a heteroatom-substituted aryl group. Specific heteroatom-unsubstituted organic radicals are defined more fully below.

The term “heteroatom-unsubstituted Cₙ-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C₁-C₁₀-alkyl has 1 to 10 carbon atoms. The term “alkyl” includes straight-chain alkyl groups, branched-chain alkyl groups, cycloalkyl (alicyclic) groups, alkyl heteroatom-substituted cycloalkyl groups, and cycloalkyl heteroatom-substituted alkyl groups. The groups, —CH₃, —CH₂CH₃, —CH₂CH₂CH₃, —CH(CH₃)₂, —CH(CH₂)₂ (cyclopropyl), —CH₂CH₂CH₂CH₃, —CH(CH₃)CH₂CH₃, —CH₂CH(CH₃)₂, —C(CH₃)₃, —CH₂C(CH₃)₃, cyclobutyl, cyclopentyl, and cyclohexyl, are all examples of heteroatom-unsubstituted alkyl groups.

The term “heteroatom-substituted Cₙ-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkyl has 1 to 10 carbon atoms. The following groups are all examples of heteroatom-substituted alkyl groups: trifluoromethyl, —CH₂F, —CH₂Cl, —CH₂Br, —CH₂OH, —CH₂OCH₃, —CH₂OCH₂CH₃, —CH₂OCH₂CH₂CH₃, —CH₂OCH(CH₃)₂, —CH₂OCH(CH₂)₂, —CH₂OCH₂CF₃, —CH₂OCOCH₃, —CH₂NH₂, —CH₂NHCH₃, —CH₂N(CH₃)₂, —CH₂NHCH₂CH₃, —CH₂N(CH₃)CH₂CH₃, —CH₂NHCH₂CH₂CH₃, —CH₂NHCH(CH₃)₂, —CH₂NHCH(CH₂)₂, —CH₂N(CH₂CH₃)₂, —CH₂CH₂F, —CH₂CH₂Cl, —CH₂CH₂Br, —CH₂CH₂I, —CH₂CH₂OH, CH₂CH₂OCOCH₃, —CH₂CH₂NH₂, —CH₂CH₂N(CH₃)₂, —CH₂CH₂NHCH₂CH₃, —CH₂CH₂N(CH₃)CH₂CH₃, —CH₂CH₂NHCH₂CH₂CH₃, —CH₂CH₂NHCH(CH₃)₂, —CH₂CH₂NHCH(CH₂)₂, —CH₂CH₂N(CH₂CH₃)₂, —CH₂CH₂NHCO₂C(CH₃)₃, and —CH₂Si(CH₃)₃.

The term “heteroatom-unsubstituted Cₙ-alkenyl” refers to a radical, having a linear or branched, cyclic or acyclic structure, further having at least one nonaromatic carbon-carbon double bond, but no carbon-carbon triple bonds, a total of n carbon atoms, three or more hydrogen atoms, and no heteroatoms. For example, a heteroatom-unsubstituted C₂-C₁₀-alkenyl has 2 to 10 carbon atoms. Heteroatom-unsubstituted alkenyl groups include: —CH═CH₂, —CH═CHCH₃, —CH═CHCH₂CH₃, —CH═CHCH₂CH₂CH₃, —CH═CHCH(CH₃)₂, —CH═CHCH(CH₂)₂, —CH₂CH═CH₂, —CH₂CH═CHCH₃, —CH₂CH═CHCH₂CH₃, —CH₂CH═CHCH₂CH₂CH₃, —CH₂CH═CHCH(CH₃)₂, —CH₂CH═CHCH(CH₂)₂, and —CH═CH—C₆H₅.

The term “heteroatom-substituted Cₙ-alkenyl” refers to a radical, having a single nonaromatic carbon atom as the point of attachment and at least one nonaromatic carbon-carbon double bond, but no carbon-carbon triple bonds, further having a linear or branched, cyclic or acyclic structure, further having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom-substituted C₂-C₁₀-alkenyl has 2 to 10 carbon atoms. The groups, —CH═CHF, —CH═CHCl and —CH═CHBr, are examples of heteroatom-substituted alkenyl groups.

The term “heteroatom-unsubstituted Cₙ-alkynyl” refers to a radical, having a linear or branched, cyclic or acyclic structure, further having at least one carbon-carbon triple bond, a total of n carbon atoms, at least one hydrogen atom, and no heteroatoms. For example, a heteroatom-unsubstituted C₂-C₁₀-alkynyl has 2 to 10 carbon atoms. The groups, —C≡CH, —C≡CCH₃, and —C≡CC₆H₅ are examples of heteroatom-unsubstituted alkynyl groups.

The term “heteroatom-substituted Cₙ-alkynyl” refers to a radical, having a single nonaromatic carbon atom as the point of attachment and at least one carbon-carbon triple bond, further having a linear or branched, cyclic or acyclic structure, and having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom-substituted C₂-C₁₀-alkynyl has 2 to 10 carbon atoms. The group, —C≡CSi(CH₃)₃, is an example of a heteroatom-substituted alkynyl group.

The term “heteroatom-unsubstituted Cₙ-aryl” refers to a radical, having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom-unsubstituted C₆-C₁₀-aryl has 6 to 10 carbon atoms. Examples of heteroatom-unsubstituted aryl groups include phenyl, methylphenyl, (dimethyl)phenyl, —C₆H₄—CH₂CH₃, —C₆H₄CH₂CH₂CH₃, —C₆H₄CH(CH₃)₂, —C₆H₄CH(CH₂)₂, —C₆H₃(CH₃)CH₂CH₃, —C₆H₄CH═CH₂, —C₆H₄CH═CHCH₃, —C₆H₄C≡CH, —C₆H₄C≡CCH₃, naphthyl, quinolyl, indolyl, and the radical derived from biphenyl. The term “heteroatom-unsubstituted aryl” includes carbocyclic aryl groups, biaryl groups, and radicals derived from polycyclic fused hydrocarbons (PAHs).

The term “heteroatom-substituted Cₙ-aryl” refers to a radical, refers to a radical, having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C₁-C₁₀-heteroaryl has 1 to 10 carbon atoms. The term “heteroatom-substituted aryl” includes heteroaryl and heterocyclic aryl groups. It also includes those groups derived from the compounds: pyrrole, furan, thiophene, imidazole, oxazole, isoxazole, thiazole, isothiazole, triazole, pyrazole, pyridine, pyrazine, pyridazine, pyrimidine, and the like. Further examples of heteroatom-substituted aryl groups include the groups: —C₆H₄F, —C₆H₄Cl, —C₆H₄Br, —C₆H₄, —C₆H₄OH, —C₆H₄OCH₃, —C₆H₄OCH₂CH₃, —C₆H₄OCOCH₃, —C₆H₄C₆H₅, —C₆H₄NH₂, —C₆H₄NHCH₃, —C₆H₄NHCH₂CH₃, —C₆H₄CH₂Cl, —C₆H₄CH₂Br, —C₆H₄CH₂OH, —C₆H₄CH₂OCOCH₃, —C₆H₄CH₂NH₂, —C₆H₄N(CH₃)₂, —C₆H₄CH₂CH₂Cl, —C₆H₄CH₂CH₂OH, —C₆H₄CH₂CH₂OCOCH₃, —C₆H₄CH₂CH₂NH₂, —C₆H₄CH₂CH═CH₂, —C₆H₄CF₃, —C₆H₄CN, —C₆H₄C≡CSi(CH₃)₃, —C₆H₄COH, —C₆H₄COCH₃, —C₆H₄COCH₂CH₃, —C₆H₄COCH₂CF₃, —C₆H₄COC₆H₅, —C₆H₄CO₂H, —C₆H₄CO₂CH₃, —C₆H₄CONH₂, —C₆H₄CONHCH₃, —C₆H₄CON(CH₃)₂, furanyl, thienyl, pyridyl, pyrrolyl, pyrimidyl, pyrazinyl, and imidazoyl.

The term “heteroatom-unsubstituted Cₙ-aralkyl” refers to a radical, having a single saturated carbon atom as the point of attachment, further having a total of n carbon atoms, wherein at least 6 of the carbon atoms form an aromatic ring structure containing only carbon atoms, 7 or more hydrogen atoms, and no heteroatoms. For example, a heteroatom-unsubstituted C₇-C₁₀-aralkyl has 7 to 10 carbon atoms. An “aralkyl” includes an alkyl heteroatom-substituted with an aryl group. Examples of heteroatom-unsubstituted aralkyls include phenylmethyl (benzyl) and phenylethyl.

The term “heteroatom-substituted Cₙ-aralkyl” refers to a radical, having a single saturated carbon atom as the point of attachment, further having a total of n carbon atoms, 0, 1, or more than one hydrogen atom, and at least one heteroatom, wherein at least one of the carbon atoms is incorporated an aromatic ring structures, further wherein each heteroatom is independently selected from the group consisting of N, O, F, Cl, Br, I, Si, P, and S. For example, a heteroatom-substituted C₂-C₁₀-heteroaralkyl has 2 to 10 carbon atoms.

The term “heteroatom-unsubstituted Cₙ-acyl” refers to a radical, having a single carbon atom of a carbonyl group as the point of attachment, further having a linear or branched, cyclic or acyclic structure, further having a total of n carbon atoms, 1 or more hydrogen atoms, a total of one oxygen atom, and no additional heteroatoms. For example, a heteroatom-unsubstituted C₁-C₁₀-acyl has 1 to 10 carbon atoms. The groups, —COH, —COCH₃, —COCH₂CH₃, —COCH₂CH₂CH₃, —COCH(CH₃)₂, —COCH(CH₂)₂, —COC₆H₅, —COC₆H₄CH₃, —COC₆H₄CH₂CH₃, —COC₆H₄CH₂CH₂CH₃, —COC₆H₄CH(CH₃)₂, —COC₆H₄CH(CH₂)₂, and —COC₆H₃(CH₃)₂, are examples of heteroatom-unsubstituted acyl groups.

The term “heteroatom-substituted Cₙ-acyl” refers to a radical, having a single carbon atom as the point of attachment, the carbon atom being part of a carbonyl group, further having a linear or branched, cyclic or acyclic structure, further having a total of n carbon atoms, 0, 1, or more than one hydrogen atom, at least one additional heteroatom in addition to the oxygen of the carbonyl group, wherein each additional heteroatom is independently selected from the group consisting of N, O, F, Cl, Br, I, Si, P, and S. For example, a heteroatom-substituted C₁-C₁₀-acyl has 1 to 10 carbon atoms. The term heteroatom-substituted acyl includes carbamoyl, thiocarboxylate, and thiocarboxylic acid groups. The groups, —COCH₂CF₃, —CO₂H, —CO₂CH₃, —CO₂CH₂CH₃, —CO₂CH₂CH₂CH₃, —CO₂CH(CH₃)₂, —CO₂CH(CH₂)₂, —CONH₂, —CONHCH₃, —CONHCH₂CH₃, —CONHCH₂CH₂CH₃, —CONHCH(CH₃)₂, —CONHCH(CH₂)₂, —CON(CH₃)₂, —CON(CH₂CH₃)CH₃, —CON(CH₂CH₃)₂ and —CONHCH₂CF₃, are examples heteroatom-substituted acyl groups.

The term “heteroatom-unsubstituted Cₙ-alkoxy” refers to a group, having the structure —OR, in which R is a heteroatom-unsubstituted Cₙ-alkyl, as that term is defined above. Heteroatom-unsubstituted alkoxy groups include: —OCH₃, —OCH₂CH₃, —OCH₂CH₂CH₃, —OCH(CH₃)₂, and —OCH(CH₂)₂.

The term “heteroatom-substituted Cₙ-alkoxy” refers to a group, having the structure —OR, in which R is a heteroatom-substituted Cₙ-alkyl, as that term is defined above. For example, —OCH₂CF₃ is a heteroatom-substituted alkoxy group.

The term “heteroatom-unsubstituted Cₙ-alkenyloxy” refers to a group, having the structure —OR, in which R is a heteroatom-unsubstituted Cₙ-alkenyl, as that term is defined above.

The term “heteroatom-substituted Cₙ-alkenyloxy” refers to a group, having the structure —OR, in which R is a heteroatom-substituted Cₙ-alkenyl, as that term is defined above.

The term “heteroatom-unsubstituted Cₙ-alkynyloxy” refers to a group, having the structure —OR, in which R is a heteroatom-unsubstituted Cₙ-alkynyl, as that term is defined above.

The term “heteroatom-substituted Cₙ-alkynyloxy” refers to a group, having the structure —OR, in which R is a heteroatom-substituted Cₙ-alkynyl, as that term is defined above.

The term “heteroatom-unsubstituted Cₙ-aryloxy” refers to a group, having the structure —OAr, in which Ar is a heteroatom-unsubstituted Cₙ-aryl, as that term is defined above. An example of a heteroatom-unsubstituted aryloxy group is —OC₆H₅.

The term “heteroatom-substituted Cₙ-aryloxy” refers to a group, having the structure —OAr, in which Ar is a heteroatom-substituted Cₙ-aryl, as that term is defined above.

The term “heteroatom-unsubstituted Cₙ-aralkyloxy” refers to a group, having the structure —OAr, in which Ar is a heteroatom-unsubstituted Cₙ-aralkyl, as that term is defined above.

The term “heteroatom-substituted Cₙ-aralkyloxy” refers to a group, having the structure —OAr, in which Ar is a heteroatom-substituted Cₙ-aralkyl, as that term is defined above.

The term “heteroatom-unsubstituted Cₙ-acyloxy” refers to a group, having the structure —OAc, in which Ac is a heteroatom-unsubstituted Cₙ-acyl, as that term is defined above. A heteroatom-unsubstituted acyloxy group includes alkylcarbonyloxy and arylcarbonyloxy groups. For example, —OCOCH₃ is an example of a heteroatom-unsubstituted acyloxy group.

The term “heteroatom-substituted Cₙ-acyloxy” refers to a group, having the structure —OAc, in which Ac is a heteroatom-substituted Cₙ-acyl, as that term is defined above. A heteroatom-substituted acyloxy group includes alkoxycarbonyloxy, aryloxycarbonyloxy, carboxylate, alkylcarbonyl, alkoxycarbonyl, aminocarbonyl, and alkylthiocarbonyl groups.

The term “heteroatom-unsubstituted Cₙ-alkylamino” refers to a radical, having a single nitrogen atom as the point of attachment, further having one or two saturated carbon atoms attached to the nitrogen atom, further having a linear or branched, cyclic or acyclic structure, containing a total of n carbon atoms, all of which are nonaromatic, 4 or more hydrogen atoms, a total of 1 nitrogen atom, and no additional heteroatoms. For example, a heteroatom-unsubstituted C₁-C₁₀-alkylamino has 1 to 10 carbon atoms. The term “heteroatom-unsubstituted Cₙ-alkylamino” includes groups, having the structure —NHR, in which R is a heteroatom-unsubstituted Cₙ-alkyl, as that term is defined above. A heteroatom-unsubstituted alkylamino group would include —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —N(CH₃)₂, —N(CH₃)CH₂CH₃, —N(CH₂CH₃)₂, N-pyrrolidinyl, and N-piperidinyl.

The term “heteroatom-substituted Cₙ-alkylamino” refers to a radical, having a single nitrogen atom as the point of attachment, further having one or two saturated carbon atoms attached to the nitrogen atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₁-C₁₀-alkylamino has 1 to 10 carbon atoms. The term “heteroatom-substituted Cₙ-alkylamino” includes groups, having the structure —NHR, in which R is a heteroatom-substituted Cₙ-alkyl, as that term is defined above.

The term “heteroatom-unsubstituted Cₙ-alkenylamino” refers to a radical, having a single nitrogen atom as the point of attachment, further having one or two carbon atoms attached to the nitrogen atom, further having a linear or branched, cyclic or acyclic structure, containing at least one nonaromatic carbon-carbon double bond, a total of n carbon atoms, 4 or more hydrogen atoms, a total of one nitrogen atom, and no additional heteroatoms. For example, a heteroatom-unsubstituted C₂-C₁₀-alkenylamino has 2 to 10 carbon atoms. The term “heteroatom-unsubstituted Cₙ-alkenylamino” includes groups, having the structure —NHR, in which R is a heteroatom-unsubstituted Cₙ-alkenyl, as that term is defined above. Examples of heteroatom-unsubstituted Cₙ-alkenylamino groups also include dialkenylamino and alkyl(alkenyl)amino groups.

The term “heteroatom-substituted Cₙ-alkenylamino” refers to a radical, having a single nitrogen atom as the point of attachment and at least one nonaromatic carbon-carbon double bond, but no carbon-carbon triple bonds, further having one or two carbon atoms attached to the nitrogen atom, further having a linear or branched, cyclic or acyclic structure, further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkenylamino has 2 to 10 carbon atoms. The term “heteroatom-substituted Cₙ-alkenylamino” includes groups, having the structure —NHR, in which R is a heteroatom-substituted Cₙ-alkenyl, as that term is defined above.

The term “heteroatom-unsubstituted Cₙ-alkynylamino” refers to a radical, having a single nitrogen atom as the point of attachment, further having one or two carbon atoms attached to the nitrogen atom, further having a linear or branched, cyclic or acyclic structure, containing at least one carbon-carbon triple bond, a total of n carbon atoms, at least one hydrogen atoms, a total of one nitrogen atom, and no additional heteroatoms. For example, a heteroatom-unsubstituted C₂-C₁₀-alkynylamino has 2 to 10 carbon atoms. The term “heteroatom-unsubstituted Cₙ-alkynylamino” includes groups, having the structure —NHR, in which R is a heteroatom-unsubstituted Cₙ-alkynyl, as that term is defined above. An alkynylamino group includes dialkynylamino and alkyl(alkynyl)amino groups.

The term “heteroatom-substituted Cₙ-alkynylamino” refers to a radical, having a single nitrogen atom as the point of attachment, further having one or two carbon atoms attached to the nitrogen atom, further having at least one nonaromatic carbon-carbon triple bond, further having a linear or branched, cyclic or acyclic structure, and further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkynylamino has 2 to 10 carbon atoms. The term “heteroatom-substituted Cₙ-alkynylamino” includes groups, having the structure —NHR, in which R is a heteroatom-substituted Cₙ-alkynyl, as that term is defined above.

The term “heteroatom-unsubstituted Cₙ-arylamino” refers to a radical, having a single nitrogen atom as the point of attachment, further having at least one aromatic ring structure attached to the nitrogen atom, wherein the aromatic ring structure contains only carbon atoms, further having a total of n carbon atoms, 6 or more hydrogen atoms, a total of one nitrogen atom, and no additional heteroatoms. For example, a heteroatom-unsubstituted C₆-C₁₀-arylamino has 6 to 10 carbon atoms. The term “heteroatom-unsubstituted Cₙ-arylamino” includes groups, having the structure —NHR, in which R is a heteroatom-unsubstituted Cₙ-aryl, as that term is defined above. A heteroatom-unsubstituted arylamino group includes diarylamino and alkyl(aryl)amino groups.

The term “heteroatom-substituted Cₙ-arylamino” refers to a radical, having a single nitrogen atom as the point of attachment, further having a total of n carbon atoms, at least one hydrogen atom, at least one additional heteroatoms, that is, in addition to the nitrogen atom at the point of attachment, wherein at least one of the carbon atoms is incorporated into one or more aromatic ring structures, further wherein each additional heteroatom is independently selected from the group consisting of N, O, F, Cl, Br, I, Si, P, and S. For example, a heteroatom-substituted C₆-C₁₀-arylamino has 6 to 10 carbon atoms. The term “heteroatom-substituted Cₙ-arylamino” includes groups, having the structure —NHR, in which R is a heteroatom-substituted Cₙ-aryl, as that term is defined above. A heteroatom-substituted arylamino group includes heteroarylamino groups.

The term “heteroatom-unsubstituted Cₙ-aralkylamino” refers to a radical, having a single nitrogen atom as the point of attachment, further having one or two saturated carbon atoms attached to the nitrogen atom, further having a total of n carbon atoms, wherein at least 6 of the carbon atoms form an aromatic ring structure containing only carbon atoms, 8 or more hydrogen atoms, a total of one nitrogen atom, and no additional heteroatoms. For example, a heteroatom-unsubstituted C₇-C₁₀-aralkylamino has 7 to 10 carbon atoms. The term “heteroatom-unsubstituted Cₙ-aralkylamino” includes groups, having the structure —NHR, in which R is a heteroatom-unsubstituted Cₙ-aralkyl, as that term is defined above. An aralkylamino group includes diaralkylamino groups.

The term “heteroatom-substituted Cₙ-aralkylamino” refers to a radical, having a single nitrogen atom as the point of attachment, further having at least one or two saturated carbon atoms attached to the nitrogen atom, further having a total of n carbon atoms, 0, 1, or more than one hydrogen atom, at least one additional heteroatom, that is, in addition to the nitrogen atom at the point of attachment, wherein at least one of the carbon atom incorporated into an aromatic ring, further wherein each heteroatom is independently selected from the group consisting of N, O, F, Cl, Br, I, Si, P, and S. For example, a heteroatom-substituted C₇-C₁₀-aralkylamino has 7 to 10 carbon atoms. The term “heteroatom-substituted Cₙ-aralkylamino” includes groups, having the structure —NHR, in which R is a heteroatom-substituted Cₙ-aralkyl, as that term is defined above. The term “heteroatom-substituted aralkylamino” includes the term “heteroaralkylamino.”

The term “heteroatom-unsubstituted Cₙ-amido” refers to a radical, having a single nitrogen atom as the point of attachment, further having a carbonyl group attached via its carbon atom to the nitrogen atom, further having a linear or branched, cyclic or acyclic structure, further having a total of n carbon atoms, 1 or more hydrogen atoms, a total of one oxygen atom, a total of one nitrogen atom, and no additional heteroatoms. For example, a heteroatom-unsubstituted C₁-C₁₀-amido has 1 to 10 carbon atoms. The term “heteroatom-unsubstituted Cₙ-amido” includes groups, having the structure —NHR, in which R is a heteroatom-unsubstituted Cₙ-acyl, as that term is defined above. The term amido includes N-alkyl-amido, N-aryl-amido, N-aralkyl-amido, acylamino, alkylcarbonylamino, arylcarbonylamino, and ureido groups. The group, —NHCOCH₃, is an example of a heteroatom-unsubstituted amido group.

The term “heteroatom-substituted Cₙ-amido” refers to a radical, having a single nitrogen atom as the point of attachment, further having a carbonyl group attached via its carbon atom to the nitrogen atom, further having a linear or branched, cyclic or acyclic structure, further having a total of n aromatic or nonaromatic carbon atoms, 0, 1, or more than one hydrogen atom, at least one additional heteroatom in addition to the oxygen of the carbonyl group, wherein each additional heteroatom is independently selected from the group consisting of N, O, F, Cl, Br, I, Si, P, and S. For example, a heteroatom-substituted C₁-C₁₀-amido has 1 to 10 carbon atoms. The term “heteroatom-substituted Cₙ-amido” includes groups, having the structure —NHR, in which R is a heteroatom-unsubstituted Cₙ-acyl, as that term is defined above. The group, —NHCO₂CH₃, is an example of a heteroatom-substituted amido group.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

As used herein, a “chiral auxiliary” refers to a removable chiral group that is capable of influencing the stereoselectivity of a reaction. Persons of skill in the art are familiar with such compounds, and many are commercially available.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result.

The term “hydrate” when used as a modifier to a compound means that the compound has less than one (e.g., hemihydrate), one (e.g., monohydrate), or more than one (e.g., dehydrate) water molecules associated with each compound molecule, such as in solid forms of the compound.

As used herein, the term “IC₅₀” refers to an inhibitory dose which is 50% of the maximum response obtained.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dogs, cat, mouse, rat, guinea pig, or transgenic species thereof. In certain embodiments, the patient or subject is a primate. Non-limiting examples of human subjects are adults, juveniles, infants and fetuses.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylicacids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts Properties, and Use (P. H. Stahl & C. G. Wermuth eds., Verlag Helvetica Chimica Acta, 2002),

As used herein, “predominantly one enantiomer” means that a compound contains at least about 85% of one enantiomer, or more preferably at least about 90% of one enantiomer, or even more preferably at least about 95% of one enantiomer, or most preferably at least about 99% of one enantiomer. Similarly, the phrase “substantially free from other optical isomers” means that the composition contains at most about 15% of another enantiomer or diastereomer, more preferably at most about 10% of another enantiomer or diastereomer, even more preferably at most about 5% of another enantiomer or diastereomer, and most preferably at most about 1% of another enantiomer or diastereomer.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject of patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

The term “saturated” when referring to a atom means that the atom is connected to other atoms only by means of single bonds.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers.

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydro-pyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃), benzyloxycarbonyl, p-methoxybenzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethyloxycarbonyl groups (such as benzyloxycarbonyl and p-nitrobenzyloxycarbonyl), tert-butoxycarbonyl groups (—C(O)OC(CH₃)₃), and the like. Suitable peptide residues include peptide residues comprising two to five, and optionally amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethyloxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and halogenoethoxycarbonyl groups (such as β,β,β-trichloroethoxycarbonyl and β-iodoethoxycarbonyl).

“Therapeutically effective amount” means that amount which, when administered to an animal for treating a disease, is sufficient to effect such treatment for the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), (3) effecting any measurable decrease in a disease in a subject or patient that is experiencing or displaying the pathology or symptomatology of the disease, and/or (4) alleviating the symptoms of a disease in a subject or patient experiencing or displaying the pathology or symptomatology of the disease.

As used herein, the term “water soluble” means that the compound dissolves in water at least to the extent of 0.010 mole/liter or is classified as soluble according to literature precedence.

Other abbreviations used herein are as follows: DMSO, dimethyl sulfoxide; NO, nitric oxide; iNOS, inducible nitric oxide synthase; COX-2, cyclooxygenase-2; NGF, nerve growth factor; IBMX, isobutylmethylxanthine; FBS, fetal bovine serum; GPDH, glycerol 3-phosphate dehydrogenase; RXR, retinoid X receptor; TGF-β, transforming growth factor-β; IFNγ or IFN-γ, interferon-γ; LPS, bacterial endotoxic lipopolysaccharide; TNFα or TNF-α, tumor necrosis factor-α; IL-1β, interleukin-1β; GAPDH, glyceraldehyde-3-phosphate dehydrogenase; MTT, 3-[4,5-dimethylthiazol-2-yl]-2,5-diphenyltetrazolium bromide; TCA, trichloroacetic acid; HO-1, inducible heme oxygenase.

### III. Biological Activity of Synthetic Triterpenoids

Triterpenoids, biosynthesized in plants by the cyclization of squalene, are used for medicinal purposes in many Asian countries; and some, like ursolic and oleanolic acids, are known to be anti-inflammatory and anti-carcinogenic (Huang et al., 1994; Nishino et al., 1988). However, the biological activity of these naturally-occurring molecules is relatively weak, and therefore the synthesis of new analogs to enhance their potency was undertaken (Honda et al., 1997; Honda et al., 1998). Subsequent research has identified a number of synthetic triterpenoids (TPs) that have improved activity as compared to the naturally-occurring triterpenoids.

The ongoing efforts for the improvement of anti-inflammatory and antiproliferative activity of oleanolic and ursolic acid analogs led to the discovery of synthetic triterpenoids, such as 2-cyano-3,12-dioxooleane-1,9(11)-dien-28-oic acid (CDDO) and related compounds (e.g., CDDO-Me, TP-225, CDDO-Im) (Honda et al., 1997, 1998, 1999, 2000a, 2000b, 2002; Suh et al., 1998; 1999; 2003; Place et al., 2003; Liby et al., 2005). Triterpenoids have been shown to activate the Keap/Nrf2/ARE pathway a cytoprotective response is correlated to anti-inflammatory activity (Liby et al., 2005, Dinkova-Kostova et al., 2005; Thimmulappa et al., 2006; Yu and Kensler, 2005; Na and Surh, 2006). It has been reported that CDDO and its analogs form Michael adducts with thiol groups on cysteine residues of target proteins. Some of these such as Keap1 (Dinkova-Kostova et al., 2005), an inhibitor of the Nrf2 transcription factor that regulates the phase 2 cytoprotective response, and IκB kinase (Ahmad et al., 2006; Yore et al., 2006) have already been identified. Subsequent reports provided additional evidence consistent with CDDO-Me and CDDO-Im inhibiting IKKβ activity via binding to Cys179 (Ahmad et al., 2006; Yore et al., 2006). In a recent study concerning induction of cytoprotective genes through Keap 1-Nrf2-antioxidant response element (ARE) signaling, a structure activity evaluation of fifteen triterpenoids suggested that contributions of Michael acceptor groups on both the A and C rings, the nitrile group at C-2 of the A ring, and that substituents at C-17 affected pharmacodynamic action in vivo (Yates et al., 2007).

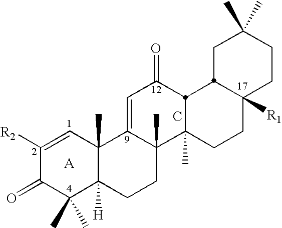

In general, synthetic triterpenoids (e.g., CDDO and derivatives thereof) have been shown useful in a variety of contexts. For example, CDDO-Me and CDDO-Im have been reported to modulate transforming growth factor-β (TGF-β)/Smad signaling in several types of cells (Suh et al., 2003; Minns et al., 2004; Mix et al., 2004). Also both have been reported to be potent inducers of heme-oxygenase-1 and Nrf2/ARE signaling (Liby et al., 2005).

Synthetic triterpenoid analogs of oleanolic acid have been shown to be powerful inhibitors of cellular inflammatory processes, such as the induction by IFN-γ of inducible nitric oxide synthase (iNOS) and of cyclooxygenase 2 in mouse macrophages. See Honda et al. (2000a); Honda et al. (2000b), and Honda et al. (2002), which are all incorporated herein by reference. The aberrant or excessive expression of either iNOS or cyclooxygenase-2 (COX-2) has been implicated in the pathogenesis of many disease processes. NO (nitric oxide) is a potent mutagen (Tamir and Tannebaum, 1996) and can also activate COX-2 (Salvemini et al., 1994). There is a marked increase in iNOS in rat colon tumors induced by the carcinogen, azoxymethane (Takahashi et al., 1997). The compounds and/or methods of this invention may be used to treat inflammatory conditions, such as sepsis, dermatitis, autoimmune disease, osteoarthritis, inflammatory pain and neuropathic pain.

Synthetic triterpenoids have been shown to affect include the blocking of NF-κB. It has been suggested that NF-κB activity may lead to enhancement of the cell cycle by its ability to activate cyclin D1 (Guttridge et al., 1999; Hinz et al., 1999; Joyce et al., 1999). Inhibition of IKK-driven NF-κB activation offers a strategy for treatment of different malignancies and can convert inflammation-induced tumor growth to inflammation-induced tumor regression. Luo et al., 2005. For example, Shishodia et al. (2006), reports that CDDO-Me modulates nuclear factor κB (NF-κB) activity and NF-κB-regulated gene expression. Using human leukemia cell lines and patient samples, it was shown that CDDO-Me potently inhibits both constitutive and inducible NF-κB activated by tumor necrosis factor (TNF), interleukin (IL)-1 β, phorbol ester, okadaic acid, hydrogen peroxide, lipopolysaccharide, and cigarette smoke. NF-κB suppression occurred through inhibition of IκBα kinase activation, IκBα phosphorylation, IκBα degradation, p65 phosphorylation, p65 nuclear translocation, and NF-κB-mediated reporter gene transcription. This inhibition was shown to correlate with suppression of NF-κB-dependent genes involved in antiapoptosis (IAP2, cFLIP, TRAF1, survivin, and bcl-2), proliferation (cyclin d1 and c-myc), and angiogenesis (VEGF, cox-2, and mmp-9). CDDO-Me was also shown to potentiate the cytotoxic effects of TNF and chemotherapeutic agents. In certain embodiments, the compounds and/or methods of this invention may be used to induce of Nrf2 and/or inhibit NF-κB.

Synthetic triterpenoids have also been shown to be potent inducers of the phase 2 response, that is elevation of NAD(P)H-quinone oxidoreductase and heme oxygenase 1 (HO-1), which protects cells against oxidative and electrophile stress. See Dinkova-Kostova et al., 2005. Induction of HO-1 has been shown to be therapeutic in animal models of many different diseases, including myocardial infarction, renal failure, transplant failure and rejection, stroke, cardiovascular disease, and autoimmune disease.

In animal models of many such conditions, stimulating expression of inducible heme oxygenase (HO-1) has been shown to have a significant therapeutic effect (e.g., Sacerdoti et al., 2005; Abraham & Kappas, 2005; Bach, 2006; Araujo et al., 2003; Liu et al., 2006; Ishikawa et al., 2001; Kruger et al., 2006; Satoh et al., 2006; Zhou et al., 2005; Morse and Choi, 2005; Morse and Choi, 2002.). This enzyme breaks free heme down into iron, carbon monoxide (CO), and biliverdin (which is subsequently converted to the potent antioxidant molecule, bilirubin). It was shown that at nanomolar concentrations, CDDO and CDDO-Im rapidly increase the expression of the cytoprotective heme oxygenase-1 (HO-1) enzyme in vitro and in vivo. See Liby et al. (2005). Transfection studies using a series of reporter constructs showed that activation of the human HO-1 promoter by the triterpenoids requires an antioxidant response element (ARE), a cyclic AMP response element, and an E Box sequence. Inactivation of one of these response elements alone was shown to partially reduce HO-1 induction, but mutations in all three sequences entirely eliminated promoter activity in response to the triterpenoids.

The compounds and/or methods of this invention may be used in treating subjects having a conditions caused by elevated levels of oxidative stress in one or more tissues. In some embodiments, the oxidative stress is accompanied by either acute or chronic inflammation. In some embodiments, the oxidative stress is caused by acute exposure to an external agent such as ionizing radiation or a cytotoxic chemotherapy agent (e.g., doxorubicin), by trauma or other acute tissue injury, by ischemia/reperfusion injury, by poor circulation or anemia, by localized or systemic hypoxia or hyperoxia, or by other abnormal physiological states such as hyperglycemia or hypoglycemia.

In another aspect, the compounds of the invention may be used in preventing or treating tissue damage or organ failure, acute and chronic, resulting from oxidative stress exacerbated by inflammation. Examples of diseases that fall in this category include: heart failure, liver failure, transplant failure and rejection, renal failure, pancreatitis, fibrotic lung diseases (cystic fibrosis and COPD, among others), diabetes (including complications), atherosclerosis, ischemia-reperfusion injury, glaucoma, stroke, autoimmune disease, autism, macular degeneration, and muscular dystrophy. For example, in the case of autism, studies suggest that increased oxidative stress in the central nervous system may contribute to the development of the disease (Chauhan and Chauhan, 2006). Evidence also links oxidative stress and inflammation to the development and pathology of many other disorders of the central nervous system, including psychiatric disorders such as psychosis, major depression, and bipolar disorder; seizure disorders such as epilepsy; pain and sensory syndromes such as migraine, neuropathic pain or tinnitus; and behavioral syndromes such as the attention deficit disorders. See, e.g., Dickerson et al., 2007; Hanson et al., 2005; Kendall-Tackett, 2007; Lencz et al., 2007; Dudhgaonkar et al., 2006; Lee et al., 2007; Morris et al., 2002; Ruster et al., 2005; McIver et al., 2005; Sarchielli et al., 2006; Kawakami et al., 2006; Ross et al., 2003, which are all incorporated by reference herein. For example, elevated levels of inflammatory cytokines, including TNF, interferon-γ, and IL-6, are associated with major mental illness (Dickerson et al., 2007). Microglial activation has also been linked to major mental illness. Therefore, downregulating inflammatory cytokines and inhibiting excessive activation of microglia could be beneficial in patients with schizophrenia, major depression, bipolar disorder, autism-spectrum disorders, and other neuropsychiatric disorders. Therefore, in another aspect, the invention provides compounds and/or methods using known oleanic derivatives that may be used in preventing or treating the above described disorders and other pathologies involving oxidative stress alone or oxidative stress exacerbated by inflammation. Treatment may be administered preventively, in advance of a predictable state of oxidative stress (e.g., organ transplantation or the administration of radiation therapy to a cancer patient), or it may be administered therapeutically in settings involving established oxidative stress and inflammation.

Inflammatory, oxidative, or immune mechanisms have also been implicated in the pathogenesis of Alzheimer's disease (AD), Parkinson's disease (PD), amyotrophic lateral sclerosis (ALS), and MS (Bagasra et al., 1995; McGeer and McGeer, 1995; Simonian and Coyle, 1996; Kaltschmidt et al., 1997). Both reactive astrocytes and activated microglia have been implicated in causation of neurodegenerative disease (NDD) and neuroinflammatory disease (NID); there has been a particular emphasis on microglia as cells that synthesize both NO and prostaglandins as products of the respective enzymes, iNOS and COX-2. De novo formation of these enzymes may be driven by inflammatory cytokines such as interferon-γ or interleukin-1. In turn, excessive production of NO may lead to inflammatory cascades and/or oxidative damage in cells and tissues of many organs, including neurons and oligodendrocytes of the nervous system, with consequent manifestations in AD and MS, and possible PD and ALS (Coyle and Puttfarcken, 1993; Beal, 1996; Merrill and Benvenist, 1996; Simonian and Coyle, 1996; Vodovotz et al., 1996). Epidemiologic data indicate that chronic use of NSAID's which block synthesis of prostaglandins from arachidonate, markedly lower the risk for development of AD (McGeer et al., 1996; Stewart et al., 1997). Thus, agents that block formation of NO and prostaglandins, may be used in approaches to prevention and treatment of NDD.

The compounds of the present invention may be used in preventing and/or treating diseases or disorders whose pathology involves oxidative stress, inflammation, and/or dysregulation of inflammatory signaling pathways. In some variations, the diseases or disorders can be characterized by overexpression of inducible nitric oxide synthase (iNOS) and/or inducible cyclooxygenase (COX-2) in affected tissues. In some variations, the diseases or disorders can be characterized by overproduction of reactive oxygen species (ROS) or reactive nitrogen species (RNS) such as superoxide, hydrogen peroxide, nitric oxide or peroxynitrite in affected tissues. In some variations, the disease or disorder is characterized by excessive production of inflammatory cytokines or other inflammation-related proteins such as TNFα, IL-6, IL-1, IL-8, ICAM-1, VCAM-1, and VEGF. Such diseases or disorders may, in some embodiments, involve undesirable proliferation of certain cells, as in the case of cancer (e.g., solid tumors, leukemias, myelomas, lymphomas, and other cancers), fibrosis associated with organ failure, or excessive scarring. Non limiting examples of the disease or disorder include: lupus, rheumatoid arthritis, juvenile-onset diabetes, multiple sclerosis, psoriasis, and Crohn's disease. Further non-limiting examples include cardiovascular diseases, such as atherosclerosis, heart failure, myocardial infarction, acute coronary syndrome, restenosis following vascular surgery, hypertension, and vasculitis; neurodegenerative or neuromuscular diseases such as Alzheimer's disease, Parkinson's disease, Huntington's disease, ALS, and muscular dystrophy; neurological disorders such as epilepsy and dystonia; neuropsychiatric conditions such as major depression, bipolar disorder, post-traumatic stress disorder, schizophrenia, anorexia nervosa, ADHD, and autism-spectrum disorders; retinal diseases such as macular degeneration, diabetic retinopathy, glaucoma, and retinitis; chronic and acute pain syndromes, including inflammatory and neuropathic pain; hearing loss and tinnitus; diabetes and complications of diabetes, including metabolic syndrome, diabetic nephropathy, diabetic neuropathy and diabetic ulcers; respiratory diseases such as asthma, chronic obstructive pulmonary disease, acute respiratory distress syndrome, and cystic fibrosis; inflammatory bowel diseases; osteoporosis, osteoarthritis, and other degenerative conditions of bone and cartilage; acute or chronic organ failure, including renal failure, liver failure (including cirrhosis and hepatitis), and pancreatitis; ischemia-reperfusion injury associated with thrombotic or hemorrhagic stroke, subarachnoid hemorrhage, cerebral vasospasm, myocardial infarction, shock, or trauma; complications of organ or tissue transplantation including acute or chronic transplant failure or rejection and graft-versus-host disease; skin diseases including atopic dermatitis and acne; sepsis and septic shock; excessive inflammation associated with infection, including respiratory inflammation associated with influenza and upper respiratory infections; mucositis associated with cancer therapy, including radiation therapy or chemotherapy; and severe burns.

### IV. Synthetic Methods

Compounds of the present invention as well as the known compound used in the methods provided by the present invention can be made using the methods outlined below or by modifications or optimizations thereof using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

In addition to providing new methods of treatment of the methyl amide of CDDO (CDDO-MA), the synthesis of which was reported in (Honda et al., 2002), the invention provides additional CDDO amide derivatives, such as the ethyl amide (CDDO-EA), as well fluorinated amide derivative of CDDO, such as the 2,2,2-trifluoroethyl amide derivative of CDDO (CDDO-TFEA).

Synthetic triterpenoids corresponding to formulas Ia, Ib, IIa, IIb, III and IV can be prepared according to the methods taught by Honda et al. (1998), Honda et al. (2000b), Honda et al., (2002) and Yates et al. (2007), which are all incorporated herein by reference.

The synthesis of CDDO-MA is discussed in Honda et al. (2002), which is incorporated herein by reference. The syntheses of CDDO-EA and CDDO-TFEA are presented in Yates et al. (2007), which is incorporated herein by reference, and shown in the Scheme 1 below.

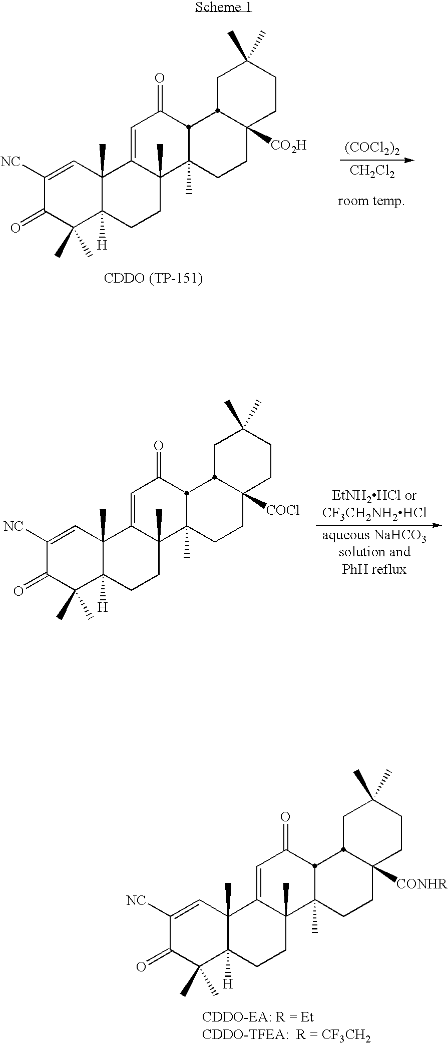

### V. Penetration of Blood Brain Barrier

The treatment of neurodegenerative diseases and other diseases affecting the central nervous system (CNS), typically requires an agent that is able to penetrate the blood brain barrier (BBB). Similarly, treatment of spinal cord diseases and injuries typically requires an agent that is able to penetrate the blood-spinal cord barrier (BSCB). For example, because a majority of chemotherapy agents are unable to penetrate the BBB and cannot reach effective concentrations in the brain, few effective agents for the treatment of brain cancer are available. Primary brain cancers such as glioblastoma multiforme are among the deadliest cancers due to their rapid progression and the lack of effective treatments. Brain metastases arising from common primary cancers such as breast and lung cancer are also a major source of morbidity and mortality, not least because agents that are effective in treating these tumors outside the CNS cannot cross the BBB. Brain metastases, therefore, are sheltered from exposure to agents that otherwise would effectively inhibit their growth.

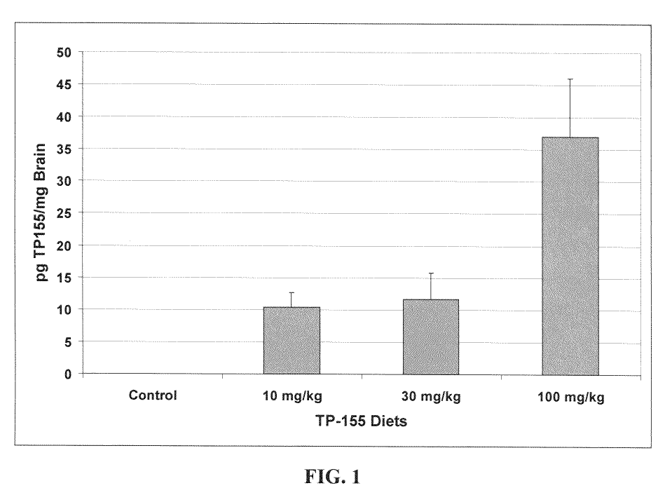

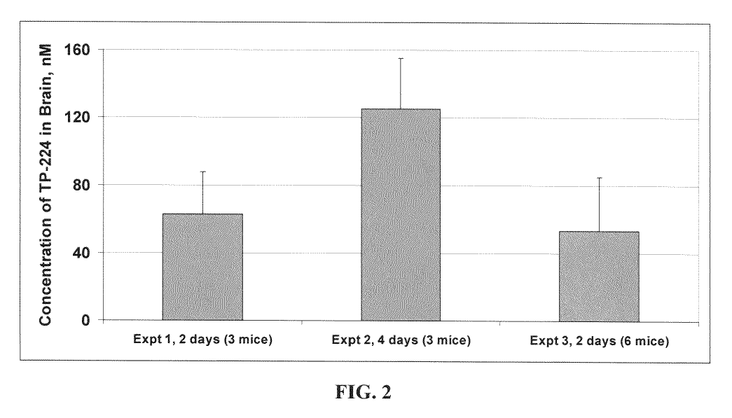

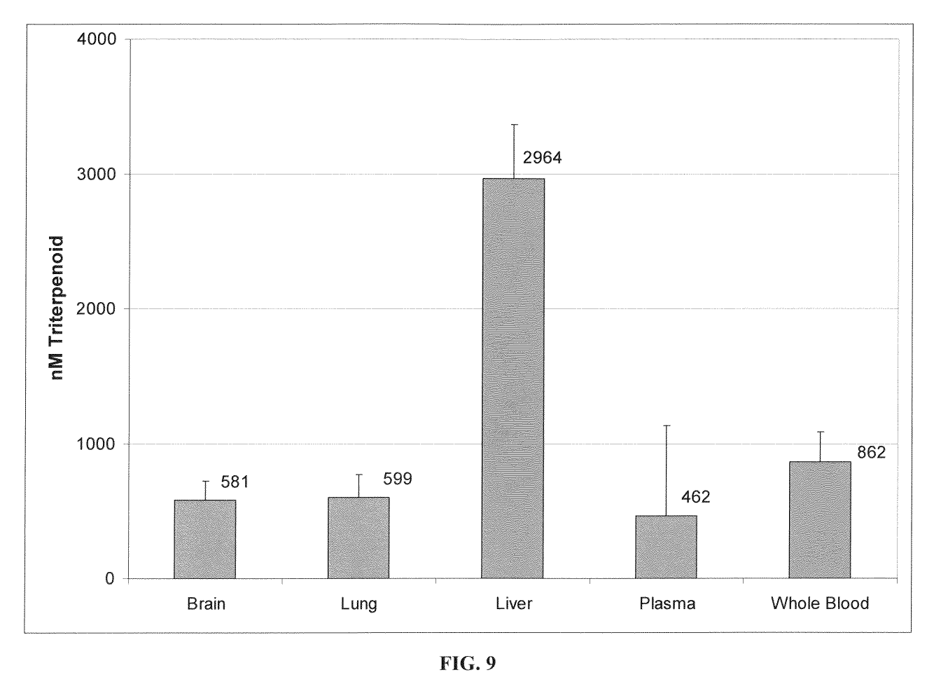

The results presented in Example 2 and FIGS. 1-9 demonstrate that the synthetic triterpenoids studied as part of this invention are able to cross the blood-brain barrier and reach significant concentrations in the brain. For example, FIG. 1 shows that CDDO-Me is able to reach appreciable levels in the brain after one week of feeding (100 mg/kg diet). The levels measured are comparable to those reached by TP-224 (CDDO-MA) after only 2 days of feeding at a higher dose. Furthermore, the brain levels achieved after oral administration of CDDO-TFEA (TP-500) are comparable to those achieved in other tissue compartments such as lung (FIG. 9).

### VI. Use of Triterpenoids for the Treatment of Multiple Sclerosis

Multiple sclerosis (MS) is known to be an inflammatory condition of the central nervous system (Williams et al., 1994; Merrill and Benvenist, 1996; Genain and Nauser, 1997). Based on the results presented in this application, the compounds and methods of this invention are expected to have substantial utility for treating multiple sclerosis (MS) in subjects. For example, the inventors show that CDDO-TFEA and CDDO-Me induce full recovery of mice in a rapidly progressive experimental autoimmune encephalomyelitis (EAE) model (MOG induced EAE).

Experimental Autoimmune Encephalomyelitis (EAE), also called Experimental Allergic Encephalomyelitis, is an animal model of Multiple Sclerosis. EAE is not multiple sclerosis, nor is it a single disease in a single species, but its different forms resemble the various forms and stages of MS very closely in a large number of ways. EAE, like MS, is an inflammatory, demyelinating disorder of the central nervous system. The primary difference between EAE and MS is that EAE must be induced in animals while MS occurs spontaneously in humans. Animals are injected with the whole or parts of various proteins that make up myelin, the insulating sheath that surrounds nerve cells (neurons). These proteins induce an autoimmune response in the animals, causing the animal's immune system to mount an attack on its own myelin as a result of exposure to the injection. The animals develop a disease process that closely resembles MS in humans.

Several proteins or parts of proteins (antigens) are used to induce EAE including: Myelin Basic Protein (MBP), Proteolipid Protein (PLP), and Myelin Oligodendrocyte Glycoprotein (MOG). For example, MOG induced EAE is often considered to be a better model of primary progressive MS, while PLP induced MS is often considered to be a better model of relapsing remitting MS. MOG typically induces chronic paralytic EAE, while PLP typically induces relapsing-remitting EAE. The various EAE models continue to provide valuable information to our understanding and treatment of MS, and results obtained in both EAE models have been shown to be very relevant to understanding the effectiveness of treatments for many subtypes of MS. See Gold et al., (2006); Juedes et al., (2000); Owens (2006); Virley (2005), which are all incorporated herein by reference.

### VII. Use of Triterpenoids for the Treatment Other Diseases Associated with Inflammation and/or Oxidative Stress

Inflammation is a biological process that provides resistance to infectious or parasitic organisms and the repair of damaged tissue. Inflammation is commonly characterized by localized vasodilation, redness, swelling, and pain, the recruitment of leukocytes to the site of infection or injury, production of inflammatory cytokines such as TNF-α and IL-1, and production of reactive oxygen or nitrogen species such as hydrogen peroxide, superoxide and peroxynitrite. In later stages of inflammation, tissue remodeling, angiogenesis, and scar formation (fibrosis) may occur as part of the wound healing process. Under normal circumstances, the inflammatory response is regulated and temporary and is resolved in an orchestrated fashion once the infection or injury has been dealt with adequately. However, acute inflammation can become excessive and life-threatening if regulatory mechanisms fail. Alternatively, inflammation can become chronic and cause cumulative tissue damage or systemic complications.

Many serious and intractable human diseases involve dysregulation of inflammatory processes, including diseases such as cancer, atherosclerosis, and diabetes, which were not traditionally viewed as inflammatory conditions. In the case of cancer, the inflammatory processes is associated with tumor formation, progression, metastasis, and resistance to therapy. Atherosclerosis, long viewed as a disorder of lipid metabolism, is now understood to be primarily an inflammatory condition, with activated macrophages playing an important role in the formation and eventual rupture of atherosclerotic plaques. Activation of inflammatory signaling pathways has also been shown to play a role in the development of insulin resistance, as well as in the peripheral tissue damage associated with diabetic hyperglycemia. Excessive production of reactive oxygen species and reactive nitrogen species such as superoxide, hydrogen peroxide, nitric oxide, and peroxynitrite is a hallmark of inflammatory conditions. Evidence of dysregulated peroxynitrite production has been reported in a wide variety of diseases (Szabo et al., 2007; Schulz et al., 2008; Forstermann, 2006; Pall, 2007).

Autoimmune diseases such as rheumatoid arthritis, lupus, psoriasis, and multiple sclerosis involve inappropriate and chronic activation of inflammatory processes in affected tissues, arising from dysfunction of self vs. non-self recognition and response mechanisms in the immune system. In neurodegenerative diseases such as Alzheimer's and Parkinson's diseases, neural damage is correlated with activation of microglia and elevated levels of pro-inflammatory proteins such as inducible nitric oxide synthase (iNOS). Chronic organ failure such as renal failure, heart failure, and chronic obstructive pulmonary disease is closely associated with the presence of chronic oxidative stress and inflammation, leading to the development of fibrosis and eventual loss of organ function.

Many other disorders involve oxidative stress and inflammation in affected tissues, including inflammatory bowel disease; inflammatory skin diseases; mucositis related to radiation therapy and chemotherapy; eye diseases such as uveitis, glaucoma, macular degeneration, and various forms of retinopathy; transplant failure and rejection; ischemia-reperfusion injury; chronic pain; degenerative conditions of the bones and joints including osteoarthritis and osteoporosis; asthma and cystic fibrosis; seizure disorders; and neuropsychiatric conditions including schizophrenia, depression, bipolar disorder, post-traumatic stress disorder, attention deficit disorders, autism-spectrum disorders, and eating disorders such as anorexia nervosa. Dysregulation of inflammatory signaling pathways is believed to be a major factor in the pathology of muscle wasting diseases including muscular dystrophy and various forms of cachexia.

A variety of life-threatening acute disorders also involve dysregulated inflammatory signaling, including acute organ failure involving the pancreas, kidneys, liver, or lungs, myocardial infarction or acute coronary syndrome, stroke, septic shock, trauma, severe burns, and anaphylaxis.

Many complications of infectious diseases also involve dysregulation of inflammatory responses. Although an inflammatory response can kill invading pathogens, an excessive inflammatory response can also be quite destructive and in some cases can be a primary source of damage in infected tissues. Furthermore, an excessive inflammatory response can also lead to systemic complications due to overproduction of inflammatory cytokines such as TNF-α and IL-1. This is believed to be a factor in mortality arising from severe influenza, severe acute respiratory syndrome, and sepsis.

These properties are relevant to the treatment of a wide array of diseases involving oxidative stress and dysregulation of inflammatory processes including cancer, mucositis resulting from radiation therapy or chemotherapy, autoimmune diseases, cardiovascular diseases, ischemia-reperfusion injury, acute and chronic organ failure including renal failure and heart failure, respiratory diseases, diabetes and complications of diabetes, severe allergies, transplant rejection, graft-versus-host disease, neurodegenerative diseases, diseases of the eye and retina, acute and chronic pain, degenerative bone diseases including osteoarthritis and osteoporosis, inflammatory bowel diseases, dermatitis and other skin diseases, cardiovascular diseases including atherosclerosis, sepsis, burns, seizure disorders, and neuropsychiatric disorders.

In another aspect, compounds of the invention may be used for treating a subject having a condition caused by elevated levels of oxidative stress in one or more tissues. Oxidative stress results from abnormally high or prolonged levels of reactive oxygen species such as superoxide, hydrogen peroxide, nitric oxide, and peroxynitrite (formed by the reaction of nitric oxide and superoxide). The oxidative stress may be accompanied by either acute or chronic inflammation. The oxidative stress may be caused by mitochondrial dysfunction, by activation of immune cells such as macrophages and neutrophils, by acute exposure to an external agent such as ionizing radiation or a cytotoxic chemotherapy agent (e.g., doxorubicin), by trauma or other acute tissue injury, by ischemia/reperfusion, by poor circulation or anemia, by localized or systemic hypoxia or hyperoxia, by elevated levels of inflammatory cytokines and other inflammation-related proteins, and/or by other abnormal physiological states such as hyperglycemia or hypoglycemia.

In animal models of many such conditions, stimulating expression of inducible heme oxygenase (HO-1) has been shown to have a significant therapeutic effect including models of myocardial infarction, renal failure, transplant failure and rejection, stroke, cardiovascular disease, and autoimmune disease (e.g., Sacerdoti et al., 2005; Abraham & Kappas, 2005; Bach, 2006; Araujo et al., 2003; Liu et al., 2006; Ishikawa et al., 2001; Kruger et al., 2006; Satoh et al., 2006; Zhou et al., 2005; Morse and Choi, 2005; Morse and Choi, 2002). This enzyme breaks free heme down into iron, carbon monoxide (CO), and biliverdin (which is subsequently converted to the potent antioxidant molecule, bilirubin).

In another aspect, compounds of this invention may be used in preventing or treating tissue damage or organ failure, acute and chronic, resulting from oxidative stress exacerbated by inflammation. Examples of diseases that fall in this category include: heart failure, liver failure, transplant failure and rejection, renal failure, pancreatitis, fibrotic lung diseases (cystic fibrosis and COPD, among others), diabetes (including complications), atherosclerosis, ischemia-reperfusion injury, glaucoma, stroke, autoimmune disease, autism, macular degeneration, and muscular dystrophy. For example, in the case of autism, studies suggest that increased oxidative stress in the central nervous system may contribute to the development of the disease (Chauhan and Chauhan, 2006).

Evidence also links oxidative stress and inflammation to the development and pathology of many other disorders of the central nervous system, including psychiatric disorders such as psychosis, major depression, and bipolar disorder; seizure disorders such as epilepsy; pain and sensory syndromes such as migraine, neuropathic pain or tinnitus; and behavioral syndromes such as the attention deficit disorders. See, e.g., Dickerson et al., 2007; Hanson et al., 2005; Kendall-Tackett, 2007; Lencz et al., 2007; Dudhgaonkar et al., 2006; Lee et al., 2007; Morris et al., 2002; Ruster et al., 2005; McIver et al., 2005; Sarchielli et al., 2006; Kawakami et al., 2006; Ross et al., 2003, which are all incorporated by reference herein. For example, elevated levels of inflammatory cytokines, including TNF, interferon-γ, and IL-6, are associated with major mental illness (Dickerson et al., 2007). Microglial activation has also been linked to major mental illness. Therefore, downregulating inflammatory cytokines and inhibiting excessive activation of microglia could be beneficial in patients with schizophrenia, major depression, bipolar disorder, autism-spectrum disorders, and other neuropsychiatric disorders.

Accordingly, in pathologies involving oxidative stress alone or oxidative stress exacerbated by inflammation, treatment may comprise administering to a subject a therapeutically effective amount of a compound of this invention, such as those described above or throughout this specification. Treatment may be administered preventively, in advance of a predictable state of oxidative stress (e.g., organ transplantation or the administration of radiation therapy to a cancer patient), or it may be administered therapeutically in settings involving established oxidative stress and inflammation.

The compounds of the invention may be generally applied to the treatment of inflammatory conditions, such as sepsis, dermatitis, autoimmune disease and osteoarthritis. In one aspect, the compounds of this invention may be used to treat inflammatory pain and/or neuropathic pain, for example, by inducing Nrf2 and/or inhibiting NF-κB.

In one aspect, the compounds of the invention may be used to function as antioxidant inflammation modulators (AIMs) having potent anti-inflammatory properties that mimic the biological activity of cyclopentenone prostaglandins (cyPGs). In one embodiment, the compounds of the invention may be used to control the production of pro-inflammatory cytokines by selectively targeting regulatory cysteine residues (RCRs) on proteins that regulate the transcriptional activity of redox-sensitive transcription factors. Activation of RCRs by cyPGs or AIMs has been shown to initiate a pro-resolution program in which the activity of the antioxidant and cytoprotective transcription factor Nrf2 is potently induced, and the activities of the pro-oxidant and pro-inflammatory transcription factors NF-κB and the STATs are suppressed. This increases the production of antioxidant and reductive molecules (e.g., NQO1, HO-1, SOD 1, and/or γ-GCS) and/or decreases oxidative stress and the production of pro-oxidant and pro-inflammatory molecules (e.g., iNOS, COX-2, and/or TNF-α).

In some embodiments, the compounds of the invention may be used in the treatment and prevention of diseases such as cancer, inflammation, Alzheimer's disease, Parkinson's disease, multiple sclerosis, autism, amyotrophic lateral sclerosis, autoimmune diseases such as rheumatoid arthritis, lupus, and MS, inflammatory bowel disease, all other diseases whose pathogenesis is believed to involve excessive production of either nitric oxide or prostaglandins, and pathologies involving oxidative stress alone or oxidative stress exacerbated by inflammation.

Another aspect of inflammation is the production of inflammatory prostaglandins such as prostaglandin E. These molecules promote vasodilation, plasma extravasation, localized pain, elevated temperature, and other symptoms of inflammation. The inducible form of the enzyme COX-2 is associated with their production, and high levels of COX-2 are found in inflamed tissues. Consequently, inhibition of COX-2 may relieve many symptoms of inflammation and a number of important anti-inflammatory drugs (e.g., ibuprofen and celecoxib) act by inhibiting COX-2 activity. Recent research, however, has demonstrated that a class of cyclopentenone prostaglandins (cyPGs) (e.g., 15-deoxy prostaglandin J2, a.k.a. PGJ2) plays a role in stimulating the orchestrated resolution of inflammation. COX-2 is also associated with the production of cyclopentenone prostaglandins. Consequently, inhibition of COX-2 may interfere with the full resolution of inflammation, potentially promoting the persistence of activated immune cells in tissues and leading to chronic, “smoldering” inflammation. This effect may be responsible for the increased incidence of cardiovascular disease in patients using selective COX-2 inhibitors for long periods of time.

In one aspect, the compounds of the invention may be used to control the production of pro-inflammatory cytokines within the cell by selectively activating regulatory cysteine residues (RCRs) on proteins that regulate the activity of redox-sensitive transcription factors. Activation of RCRs by cyPGs has been shown to initiate a pro-resolution program in which the activity of the antioxidant and cytoprotective transcription factor Nrf2 is potently induced and the activities of the pro-oxidant and pro-inflammatory transcription factors NF-κB and the STATs are suppressed. In some embodiments, this increases the production of antioxidant and reductive molecules (NQO1, HO-1, SOD1, γ-GCS) and decreases oxidative stress and the production of pro-oxidant and pro-inflammatory molecules (iNOS, COX-2, TNF-α). In some embodiments, the compounds of this invention may cause the cells that host the inflammatory event to revert to a non-inflammatory state by promoting the resolution of inflammation and limits excessive tissue damage to the host.

A. Cancer

The levels of iNOS and COX-2 are elevated in certain cancers and have been implicated in carcinogenesis and COX-2 inhibitors have been shown to reduce the incidence of primary colonic adenomas in humans (Rostom et al., 2007; Brown and DuBois, 2005; Crowel et al., 2003). iNOS is expressed in myeloid-derived tumor suppressor cells (MDSCs) (Angulo et al., 2000) and COX-2 activity in cancer cells has been shown to result in the production of prostaglandin E₂ (PGE₂), which has been shown to induce the expression of arginase in MDSCs (Sinha et al., 2007). Arginase and iNOS are enzymes that utilize L-arginine as a substrate and produce L-ornithine and urea, and L-citrulline and NO, respectively. The depletion of arginine from the tumor microenvironment by MDSCs, combined with the production of NO and peroxynitrite has been shown to inhibit proliferation and induce apoptosis of T cells (Bronte et al., 2003). Inhibition of COX-2 and iNOS has been shown to reduce the accumulation of MDSCs, restore cytotoxic activity of tumor-associated T cells, and delay tumor growth (Sinha et al., 2007; Mazzoni et al., 2002; Zhou et al., 2007).

Inhibition of the NF-κB and JAK/STAT signaling pathways has been implicated as a strategy to inhibit proliferation of cancer epithelial cells and induce their apoptosis. Activation of STAT3 and NF-κB has been shown to result in suppression of apoptosis in cancer cells, and promotion of proliferation, invasion, and metastasis. Many of the target genes involved in these processes have been shown to be transcriptionally regulated by both NF-κB and STAT3 (Yu et al., 2007).

In addition to their direct roles in cancer epithelial cells, NF-κB and STAT3 also have important roles in other cells found within the tumor microenvironment. Experiments in animal models have demonstrated that NF-κB is required in both cancer cells and hematopoeitic cells to propagate the effects of inflammation on cancer initiation and progression (Greten et al., 2004). NF-κB inhibition in cancer and myeloid cells reduces the number and size, respectively, of the resultant tumors. Activation of STAT3 in cancer cells results in the production of several cytokines (IL-6, IL-10) which suppress the maturation of tumor-associated dendritic cells. Furthermore, STAT3 is activated by these cytokines in the dendritic cells themselves. Inhibition of STAT3 in mouse models of cancer restores DC maturation, promotes antitumor immunity, and inhibits tumor growth (Kortylewski et al., 2005).

Further, compounds of the present invention may be used in inducing apoptosis in tumor cells, inducing cell differentiation, inhibiting cancer cell proliferation, inhibiting inflammatory response, and/or functioning in a chemopreventative capacity. For example, the invention provides new compounds that have one or more of the following properties: (1) the ability to induce apoptosis and differentiate both malignant and non-malignant cells, (2) activity at sub-micromolar or nanomolar levels as an inhibitor of proliferation of many malignant or premalignant cells, (3) the ability to suppress the de novo synthesis of the inflammatory enzyme inducible nitric oxide synthase (iNOS), (4) the ability to inhibit NF-κB activation, and (5) the ability to induce the expression of heme oxygenase-1 (HO-1).

B. Neuroinflammation

Neuroinflammation encapsulates the idea that microglial and astrocytic responses and actions in the central nervous system have a fundamentally inflammation-like character, and that these responses are central to the pathogenesis and progression of a wide variety of neurological disorders. This idea originated in the field of Alzheimer's disease (Griffin et al., 1989; Rogers et al., 1988), where it has revolutionized our understanding of this disease (Akiyama et al., 2000). These ideas have been extended to other neurodegenerative diseases (Eikelenboom et al., 2002; Ishizawa and Dickson, 2001), to ischemic/toxic diseases (Gehrmann et al., 1995; Touzani et al., 1999), to tumor biology (Graeber et al., 2002) and even to normal brain development.

Neuroinflammation incorporates a wide spectrum of complex cellular responses that include activation of microglia and astrocytes and induction of cytokines, chemokines, complement proteins, acute phase proteins, oxidative injury, and related molecular processes. These events may have detrimental effects on neuronal function, leading to neuronal injury, further glial activation, and ultimately neurodegeneration.

Based on experimental results obtained, including those presented in this application, the compounds and methods of this invention may be used for treating patients with neuroinflammation.

C. Treatment of Renal Failure

Another aspect of the present invention concerns new methods and compounds for the treatment and prevention of renal disease. Renal failure, resulting in inadequate clearance of metabolic waste products from the blood and abnormal concentrations of electrolytes in the blood, is a significant medical problem throughout the world, especially in developed countries. Diabetes and hypertension are among the most important causes of chronic renal failure (CKD), but it is also associated with other conditions such as lupus. Acute renal failure may arise from exposure to certain drugs (e.g., acetaminophen) or toxic chemicals, or from ischemia-reperfusion injury associated with shock or surgical procedures such as transplantation, and may result in chronic renal failure. In many patients, renal failure advances to a stage in which the patient requires regular dialysis or kidney transplantation to continue living. Both of these procedures are highly invasive and associated with significant side effects and quality of life issues. Although there are effective treatments for some complications of renal failure, such as hyperparathyroidism and hyperphosphatemia, no available treatment has been shown to halt or reverse the underlying progression of renal failure. Thus, agents that can improve compromised renal function would represent a significant advance in the treatment of renal failure.

Inflammation contributes significantly to the pathology of CKD. There is also a strong mechanistic link between oxidative stress and renal dysfunction. The NF-κB signaling pathway plays an important role in the progression of CKD as NF-κB regulates the transcription of MCP-1, a chemokine that is responsible for the recruitment of monocytes/macrophages resulting in an inflammatory response that ultimately injures the kidney (Wardle, 2001). The Keap 1/Nrf2/ARE pathway controls the transcription of several genes encoding antioxidant enzymes, including heme oxygenase-1 (HO-1). Ablation of the Nrf2 gene in female mice results in the development of lupus-like glomerular nephritis (Yoh et al., 2001). Furthermore, several studies have demonstrated that HO-1 expression is induced in response to renal damage and inflammation and that this enzyme and its products—bilirubin and carbon monoxide—play a protective role in the kidney (Nath et al., 2006).

The glomerulus and the surrounding Bowman's capsule constitute the basic functional unit of the kidney. Glomerular filtration rate (GFR) is the standard measure of renal function. Creatinine clearance is commonly used to measure GFR. However, the level of serum creatinine is commonly used as a surrogate measure of creatinine clearance. For instance, excessive levels of serum creatinine are generally accepted to indicate inadequate renal function and reductions in serum creatinine over time are accepted as an indication of improved renal function. Normal levels of creatinine in the blood are approximately 0.6 to 1.2 milligrams (mg) per deciliter (dl) in adult males and 0.5 to 1.1 milligrams per deciliter in adult females.

Acute kidney injury (AKI) can occur following ischemia-reperfusion, treatment with certain pharmacological agents such as cisplatin and rapamycin, and intravenous injection of radiocontrast media used in medical imaging. As in CKD, inflammation and oxidative stress contribute to the pathology of AKI. The molecular mechanisms underlying radiocontrast-induced nephropathy (RCN) are not well understood; however, it is likely that a combination of events including prolonged vasoconstriction, impaired kidney autoregulation, and direct toxicity of the contrast media all contribute to renal failure (Tumlin et al., 2006). Vasoconstriction results in decreased renal blood flow and causes ischemia-reperfusion and the production of reactive oxygen species. HO-1 is strongly induced under these conditions and has been demonstrated to prevent ischemia-reperfusion injury in several different organs, including the kidney (Nath et al., 2006). Specifically, induction of HO-1 has been shown to be protective in a rat model of RCN (Goodman et al., 2007). Reperfusion also induces an inflammatory response, in part though activation of NF-κB signaling (Nichols, 2004). Targeting NF-κB has been proposed as a therapeutic strategy to prevent organ damage (Zingarelli et al., 2003).

Based on experimental results obtained, including those presented in this application, the compounds of this invention may be used for treating patients with renal failure.

D. Cardiovascular Disease

Cardiovascular (CV) disease is among the most important causes of mortality worldwide, and is the leading cause of death in many developed nations. The etiology of CV disease is complex, but the majority of causes are related to inadequate or completely disrupted supply of blood to a critical organ or tissue. Frequently such a condition arises from the rupture of one or more atherosclerotic plaques, which leads to the formation of a thrombus that blocks blood flow in a critical vessel. Such thrombosis is the principal cause of heart attacks, in which one or more of the coronary arteries is blocked and blood flow to the heart itself is disrupted. The resulting ischemia is highly damaging to cardiac tissue, both from lack of oxygen during the ischemic event and from excessive formation of free radicals after blood flow is restored (a phenomenon known as ischemia-reperfusion injury). Similar damage occurs in the brain during a thrombotic stroke, when a cerebral artery or other major vessel is blocked by thrombosis. Hemorrhagic strokes, in contrast, involve rupture of a blood vessel and bleeding into the surrounding brain tissue. This creates oxidative stress in the immediate area of the hemorrhage, due to the presence of large amounts of free heme and other reactive species, and ischemia in other parts of the brain due to compromised blood flow. Subarachnoid hemorrhage, which is frequently accompanied by cerebral vasospasm, also causes ischemia/reperfusion injury in the brain.

Alternatively, atherosclerosis may be so extensive in critical blood vessels that stenosis (narrowing of the arteries) develops and blood flow to critical organs (including the heart) is chronically insufficient. Such chronic ischemia can lead to end-organ damage of many kinds, including the cardiac hypertrophy associated with congestive heart failure.

Atherosclerosis, the underlying defect leading to many forms of cardiovascular disease, occurs when a physical defect or injury to the lining (endothelium) of an artery triggers an inflammatory response involving the proliferation of vascular smooth muscle cells and the infiltration of leukocytes into the affected area. Ultimately, a complicated lesion known as an atherosclerotic plaque may form, composed of the above-mentioned cells combined with deposits of cholesterol-bearing lipoproteins and other materials.

Pharmaceutical treatments for cardiovascular disease include preventive treatments, such as the use of drugs intended to lower blood pressure or circulating levels of cholesterol and lipoproteins, as well as treatments designed to reduce the adherent tendencies of platelets and other blood cells (thereby reducing the rate of plaque progression and the risk of thrombus formation). More recently, drugs such as streptokinase and tissue plasminogen activator have been introduced and are used to dissolve the thrombus and restore blood flow. Surgical treatments include coronary artery bypass grafting to create an alternative blood supply, balloon angioplasty to compress plaque tissue and increase the diameter of the arterial lumen, and carotid endarterectomy to remove plaque tissue in the carotid artery. Such treatments, especially balloon angioplasty, may be accompanied by the use of stents, expandable mesh tubes designed to support the artery walls in the affected area and keep the vessel open. Recently, the use of drug-eluting stents has become common in order to prevent post-surgical restenosis (renarrowing of the artery) in the affected area. These devices are wire stents coated with a biocompatible polymer matrix containing a drug that inhibits cell proliferation (e.g., paclitaxel or rapamycin). The polymer allows a slow, localized release of the drug in the affected area with minimal exposure of non-target tissues. Despite the significant benefits offered by such treatments, mortality from cardiovascular disease remains high and significant unmet needs in the treatment of cardiovascular disease remain.

As noted above, induction of HO-1 has been shown to be beneficial in a variety of models of cardiovascular disease, and low levels of HO-1 expression have been clinically correlated with elevated risk of CV disease. Compounds of the invention, therefore, may be used in treating or preventing a variety of cardiovascular disorders including but not limited to atherosclerosis, hypertension, myocardial infarction, chronic heart failure, stroke, subarachnoid hemorrhage, and restenosis.

Based on experimental results obtained, including those presented in this application, the compounds of this invention may be used for treating patients with cardiovascular disease.

E. Diabetes

Diabetes is a complex disease characterized by the body's failure to regulate circulating levels of glucose. This failure may result from a lack of insulin, a peptide hormone that regulates the both the production and absorption of glucose in various tissues. Deficient insulin compromises the ability of muscle, fat, and other tissues to absorb glucose properly, leading to hyperglycemia (abnormally high levels of glucose in the blood). Most commonly, such insulin deficiency results from inadequate production in the islet cells of the pancreas. In the majority of cases this arises from autoimmune destruction of these cells, a condition known as type 1 or juvenile-onset diabetes, but may also be due to physical trauma or some other cause.

Diabetes may also arise when muscle and fat cells become less responsive to insulin and do not absorb glucose properly, resulting in hyperglycemia. This phenomenon is known as insulin resistance, and the resulting condition is known as Type 2 diabetes. Type 2 diabetes, the most common type, is highly associated with obesity and hypertension.

Diabetes is associated with damage to many tissues, largely because hyperglycemia (and hypoglycemia, which can result from excessive or poorly timed doses of insulin) is a significant source of oxidative stress. Chronic kidney failure, retinopathy, peripheral neuropathy, peripheral vasculitis, and the development of dermal ulcers that heal slowly or not at all are among the common complications of diabetes. Because of their ability to protect against oxidative stress, particularly by the induction of HO-1 expression, compounds of the invention may be used in treatments for many complications of diabetes. As noted above (Cai et al., 2005), chronic inflammation and oxidative stress in the liver are suspected to be primary contributing factors in the development of Type 2 diabetes. Furthermore, PPARγagonists such as thiazolidinediones are capable of reducing insulin resistance and are known to be effective treatments for Type 2 diabetes.

Based on experimental results obtained, including those presented in this application, the compounds of this invention may be used for treating patients with neuroinflammation.

The effect of treatment of diabetes may be evaluated as follows. Both the biological efficacy of the treatment modality as well as the clinical efficacy are evaluated, if possible. For example, disease manifests itself by increased blood sugar, the biological efficacy of the treatment therefore can be evaluated, for example, by observation of return of the evaluated blood glucose towards normal. Measuring a clinical endpoint which can give an indication of b-cell regeneration after, for example, a six-month period of time, can give an indication of the clinical efficacy of the treatment regimen.

Based on experimental results obtained, including those presented in this application, the compounds of this invention may be used for treating patients with diabetes.

F. Rheumatoid Arthritis

Typically the first signs of rheumatoid arthritis (RA) appear in the synovial lining layer, with proliferation of synovial fibroblasts and their attachment to the articular surface at the joint margin (Lipsky, 1998). Subsequently, macrophages, T cells and other inflammatory cells are recruited into the joint, where they produce a number of mediators, including the cytokines interleukin-1 (IL-1), which contributes to the chronic sequelae leading to bone and cartilage destruction, and tumour necrosis factor (TNF-α), which plays a role in inflammation (Dinarello, 1998; Arend and Dayer, 1995; van den Berg, 2001). The concentration of IL-1 in plasma is significantly higher in patients with RA than in healthy individuals and, notably, plasma IL-1 levels correlate with RA disease activity (Eastgate et al., 1988). Moreover, synovial fluid levels of IL-1 are correlated with various radiographic and histologic features of RA (Kahle et al., 1992; Rooney et al., 1990).

In normal joints, the effects of these and other proinflammatory cytokines are balanced by a variety of anti-inflammatory cytokines and regulatory factors (Burger and Dayer, 1995). The significance of this cytokine balance is illustrated in juvenile RA patients, who have cyclical increases in fever throughout the day (Prieur et al., 1987). After each peak in fever, a factor that blocks the effects of IL-1 is found in serum and urine. This factor has been isolated, cloned and identified as IL-1 receptor antagonist (IL-1 ra), a member of the IL-1 gene family (Hannum et al., 1990). IL-1 ra, as its name indicates, is a natural receptor antagonist that competes with IL-1 for binding to type I IL-1 receptors and, as a result, blocks the effects of IL-1 (Arend et al., 1998). A 10- to 100-fold excess of IL-1 ra may be needed to block IL-1 effectively; however, synovial cells isolated from patients with RA do not appear to produce enough IL-1 ra to counteract the effects of IL-1 (Firestein et al., 1994; Fujikawa et al., 1995).

Based on experimental results obtained, including those presented in this application, the compounds of this invention may be used for treating patients with RA.

G. Psoriatic Arthritis

Psoriasis is an inflammatory and proliferative skin disorder with a prevalence of 1.5-3%. Approximately 20% of patients with psoriasis develop a characteristic form of arthritis that has several patterns (Gladman, 1992; Jones et al., 1994; Gladman et al., 1995). Some individuals present with joint symptoms first but in the majority, skin psoriasis presents first. About one-third of patients have simultaneous exacerbations of their skin and joint disease (Gladman et al., 1987) and there is a topographic relationship between nail and distal interphalangeal joint disease (Jones et al., 1994; Wright, 1956). Although the inflammatory processes which link skin, nail and joint disease remain elusive, an immune-mediated pathology is implicated.

Psoriatic arthritis (PsA) is a chronic inflammatory arthropathy characterized by the association of arthritis and psoriasis and was recognized as a clinical entity distinct from rheumatoid arthritis (RA) in 1964 (Blumberg et al., 1964). Subsequent studies have revealed that PsA shares a number of genetic, pathogenic and clinical features with other spondyloarthropathies (SpAs), a group of diseases that comprise ankylosing spondylitis, reactive arthritis and enteropathic arthritis (Wright, 1979). The notion that PsA belongs to the SpA group has recently gained further support from imaging studies demonstrating widespread enthesitis in the, including PsA but not RA (McGonagle et al., 1999; McGonagle et al., 1998). More specifically, enthesitis has been postulated to be one of the earliest events occurring in the SpAs, leading to bone remodeling and ankylosis in the spine, as well as to articular synovitis when the inflamed entheses are close to peripheral joints. However, the link between enthesitis and the clinical manifestations in PsA remains largely unclear, as PsA can present with fairly heterogeneous patterns of joint involvement with variable degrees of severity (Marsal et al., 1999; Salvarani et al., 1998). Thus, other factors must be posited to account for the multifarious features of PsA, only a few of which (such as the expression of the HLA-B27 molecule, which is strongly associated with axial disease) have been identified. As a consequence, it remains difficult to map the disease manifestations to specific pathogenic mechanisms, which means that the treatment of this condition remains largely empirical.

Family studies have suggested a genetic contribution to the development of PsA (Moll and Wright, 1973). Other chronic inflammatory forms of arthritis, such as ankylosing spondylitis and rheumatoid arthritis, are thought to have a complex genetic basis. However, the genetic component of PsA has been difficult to assess for several reasons. There is strong evidence for a genetic predisposition to psoriasis alone that may mask the genetic factors that are important for the development of PsA. Although most would accept PsA as a distinct disease entity, at times there is a phenotypic overlap with rheumatoid arthritis and ankylosing spondylitis. Also, PsA itself is not a homogeneous condition and various subgroups have been proposed.

Increased amounts of TNF-α have been reported in both psoriatic skin (Ettehadi et al., 1994) and synovial fluid (Partsch et al., 1997). Recent trials have shown a positive benefit of anti-TNF treatment in both PsA (Mease et al., 2000) and ankylosing spondylitis (Brandt et al., 2000).

Based on experimental results obtained, including those presented in this application, the compounds of this invention may be used for treating patients with psoriatic arthritis.

H. Reactive Arthritis

In reactive arthritis (ReA) the mechanism of joint damage is unclear, but it is likely that cytokines play critical roles. A more prevalent Th1 profile high levels of interferon gamma (IFN-γ) and low levels of interleukin 4 (IL-4) has been reported (Lahesmaa et al., 1992; Schlaak et al., 1992; Simon et al., 1993; Schlaak et al., 1996; Kotake et al., 1999; Ribbens et al., 2000), but several studies have shown relative predominance of IL-4 and IL-10 and relative lack of IFN-γ and tumour necrosis factor alpha (TNF-α) in the synovial membrane (Simon et al., 1994; Yin et al., 1999) and fluid (SF) (Yin et al., 1999; Yin et al., 1997) of reactive arthritis patients compared with rheumatoid arthritis (RA) patients. A lower level of TNF-α secretion in reactive arthritis than in RA patients has also been reported after ex vivo stimulation of peripheral blood mononuclear cells (PBMC) (Braun et al., 1999).

It has been argued that clearance of reactive arthritis-associated bacteria requires the production of appropriate levels of IFN-γ and TNF-α, while IL-10 acts by suppressing these responses (Autenrieth et al., 1994; Sieper and Braun, 1995). IL-10 is a regulatory cytokine that inhibits the synthesis of IL-12 and TNF-γ by activated macrophages (de Waal et al., 1991; Hart et al., 1995; Chomarat et al., 1995) and of IFN-γ by T cells (Macatonia et al., 1993).

Based on experimental results obtained, including those presented in this application, the compounds of this invention may be used for treating patients with reactive arthritis.

I. Enteropathic Arthritis

Typically enteropathic arthritis (EA) occurs in combination with inflammatory bowel diseases (IBD) such as Crohn's disease or ulcerative colitis. It also can affect the spine and sacroiliac joints. Enteropathic arthritis involves the peripheral joints, usually in the lower extremities such as the knees or ankles. It commonly involves only a few or a limited number of joints and may closely follow the bowel condition. This occurs in approximately 11% of patients with ulcerative colitis and 21% of those with Crohn's disease. The synovitis is generally self-limited and non-deforming.

Enteropathic arthropathies comprise a collection of rheumatologic conditions that share a link to GI pathology. These conditions include reactive (i.e., infection-related) arthritis due to bacteria (e.g., Shigella, Salmonella, Campylobacter, Yersinia species, Clostridium difficile), parasites (e.g., Stronzyloides stercoralis, Taenia saginata, Giardia lamblia, Ascaris lumbricoides, Cryptosporidium species), and spondyloarthropathies associated with inflammatory bowel disease (IBD). Other conditions and disorders include intestinal bypass (jejunoileal), arthritis, celiac disease, Whipple disease, and collagenous colitis.

Based on experimental results obtained, including those presented in this application, the compounds of this invention may be used for treating patients with enteropathic arthritis.

J. Juvenile Rheumatoid Arthritis

Juvenile rheumatoid arthritis (JRA), a term for the most prevalent form of arthritis in children, is applied to a family of illnesses characterized by chronic inflammation and hypertrophy of the synovial membranes. The term overlaps, but is not completely synonymous, with the family of illnesses referred to as juvenile chronic arthritis and/or juvenile idiopathic arthritis in Europe.

Both innate and adaptive immune systems use multiple cell types, a vast array of cell surface and secreted proteins, and interconnected networks of positive and negative feedback (Lo et al., 1999). Furthermore, while separable in thought, the innate and adaptive wings of the immune system are functionally intersected (Fearon and Locksley, 1996), and pathologic events occurring at these intersecting points are likely to be highly relevant to our understanding of pathogenesis of adult and childhood forms of chronic arthritis (Warrington, et al., 2001).

Polyarticular JRA is a distinct clinical subtype characterized by inflammation and synovial proliferation in multiple joints (four or more), including the small joints of the hands (Jarvis, 2002). This subtype of JRA may be severe, because of both its multiple joint involvement and its capacity to progress rapidly over time. Although clinically distinct, polyarticular JRA is not homogeneous, and patients vary in disease manifestations, age of onset, prognosis, and therapeutic response. These differences very likely reflect a spectrum of variation in the nature of the immune and inflammatory attack that can occur in this disease (Jarvis, 1998).

Based on experimental results obtained, including those presented in this application, the compounds of this invention may be used for treating patients with JRA.

K. Early Inflammatory Arthritis

The clinical presentation of different inflammatory arthropathies is similar early in the course of disease. As a result, it is often difficult to distinguish patients who are at risk of developing the severe and persistent synovitis that leads to erosive joint damage from those whose arthritis is more self-limited.

Recent efforts to identify predictors of poor outcome in early inflammatory arthritis have identified the presence of RA specific autoantibodies, in particular antibodies towards citrullinated peptides, to be associated with erosive and persistent disease in early inflammatory arthritis cohorts. On the basis of this, a cyclical citrullinated peptide (CCP) has been developed to assist in the identification of anti-CCP antibodies in patient sera. Using this approach, the presence of anti-CCP antibodies has been shown to be specific and sensitive for RA, can distinguish RA from other arthropathies, and can potentially predict persistent, erosive synovitis before these outcomes become clinically manifest (Schellekens et al., 2000). Importantly, anti-CCP antibodies are often detectable in sera many years prior to clinical symptoms suggesting that they may be reflective of subclinical immune events (Nielen et al., 2004; Rantapaa-Dahlqvist et al., 2003).

The clinical presentation of different inflammatory arthropathies is similar early in the course of disease. As a result, it is often difficult to distinguish patients who are at risk of developing the severe and persistent synovitis that leads to erosive joint damage from those whose arthritis is more self-limited. Such distinction is critical in order to target therapy appropriately, treating aggressively those with erosive disease and avoiding unnecessary toxicity in patients with more self-limited disease. Current clinical criteria for diagnosing erosive arthropathies such as rheumatoid arthritis (RA) are less effective in early disease and traditional markers of disease activity such as joint counts and acute phase response do not adequately identify patients likely to have poor outcomes (Harrison et al., 1998). Parameters reflective of the pathologic events occurring in the synovium are most likely to be of significant prognostic value.

Recent efforts to identify predictors of poor outcome in early inflammatory arthritis have identified the presence of RA specific autoantibodies, in particular antibodies towards citrullinated peptides, to be associated with erosive and persistent disease in early inflammatory arthritis cohorts. On the basis of this, a cyclical citrullinated peptide (CCP) has been developed to assist in the identification of anti-CCP antibodies in patient sera. Using this approach, the presence of anti-CCP antibodies has been shown to be specific and sensitive for RA, can distinguish RA from other arthropathies, and can potentially predict persistent, erosive synovitis before these outcomes become clinically manifest. Importantly, anti-CCP antibodies are often detectable in sera many years prior to clinical symptoms suggesting that they may be reflective of subclinical immune events (Nielen et al., 2004; Rantapaa-Dahlqvist et al., 2003).

Based on experimental results obtained, including those presented in this application, the compounds of this invention may be used for treating patients with early inflammatory arthritis.

L. Ankylosing Spondylitis

AS is a disease subset within a broader disease classification of spondyloarthropathy. Patients affected with the various subsets of spondyloarthropathy have disease etiologies that are often very different, ranging from bacterial infections to inheritance. Yet, in all subgroups, the end result of the disease process is axial arthritis. Despite the early clinically differences seen in the various patient populations, many of them end up nearly identical after a disease course of ten-to-twenty years. Recent studies suggest the mean time to clinical diagnosis of ankylosing spondylitis from disease onset of disease is 7.5 years (Khan, 1998). These same studies suggest that the spondyloarthropathies may have prevalence close to that of rheumatoid arthritis (Feldtkeller et al., 2003; Doran et al., 2003).

AS is a chronic systemic inflammatory rheumatic disorder of the axial skeleton with or without extraskeletal manifestations. Sacroiliac joints and the spine are primarily affected, but hip and shoulder joints, and less commonly peripheral joints or certain extra-articular structures such as the eye, vasculature, nervous system, and gastrointestinal system may also be involved. Its etiology is not yet fully understood (Wordsworth, 1995; Calin and Taurog, 1998). It is strongly associated with the major histocompatibility class I (MHC I) HLA-B27 allele (Calin and Taurog, 1998). AS affects individuals in the prime of their life and is feared because of its potential to cause chronic pain and irreversible damage of tendons, ligaments, joints, and bones (Brewerton et al., 1973a; Brewerton et al., 1973b; Schlosstein et al., 1973). AS may occur alone or in association with another form of spondyloarthropathy such as reactive arthritis, psoriasis, psoriatic arthritis, enthesitis, ulcerative colitis, irritable bowel disease, or Crohn's disease, in which case it is classified as secondary AS.

Typically, the affected sites include the discovertebral, apophyseal, costovertebral, and costotransverse joints of the spine, and the paravertebral ligamentous structures. Inflammation of the entheses, which are sites of musculotendinous and ligamentous attachment to bones, is also prominent in this disease (Calin and Taurog, 1998). The site of enthesitis is known to be infiltrated by plasma cells, lymphocytes, and polymorphonuclear cells. The inflammatory process frequently results in gradual fibrous and bony ankylosis, (Ball, 1971; Khan, 1990).

Delayed diagnosis is common because symptoms are often attributed to more common back problems. A dramatic loss of flexibility in the lumbar spine is an early sign of AS. Other common symptoms include chronic pain and stiffness in the lower back which usually starts where the lower spine is joined to the pelvis, or hip. Although most symptoms begin in the lumbar and sacroiliac areas, they may involve the neck and upper back as well. Arthritis may also occur in the shoulder, hips and feet. Some patients have eye inflammation, and more severe cases must be observed for heart valve involvement.

The most frequent presentation is back pain, but disease can begin atypically in peripheral joints, especially in children and women, and rarely with acute iritis (anterior uveitis). Additional early symptoms and signs are diminished chest expansion from diffuse costovertebral involvement, low-grade fever, fatigue, anorexia, weight loss, and anemia. Recurrent back pain—often nocturnal and of varying intensity—is an eventual complaint, as is morning stiffness typically relieved by activity. A flexed or bent-over posture eases back pain and paraspinal muscle spasm; thus, some degree of kyphosis is common in untreated patients.

Systemic manifestations occur in ⅓ of patients. Recurrent, usually self-limited, acute iritis (anterior uveitis) rarely is protracted and severe enough to impair vision. Neurologic signs can occasionally result from compression radiculitis or sciatica, vertebral fracture or subluxation, and cauda equina syndrome (which consists of impotence, nocturnal urinary incontinence, diminished bladder and rectal sensation, and absence of ankle jerks). Cardiovascular manifestations can include aortic insufficiency, angina, pericarditis, and ECG conduction abnormalities. A rare pulmonary finding is upper lobe fibrosis, occasionally with cavitation that may be mistaken for TB and can be complicated by infection with Aspergillus.

AS is characterized by mild or moderate flares of active spondylitis alternating with periods of almost or totally inactive inflammation. Proper treatment in most patients results in minimal or no disability and in full, productive lives despite back stiffness. Occasionally, the course is severe and progressive, resulting in pronounced incapacitating deformities. The prognosis is bleak for patients with refractory iritis and for the rare patient with secondary amyloidosis.

Based on experimental results obtained, including those presented in this application, the compounds of this invention may be used for treating patients with ankylosing spondylitis.

M. Ulcerative Colitis

Ulcerative colitis is a disease that causes inflammation and sores, called ulcers, in the lining of the large intestine. The inflammation usually occurs in the rectum and lower part of the colon, but it may affect the entire colon. Ulcerative colitis rarely affects the small intestine except for the end section, called the terminal ileum. Ulcerative colitis may also be called colitis or proctitis. The inflammation makes the colon empty frequently, causing diarrhea. Ulcers form in places where the inflammation has killed the cells lining the colon; the ulcers bleed and produce pus.

Ulcerative colitis is an inflammatory bowel disease (IBD), the general name for diseases that cause inflammation in the small intestine and colon. Ulcerative colitis can be difficult to diagnose because its symptoms are similar to other intestinal disorders and to another type of IBD, Crohn's disease. Crohn's disease differs from ulcerative colitis because it causes inflammation deeper within the intestinal wall. Also, Crohn's disease usually occurs in the small intestine, although it can also occur in the mouth, esophagus, stomach, duodenum, large intestine, appendix, and anus.

Ulcerative colitis may occur in people of any age, but most often it starts between ages 15 and 30, or less frequently between ages 50 and 70. Children and adolescents sometimes develop the disease. Ulcerative colitis affects men and women equally and appears to run in some families. Theories about what causes ulcerative colitis abound, but none have been proven. The most popular theory is that the body's immune system reacts to a virus or a bacterium by causing ongoing inflammation in the intestinal wall. People with ulcerative colitis have abnormalities of the immune system, but doctors do not know whether these abnormalities are a cause or a result of the disease. Ulcerative colitis is not caused by emotional distress or sensitivity to certain foods or food products, but these factors may trigger symptoms in some people.

The most common symptoms of ulcerative colitis are abdominal pain and bloody diarrhea. Patients also may experience fatigue, weight loss, loss of appetite, rectal bleeding, and loss of body fluids and nutrients. About half of patients have mild symptoms. Others suffer frequent fever, bloody diarrhea, nausea, and severe abdominal cramps. Ulcerative colitis may also cause problems such as arthritis, inflammation of the eye, liver disease (hepatitis, cirrhosis, and primary sclerosing cholangitis), osteoporosis, skin rashes, and anemia. No one knows for sure why problems occur outside the colon. Scientists think these complications may occur when the immune system triggers inflammation in other parts of the body. Some of these problems go away when the colitis is treated.

A thorough physical exam and a series of tests may be required to diagnose ulcerative colitis. Blood tests may be done to check for anemia, which could indicate bleeding in the colon or rectum. Blood tests may also uncover a high white blood cell count, which is a sign of inflammation somewhere in the body. By testing a stool sample, the doctor can detect bleeding or infection in the colon or rectum. The doctor may do a colonoscopy or sigmoidoscopy. For either test, the doctor inserts an endoscope—a long, flexible, lighted tube connected to a computer and TV monitor—into the anus to see the inside of the colon and rectum. The doctor will be able to see any inflammation, bleeding, or ulcers on the colon wall. During the exam, the doctor may do a biopsy, which involves taking a sample of tissue from the lining of the colon to view with a microscope. A barium enema x ray of the colon may also be required. This procedure involves filling the colon with barium, a chalky white solution. The barium shows up white on x-ray film, allowing the doctor a clear view of the colon, including any ulcers or other abnormalities that might be there.

Treatment for ulcerative colitis depends on the seriousness of the disease. Most people are treated with medication. In severe cases, a patient may need surgery to remove the diseased colon. Surgery is the only cure for ulcerative colitis. Some people whose symptoms are triggered by certain foods are able to control the symptoms by avoiding foods that upset their intestines, like highly seasoned foods, raw fruits and vegetables, or milk sugar (lactose). Each person may experience ulcerative colitis differently, so treatment is adjusted for each individual. Emotional and psychological support is important. Some people have remissions—periods when the symptoms go away—that last for months or even years. However, most patients' symptoms eventually return. This changing pattern of the disease means one cannot always tell when a treatment has helped. Some people with ulcerative colitis may need medical care for some time, with regular doctor visits to monitor the condition.

Based on experimental results obtained, including those presented in this application, the compounds of this invention may be used for treating patients with ankylosing spondylitis.

N. Crohn's Disease

Another disorder for which immunosuppression has been tried is Crohn's disease. Crohn's disease symptoms include intestinal inflammation and the development of intestinal stenosis and fistulas; neuropathy often accompanies these symptoms. Anti-inflammatory drugs, such as 5-aminosalicylates (e.g., mesalamine) or corticosteroids, are typically prescribed, but are not always effective (reviewed in Botoman et al., 1998). Immunosuppression with cyclosporine is sometimes beneficial for patients resistant to or intolerant of corticosteroids (Brynskov et al., 1989).

Efforts to develop diagnostic and treatment tools against Crohn's disease have focused on the central role of cytokines (Schreiber, 1998; van Hogezand and Verspaget, 1998). Cytokines are small secreted proteins or factors (5 to 20 kD) that have specific effects on cell-to-cell interactions, intercellular communication, or the behavior of other cells. Cytokines are produced by lymphocytes, especially T_{H}1 and T_{H}2 lymphocytes, monocytes, intestinal macrophages, granulocytes, epithelial cells, and fibroblasts (reviewed in Rogler and. Andus, 1998; Galley and Webster, 1996). Some cytokines are pro-inflammatory (e.g., TNF-α, IL-1(α and β), IL-6, IL-8, IL-12, or leukemia inhibitory factor [LIF]); others are anti-inflammatory (e.g., IL-1 receptor antagonist, IL-4, IL-10, IL-11, and TGF-β). However, there may be overlap and functional redundancy in their effects under certain inflammatory conditions.

In active cases of Crohn's disease, elevated concentrations of TNF-α and IL-6 are secreted into the blood circulation, and TNF-α, IL-1, IL-6, and IL-8 are produced in excess locally by mucosal cells (id.; Funakoshi et al., 1998). These cytokines can have far-ranging effects on physiological systems including bone development, hematopoiesis, and liver, thyroid, and neuropsychiatric function. Also, an imbalance of the IL-1β/IL-1 ra ratio, in favor of pro-inflammatory IL-1β, has been observed in patients with Crohn's disease (Rogler and Andus, 1998; Saiki et al., 1998; Dionne et al., 1998; but see Kuboyama, 1998). One study suggested that cytokine profiles in stool samples could be a useful diagnostic tool for Crohn's disease (Saiki et al., 1998).

Treatments that have been proposed for Crohn's disease include the use of various cytokine antagonists (e.g., IL-1 ra), inhibitors (e.g., of IL-1β converting enzyme and antioxidants) and anti-cytokine antibodies (Rogler and Andus, 1998; van Hogezand and Verspaget, 1998; Reimund et al., 1998; Lugering et al., 1998; McAlindon et al., 1998). In particular, monoclonal antibodies against TNF-α have been tried with some success in the treatment of Crohn's disease (Targan et al., 1997; Stack et al., 1997; van Dullemen et al., 1995). These compounds may be used in combination therapy with compounds of the present invention.

Another approach to the treatment of Crohn's disease has focused on at least partially eradicating the bacterial community that may be triggering the inflammatory response and replacing it with a non-pathogenic community. For example, U.S. Pat. No. 5,599,795 discloses a method for the prevention and treatment of Crohn's disease in human patients. Their method was directed to sterilizing the intestinal tract with at least one antibiotic and at least one anti-fungal agent to kill off the existing flora and replacing them with different, select, well-characterized bacteria taken from normal humans. Borody taught a method of treating Crohn's disease by at least partial removal of the existing intestinal microflora by lavage and replacement with a new bacterial community introduced by fecal inoculum from a disease-screened human donor or by a composition comprising Bacteroides and Escherichia coli species. (U.S. Pat. No. 5,443,826).

Based on experimental results obtained, including those presented in this application, the compounds of this invention may be used for treating patients with Crohn's disease.

O. Systemic Lupus Erythematosus

There has also been no known cause for autoimmune diseases such as systemic lupus erythematosus. Systemic lupus erythematosus (SLE) is an autoimmune rheumatic disease characterized by deposition in tissues of autoantibodies and immune complexes leading to tissue injury (Kotzin, 1996). In contrast to autoimmune diseases such as MS and type 1 diabetes mellitus, SLE potentially involves multiple organ systems directly, and its clinical manifestations are diverse and variable (reviewed by Kotzin and O'Dell, 1995). For example, some patients may demonstrate primarily skin rash and joint pain, show spontaneous remissions, and require little medication. At the other end of the spectrum are patients who demonstrate severe and progressive kidney involvement that requires therapy with high doses of steroids and cytotoxic drugs such as cyclophosphamide (Kotzin, 1996).

The serological hallmark of SLE, and the primary diagnostic test available, is elevated serum levels of IgG antibodies to constituents of the cell nucleus, such as double-stranded DNA (dsDNA), single-stranded DNA (ss-DNA), and chromatin. Among these autoantibodies, IgG anti-dsDNA antibodies play a major role in the development of lupus glomerulonephritis (G N) (Hahn and Tsao, 1993; Ohnishi et al., 1994). Glomerulonephritis is a serious condition in which the capillary walls of the kidney's blood purifying glomeruli become thickened by accretions on the epithelial side of glomerular basement membranes. The disease is often chronic and progressive and may lead to eventual renal failure.

Based on experimental results obtained, including those presented in this application, the compounds of this invention may be used for treating patients with SLE.

P. Irritable Bowel Syndrome

Irritable bowel syndrome (IBS) is a functional disorder characterized by abdominal pain and altered bowel habits. This syndrome may begin in young adulthood and can be associated with significant disability. This syndrome is not a homogeneous disorder. Rather, subtypes of IBS have been described on the basis of the predominant symptom—diarrhea, constipation, or pain. In the absence of “alarm” symptoms, such as fever, weight loss, and gastrointestinal bleeding, a limited workup is needed. Once a diagnosis of IBS is made, an integrated treatment approach can effectively reduce the severity of symptoms. IBS is a common disorder, although its prevalence rates have varied. In general, IBS affects about 15% of US adults and occurs about three times more often in women than in men (Jailwala et al., 2000).

IBS accounts for between 2.4 million and 3.5 million visits to physicians each year. It not only is the most common condition seen by gastroenterologists but also is one of the most common gastrointestinal conditions seen by primary care physicians (Everhart et al., 1991; Sandler, 1990).

IBS is also a costly disorder. Compared with persons who do not have bowel symptoms, persons with IBS miss three times as many workdays and are more likely to report being too sick to work (Drossman et al., 1993; Drossman et al., 1997). Moreover, those with IBS incur hundreds of dollars more in medical charges than persons without bowel disorders (Talley et al., 1995).

No specific abnormality accounts for the exacerbations and remissions of abdominal pain and altered bowel habits experienced by patients with IBS. The evolving theory of IBS suggests dysregulation at multiple levels of the brain-gut axis. Dysmotility, visceral hypersensitivity, abnormal modulation of the central nervous system (CNS), and infection have all been implicated. In addition, psychosocial factors play an important modifying role. Abnormal intestinal motility has long been considered a factor in the pathogenesis of IBS. Transit time through the small intestine after a meal has been shown to be shorter in patients with diarrhea-predominant IBS than in patients who have the constipation-predominant or pain-predominant subtype (Cann et al., 1983).

In studies of the small intestine during fasting, the presence of both discrete, clustered contractions and prolonged, propagated contractions has been reported in patients with IBS (Kellow and Phillips, 1987). They also experience pain with irregular contractions more often than healthy persons (Kellow and Phillips, 1987; Horwitz and Fisher, 2001)

These motility findings do not account for the entire symptom complex in patients with IBS; in fact, most of these patients do not have demonstrable abnormalities (Rothstein, 2000). Patients with IBS have increased sensitivity to visceral pain. Studies involving balloon distention of the rectosigmoid colon have shown that patients with IBS experience pain and bloating at pressures and volumes much lower than control subjects (Whitehead et al., 1990). These patients maintain normal perception of somatic stimuli.

Multiple theories have been proposed to explain this phenomenon. For example, receptors in the viscera may have increased sensitivity in response to distention or intraluminal contents. Neurons in the dorsal horn of the spinal cord may have increased excitability. In addition, alteration in CNS processing of sensations may be involved (Drossman et al., 1997). Functional magnetic resonance imaging studies have recently shown that compared with control subjects, patients with IBS have increased activation of the anterior cingulate cortex, an important pain center, in response to a painful rectal stimulus (Mertz et al., 2000).

Increasingly, evidence suggests a relationship between infectious enteritis and subsequent development of IBS. Inflammatory cytokines may play a role. In a survey of patients with a history of confirmed bacterial gastroenteritis (Neal et al., 1997), 25% reported persistent alteration of bowel habits. Persistence of symptoms may be due to psychologic stress at the time of acute infection (Gwee et al., 1999).

Recent data suggest that bacterial overgrowth in the small intestine may have a role in IBS symptoms. In one study (Pimentel et al., 2000), 157 (78%) of 202 IBS patients referred for hydrogen breath testing had test findings that were positive for bacterial overgrowth. Of the 47 subjects who had follow-up testing, 25 (53%) reported improvement in symptoms (i.e., abdominal pain and diarrhea) with antibiotic treatment.

IBS may present with a range of symptoms. However, abdominal pain and altered bowel habits remain the primary features. Abdominal discomfort is often described as crampy in nature and located in the left lower quadrant, although the severity and location can differ greatly. Patients may report diarrhea, constipation, or alternating episodes of diarrhea and constipation. Diarrheal symptoms are typically described as small-volume, loose stools, and stool is sometimes accompanied by mucus discharge. Patients also may report bloating, fecal urgency, incomplete evacuation, and abdominal distention. Upper gastrointestinal symptoms, such as gastroesophageal reflux, dyspepsia, or nausea, may also be present (Lynn and Friedman, 1993).

Persistence of symptoms is not an indication for further testing; it is a characteristic of IBS and is itself an expected symptom of the syndrome. More extensive diagnostic evaluation is indicated in patients whose symptoms are worsening or changing. Indications for further testing also include presence of alarm symptoms, onset of symptoms after age 50, and a family history of colon cancer. Tests may include colonoscopy, computed tomography of the abdomen and pelvis, and barium studies of the small or large intestine.

Based on experimental results obtained, including those presented in this application, the compounds of this invention may be used for treating patients with IBS.

Q. Sjögren's syndrome

Primary Sjögren's syndrome (SS) is a chronic, slowly progressive, systemic autoimmune disease, which affects predominantly middle-aged women (female-to-male ratio 9:1), although it can be seen in all ages including childhood (Jonsson et al., 2002). It is characterized by lymphocytic infiltration and destruction of the exocrine glands, which are infiltrated by mononuclear cells including CD4+, CD8+ lymphocytes and B-cells (Jonsson et al., 2002). In addition, extraglandular (systemic) manifestations are seen in one-third of patients (Jonsson et al., 2001).

The glandular lymphocytic infiltration is a progressive feature (Jonsson et al., 1993), which, when extensive, may replace large portions of the organs. Interestingly, the glandular infiltrates in some patients closely resemble ectopic lymphoid microstructures in the salivary glands (denoted as ectopic germinal centers) (Salomonsson et al., 2002; Xanthou et al., 2001). In SS, ectopic GCs are defined as T and B cell aggregates of proliferating cells with a network of follicular dendritic cells and activated endothelial cells. These GC-like structures formed within the target tissue also portray functional properties with production of autoantibodies (anti-Ro/SSA and anti-La/SSB) (Salomonsson and Jonsson, 2003).

In other systemic autoimmune diseases, such as RA, factors critical for ectopic GCs have been identified. Rheumatoid synovial tissues with GCs were shown to produce chemokines CXCL13, CCL21 and lymphotoxin (LT)-β (detected on follicular center and mantle zone B cells). Multivariate regression analysis of these analytes identified CXCL13 and LT-β as the solitary cytokines predicting GCs in rheumatoid synovitis (Weyand and Goronzy, 2003). Recently CXCL13 and CXCR5 in salivary glands has been shown to play an essential role in the inflammatory process by recruiting B and T cells, therefore contributing to lymphoid neogenesis and ectopic GC formation in SS (Salomonsson et al., 2002).

Based on experimental results obtained, including those presented in this application, the compounds of this invention may be used for treating patients with SS.

R. Psoriasis

Psoriasis is a chronic skin disease of scaling and inflammation that affects 2 to 2.6 percent of the United States population, or between 5.8 and 7.5 million people. Although the disease occurs in all age groups, it primarily affects adults. It appears about equally in males and females. Psoriasis occurs when skin cells quickly rise from their origin below the surface of the skin and pile up on the surface before they have a chance to mature. Usually this movement (also called turnover) takes about a month, but in psoriasis it may occur in only a few days. In its typical form, psoriasis results in patches of thick, red (inflamed) skin covered with silvery scales. These patches, which are sometimes referred to as plaques, usually itch or feel sore. They most often occur on the elbows, knees, other parts of the legs, scalp, lower back, face, palms, and soles of the feet, but they can occur on skin anywhere on the body. The disease may also affect the fingernails, the toenails, and the soft tissues of the genitals and inside the mouth. While it is not unusual for the skin around affected joints to crack, approximately 1 million people with psoriasis experience joint inflammation that produces symptoms of arthritis. This condition is called psoriatic arthritis.

Psoriasis is a skin disorder driven by the immune system, especially involving a type of white blood cell called a T cell. Normally, T cells help protect the body against infection and disease. In the case of psoriasis, T cells are put into action by mistake and become so active that they trigger other immune responses, which lead to inflammation and to rapid turnover of skin cells. In about one-third of the cases, there is a family history of psoriasis. Researchers have studied a large number of families affected by psoriasis and identified genes linked to the disease. People with psoriasis may notice that there are times when their skin worsens, then improves. Conditions that may cause flareups include infections, stress, and changes in climate that dry the skin. Also, certain medicines, including lithium and betablockers, which are prescribed for high blood pressure, may trigger an outbreak or worsen the disease.

Based on experimental results obtained, including those presented in this application, the compounds of this invention may be used for treating patients with psoriasis.

### VIII. Pharmaceutical Formulations and Routes of Administration

The compounds of the present invention may be administered by a variety of methods, e.g., orally or by injection (e.g., subcutaneous, intravenous, intraperitoneal, etc.). Depending on the route of administration, the active compounds may be coated in a material to protect the compound from the action of acids and other natural conditions which may inactivate the compound. They may also be administered by continuous perfusion/infusion of a disease or wound site.

To administer the therapeutic compound by other than parenteral administration, it may be necessary to coat the compound with, or co-administer the compound with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., 1984).

The therapeutic compound may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

The therapeutic compound may also be administered topically to the skin, eye, or mucosa. Alternatively, if local delivery to the lungs is desired the therapeutic compound may be administered by inhalation in a dry-powder or aerosol formulation.

Active compounds are administered at a therapeutically effective dosage sufficient to treat a condition associated with a condition in a patient. A “therapeutically effective amount” preferably reduces the amount of symptoms of the condition in the infected patient by at least about 20%, more preferably by at least about 40%, even more preferably by at least about 60%, and still more preferably by at least about 80% relative to untreated subjects. For example, the efficacy of a compound can be evaluated in an animal model system that may be predictive of efficacy in treating the disease in humans, such as the model systems shown in the examples and drawings.

The actual dosage amount of a compound of the present invention or composition comprising a compound of the present invention administered to a subject may be determined by physical and physiological factors such as age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

An effective amount typically will vary from about 0.001 mg/kg to about 1000 mg/kg, from about 0.01 mg/kg to about 750 mg/kg, from about 100 mg/kg to about 500 mg/kg, from about 1.0 mg/kg to about 250 mg/kg, from about 10.0 mg/kg to about 150 mg/kg in one or more dose administrations daily, for one or several days (depending of course of the mode of administration and the factors discussed above). Other suitable dose ranges include 1 mg to 10000 mg per day, 100 mg to 10000 mg per day, 500 mg to 10000 mg per day, and 500 mg to 1000 mg per day. In some particular embodiments, the amount is less than 10,000 mg per day with a range of 750 mg to 9000 mg per day.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day. For example, regarding treatment of diabetic patients, the unit dosage may be an amount that reduces blood glucose by at least 40% as compared to an untreated subject. In another embodiment, the unit dosage is an amount that reduces blood glucose to a level that is ±10% of the blood glucose level of a non-diabetic subject.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a pharmaceutical composition of the present invention may comprise, for example, at least about 0.1% of a compound of the present invention. In other embodiments, the compound of the present invention may comprise between about 2% to about 75% of the weight of the unit, or between about 25% to about 60%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may taken orally and that the timing of which is or is not dependent upon food intake. Thus, for example, the agent can be taken every morning and/or every evening, regardless of when the subject has eaten or will eat.

### IX. Combination Therapy

In addition to being used as a monotherapy, the compounds of the present invention may also find use in combination therapies. Effective combination therapy may be achieved with a single composition or pharmacological formulation that includes both agents, or with two distinct compositions or formulations, at the same time, wherein one composition includes the oleanic acid derivative according to the methods of this invention, and the other includes the second agent(s). Alternatively, the therapy may precede or follow the other agent treatment by intervals ranging from minutes to months.

Various combinations may be employed, such as when a compound of the present invention is “A” and “B” represents a secondary agent, non-limiting examples of which are described below:

A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A

Administration of the compounds of the present invention to a patient will follow general protocols for the administration of pharmaceuticals, taking into account the toxicity, if any, of the drug. It is expected that the treatment cycles would be repeated as necessary.

Beta interferons may be suitable secondary agents. These are medications derived from human cytokines which help regulate the immune system. They include interferon β-1 b and interferon β-1 a. Betaseron has been approved by the FDA for relapsing forms of secondary progressive MS. Furthermore, the FDA has approved the use of several β-interferons as treatments for people who have experienced a single attack that suggests multiple sclerosis, and who may be at risk of future attacks and developing definite MS. For example, risk of MS may be suggested when an MRI scan of the brain shows lesions that predict a high risk of conversion to definite MS.

Glatiramer acetate is a further example of a secondary agent that may be used in a combination treatment. Glatiramer is presently used to treat relapsing remitting MS. It is made of four amino acids that are found in myelin. This drug is reported to stimulate T cells in the body's immune system to change from harmful, pro-inflammatory agents to beneficial, anti-inflammatory agents that work to reduce inflammation at lesion sites.

Another potential secondary agent is mitoxantrone, a chemotherapy drug used for many cancers. This drug is also FDA-approved for treatment of aggressive forms of relapsing remitting MS, as well as certain forms of progressive MS. It is given intravenously, typically every three months. This medication is effective, but is limited by cardiac toxicity. Novantrone has been approved by the FDA for secondary progressive, progressive-relapsing, and worsening relapsing-remitting MS.

Another potential secondary agent is natalizumab. In general, natalizumab works by blocking the attachment of immune cells to brain blood vessels, which is a necessary step for immune cells to cross into the brain, thus reducing the immune cells' inflammatory action on brain neurons. Natalizumab has been shown to significantly reduce the frequency of attacks in people with relapsing MS.

In the case of relapsing remitting MS, patients may be given intravenous corticosteroids, such as methylprednisolone, as a secondary agent, to end the attack sooner and leave fewer lasting deficits.

Other common drugs for MS that may be used in combination with the oleanic acid derivatives include immunosuppressive drugs such as azathioprine, cladribine and cyclophosphamide.

It is contemplated that other anti-inflammatory agents may be used in conjunction with the treatments of the current invention. Other COX inhibitors may be used, including arylcarboxylic acids (salicylic acid, acetylsalicylic acid, diflunisal, choline magnesium trisalicylate, salicylate, benorylate, flufenamic acid, mefenamic acid, meclofenamic acid and triflumic acid), arylalkanoic acids (diclofenac, fenclofenac, alclofenac, fentiazac, ibuprofen, flurbiprofen, ketoprofen, naproxen, fenoprofen, fenbufen, suprofen, indoprofen, tiaprofenic acid, benoxaprofen, pirprofen, tolmetin, zomepirac, clopinac, indomethacin and sulindac) and enolic acids (phenylbutazone, oxyphenbutazone, azapropazone, feprazone, piroxicam, and isoxicam. See also U.S. Pat. No. 6,025,395, which is incorporated herein by reference.

Histamine H2 receptor blocking agents may also be used in conjunction with the compounds of the current invention, including cimetidine, ranitidine, famotidine and nizatidine.

Treatment with acetylcholinesterase inhibitors such as tacrine, donepizil, metrifonate and rivastigmine for the treatment of Alzheimer's and other disease in conjunction with the compounds of the present invention is contemplated. Other acetylcholinesterase inhibitors may be developed which may be used once approved include rivastigmine and metrifonate. Acetylcholinesterase inhibitors increase the amount of neurotransmitter acetylcholine at the nerve terminal by decreasing its breakdown by the enzyme cholinesterase.

MAO-B inhibitors such as selegilene may be used in conjunction with the compounds of the current invention. Selegilene is used for Parkinson's disease and irreversibly inhibits monoamine oxidase type B (MAO-B). Monoamine oxidase is an enzyme that inactivates the monoamine neurotransmitters norepinephrine, serotonin and dopamine.

Dietary and nutritional supplements with reported benefits for treatment or prevention of Parkinson's, Alzheimer's, multiple sclerosis, amyotrophic lateral sclerosis, rheumatoid arthritis, inflammatory bowel disease, and all other diseases whose pathogenesis is believed to involve excessive production of either nitric oxide (NO) or prostaglandins, such as acetyl-L-carnitine, octacosanol, evening primrose oil, vitamin B6, tyrosine, phenylalanine, vitamin C, L-dopa, or a combination of several antioxidants may be used in conjunction with the compounds of the current invention.

For the treatment or prevention of cancer, compounds of the invention may be combined with one or more of the following: radiation, chemotherapy agents (e.g., cytotoxic agents such as anthracyclines, vincristine, vinblastin, microtubule-targeting agents such as paclitaxel and docetaxel, 5-FU and related agents, cisplatin and other platinum-containing compounds, irinotecan and topotecan, gemcitabine, temozolomide, etc.), targeted therapies (e.g., imatinib, bortezomib, bevacizumab, rituximab), or vaccine therapies designed to promote an enhanced immune response targeting cancer cells.

For the treatment or prevention of autoimmune disease, compounds of the invention may be combined with one or more of the following: corticosteroids, methotrexate, anti-TNF antibodies, other TNF-targeting protein therapies, and NSAIDs. For the treatment of prevention of cardiovascular diseases, compounds of the invention may be combined with antithrombotic therapies, anticholesterol therapies such as statins (e.g., atorvastatin), and surgical interventions such as stenting or coronary artery bypass grafting. For the treatment of osteoporosis, compounds of the invention may be combined with antiresorptive agents such as bisphosphonates or anabolic therapies such as teriparatide or parathyroid hormone. For the treatment of neuropsychiatric conditions, compounds of the invention may be combined with antidepressants (e.g., imipramine or SSRIs such as fluoxetine), antipsychotic agents (e.g., olanzapine, sertindole, risperidone), mood stabilizers (e.g., lithium, valproate semisodium), or other standard agents such as anxiolytic agents. For the treatment of neurological disorders, compounds of the invention may be combined with anticonvulsant agents (e.g., valproate semisodium, gabapentin, phenyloin, carbamazepine, and topiramate), antithrombotic agents (e.g., tissue plasminogen activator), or analgesics (e.g., opioids, sodium channel blockers, and other antinociceptive agents).

### X. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Chemicals. Triterpenoids were synthesized as previously described in Honda et al. (2002), Honda et al. (1998), and Honda et al. (2000b). The various amide derivatives were synthesized by the condensation of CDDO acid chloride with the respective amine hydrochlorides (or free amines) using variations based of methods of Honda et al. (2002). The synthesis of CDDO-MA is discussed in Honda et al. (2002), which is incorporated herein by reference. The syntheses of CDDO-EA and CDDO-TFEA are presented in Yates et al. (2007), which is incorporated herein by reference, and shown in the Scheme 1 above.

### Example 2

### Blood Brain Barrier Penetration Results

The ability of synthetic triterpenoids (TPs) to penetrate the brain of mammals varies according to their structure. As shown in FIG. 1, CDDO-Me (TP-155) is detectable, using MS analysis, in the brains of mice fed very low levels of the compound over a week.

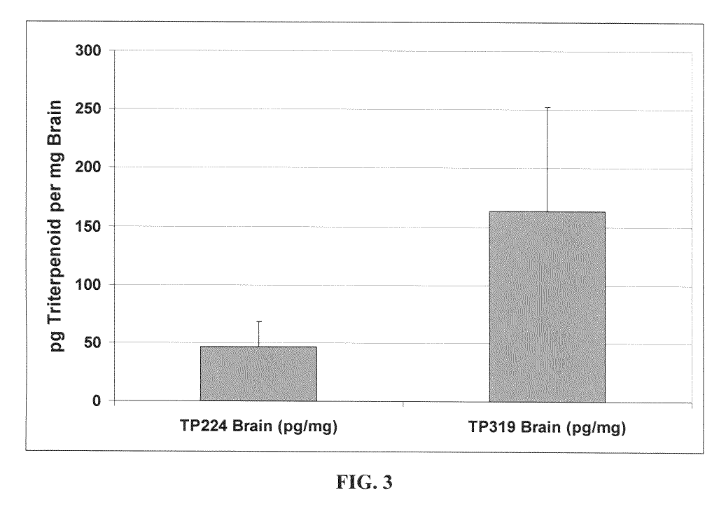

FIG. 2 shows the results of three experiments directed toward the ability of CDDO Methyl Amide (TP-224) to penetrate into the brains of mice that received TP-224 orally. In experiment 1 (Expt 1) three mice were each fed an 800 mg/kg diet of CDDO Methyl Amide (TP-224) for two days. In experiment 2 (Expt 2) three mice were each fed an 800 mg/kg diet of CDDO Methyl Amide (TP-224) for four days. In experiment 3 (Expt 3) six mice were each fed an 800 mg/kg diet of CDDO Methyl Amide (TP-224) for two days.

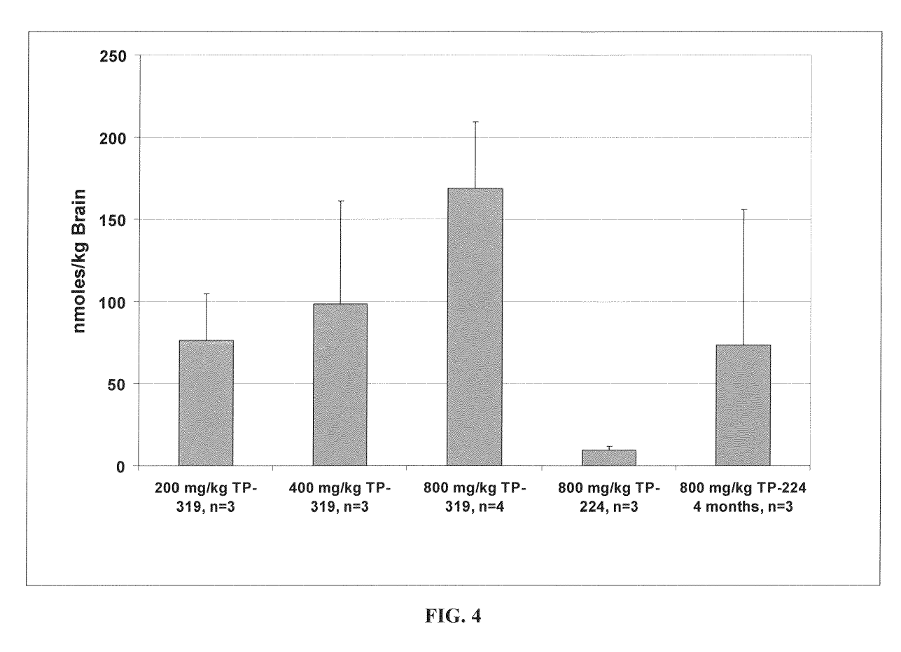

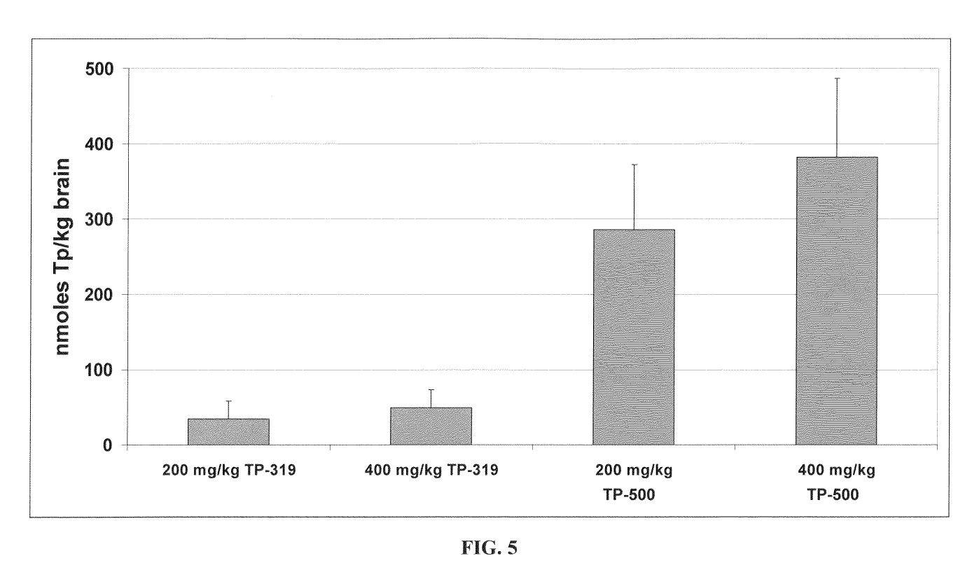

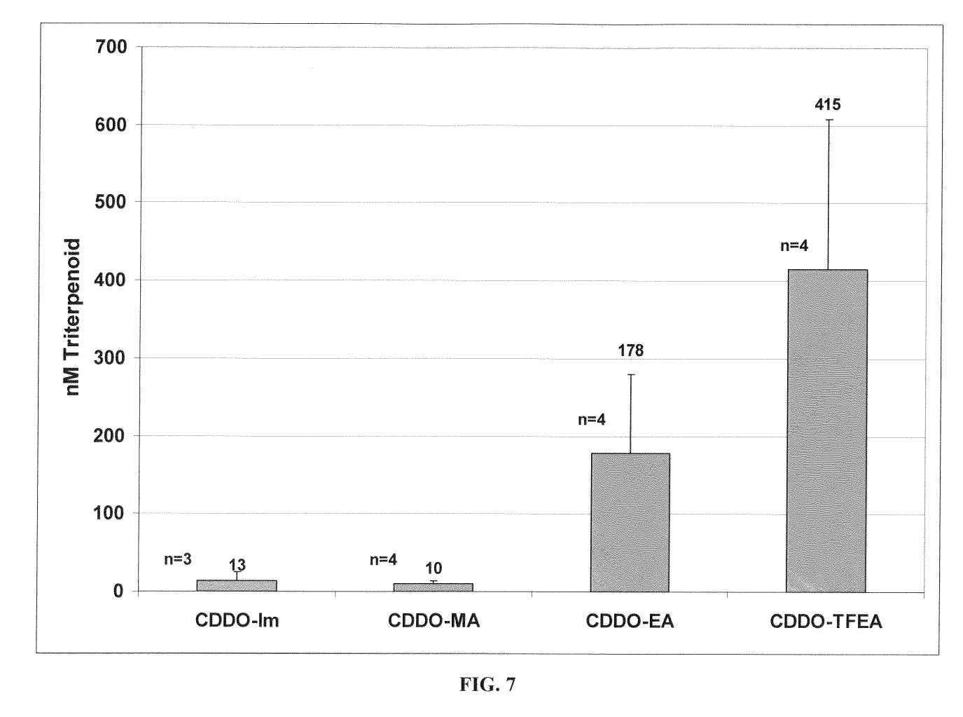

As shown in FIG. 3, feeding CDDO-EA (TP-319) for two days results in higher brain levels than when the mice are fed CDDO-MA (TP-224). FIG. 5 shows that CDDO-TFEA (TP-500) is detected at higher levels in mouse brain than is CDDO-EA (TP-319). The effects are dose responsive. FIG. 4 shows that the brain levels of CDDO-EA (TP-319) are dose responsive and higher than for CDDO-MA (TP-224). The brain levels of triterpenoids detected in gavaged CD-1 mice also varied with the structure of the triterpenoid (FIG. 7).

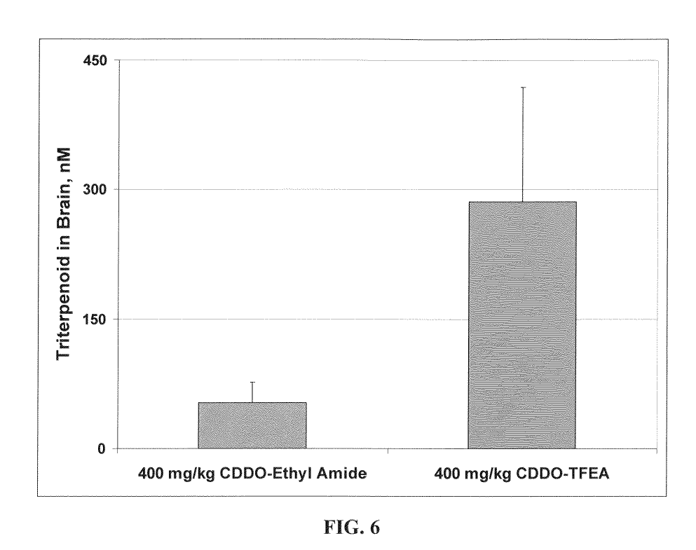

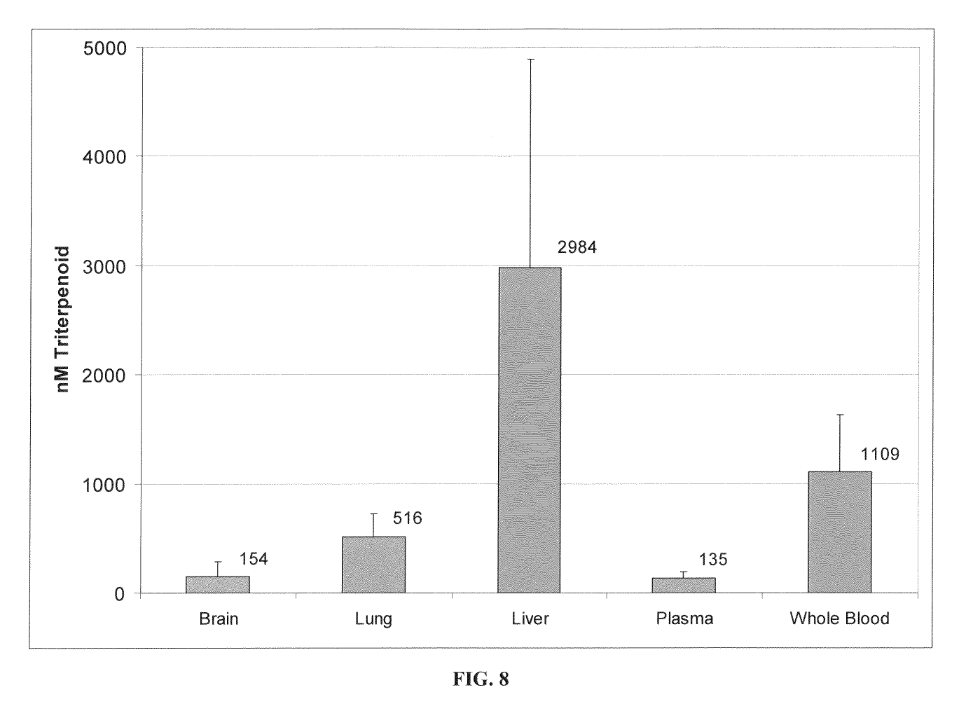

The ability of synthetic triterpenoids to remain in the brain also varies according to their structure. As shown in FIG. 6, the brain levels of CDDO-TFEA (TP-500) remain significantly higher than CDDO-EA (TP-319). Furthermore, as shown in FIGS. 8 and 9, the relative concentration in the brain of gavaged mice was higher for CDDO-TFEA than for CDDO-EA. FIGS. 8 and 9 also show the distribution of CDDO-EA (TP-319) and CDDO-TFEA (TP-500), respectively, in the following CD-1 mouse tissues: brain, lung, liver, plasma, and whole blood.

Experiments using CDDO-Me (RTA-402) were also conducted on 1 male and 1 female cynomolgus monkey (origin: Vietnam) between the ages of 2 and 3 years and weighing approximately 1.7 kg. Each received the test article (CDDO-Me in sesame oil) at 75 mg/kg/day via oral gavage administered at a volume of 5 mL/kg on Days 1, 2, and 3. Individual doses were based on the most recently obtained body weights. Blood samples were collected from the femoral artery/vein for determination of the plasma concentrations of the test article at 0.5, 1.5, 3, and 12 hours after dosing on Days 1 and 2 and at 0.5, 1.5, and 3 hours after dosing (±0.5 hour) on Day 3.

At the termination of the study (approximately three hours after dosing on Day 3), all animals were euthanized and tissues collected. Samples (approximately 1 g or greater) of the adipose tissue, brain, colon, cheek pouch (buccal mucosa), heart, ileum, kidney, liver, lung, mammary glands, ovaries, pancreas, prostate, and bone marrow from the femur (as much as possible) were collected and frozen at approximately −20° C. for analysis for the presence of the test article. All other tissues and organs were discarded.

Table 1 shows the average distribution of CDDO-Me (RTA-402) in tissues of cynomolgus monkeys after 3 days of oral dosing at 1800 mg/m² (vehicle is sesame seed oil).

TABLE 1
Average Distribution of CDDO-Me in Monkey Tissue

| Organ | CDDO-Me (nM) |
|---|---|
| Breast | 4,389 |
| Lung | 4,992 |
| Colon | 4,467 |
| Prostate | 4,446 |
| Ovary | 2,115 |
| Kidney | 1,603 |
| Liver | 926 |
| Brain | 215-447 |

In Table 1 nM is ng/mL×1000/505.8, where 505.8 is the molecular weight of RTA-402 using the approximation that the density of the tissue is that of water. In the case of brain tissue, Table 1 shows the range of results obtained.

### Example 3

### In Vivo Results from EAE Studies

The mice used for these studies were female and either wild type or heterozygotes for the Tgf-b1 gene. The latter have a more accelerated course of disease (yet are equally protected by triterpenoid treatment). The mouse strain used for these studies includes either a mixed SvEV 129×C56BL/6 or a pure SvEV129 strain.

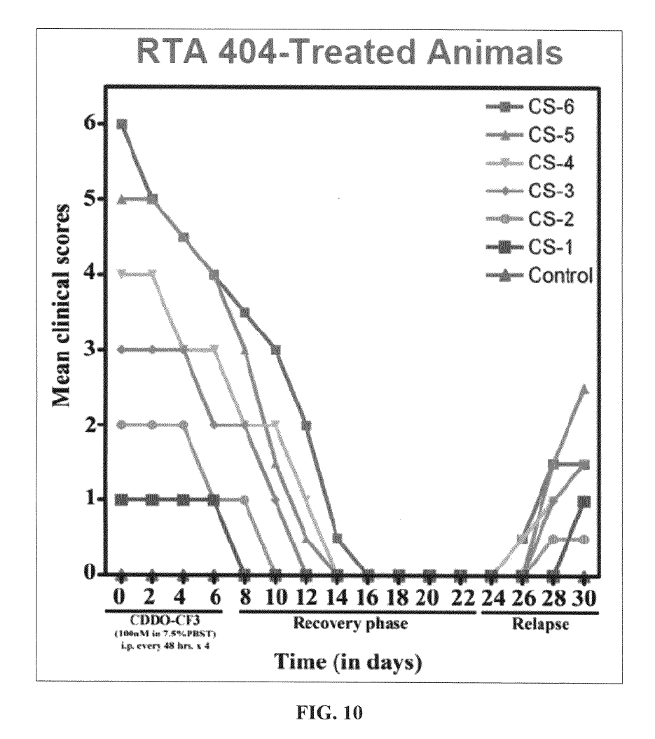

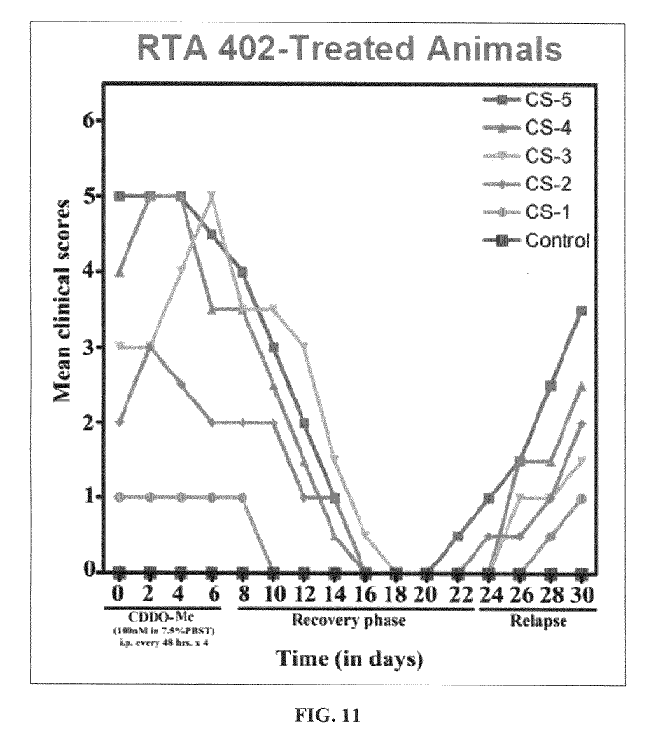

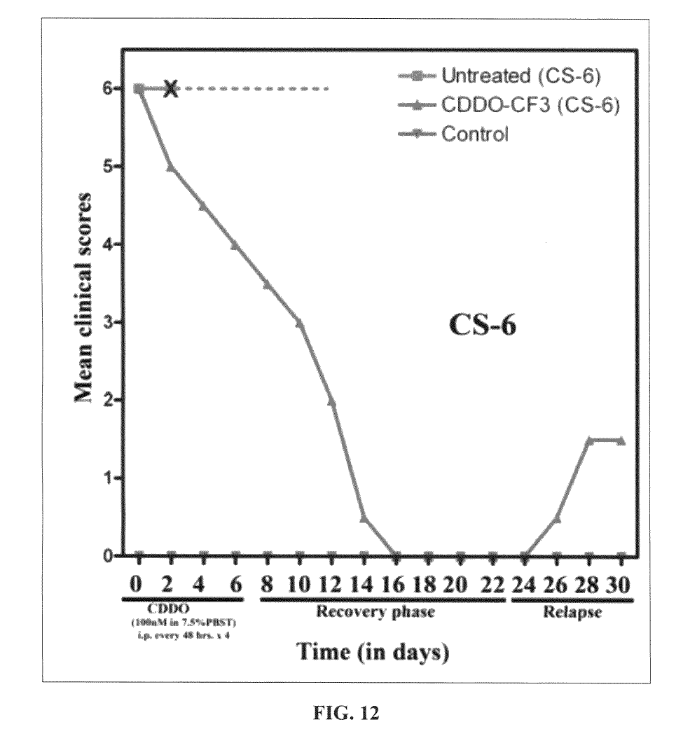

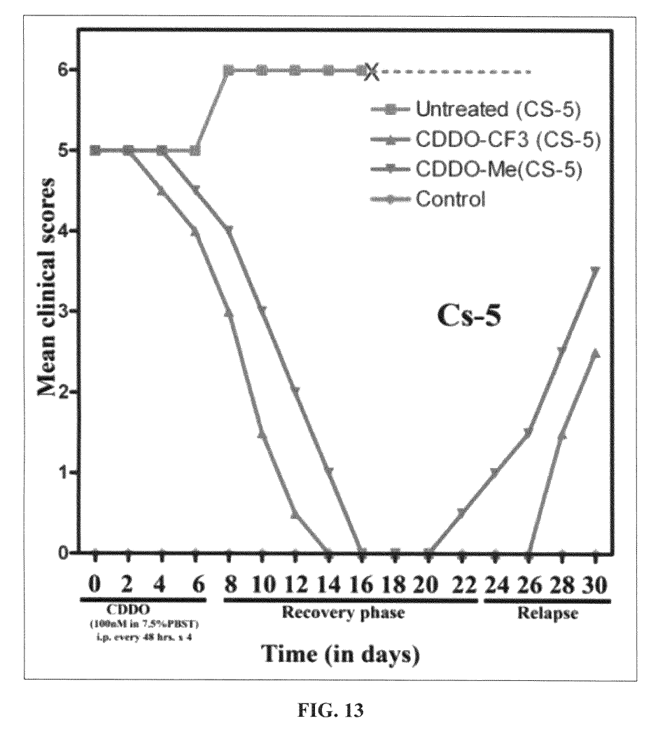

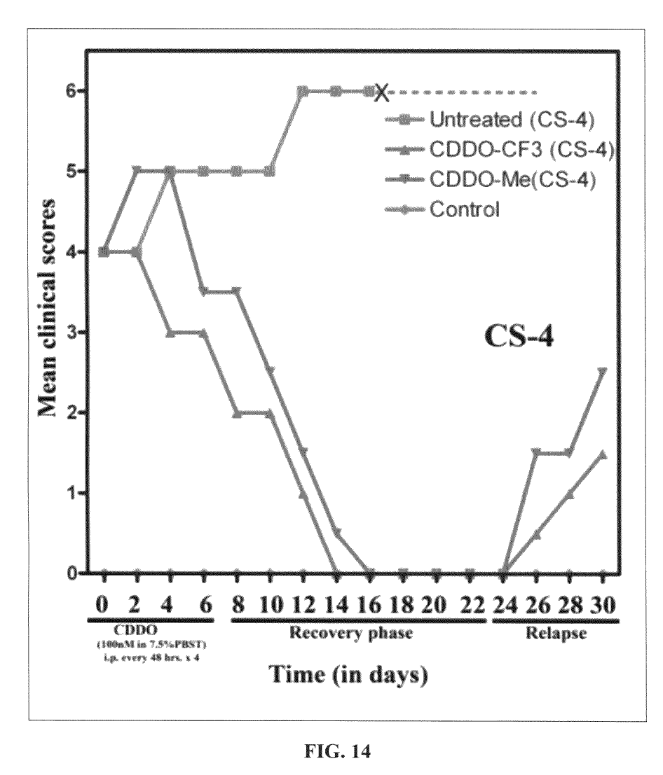

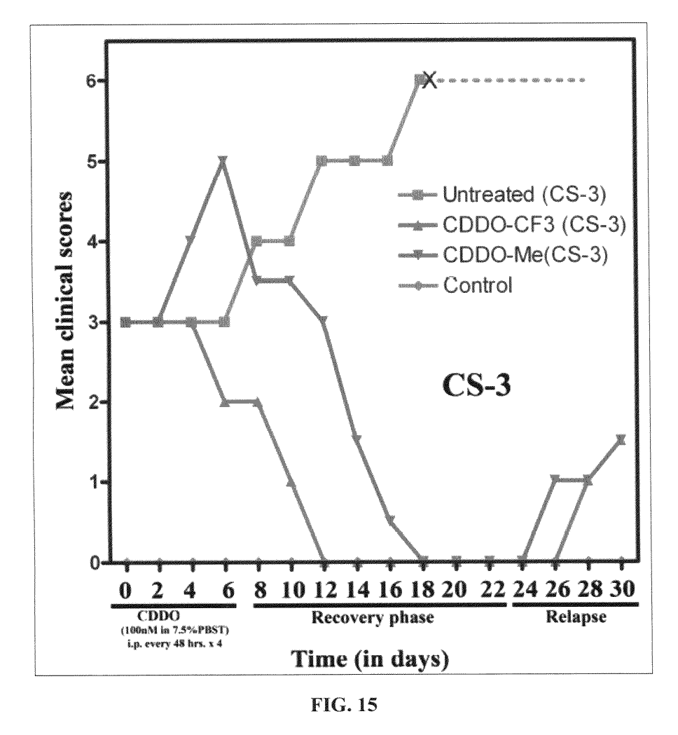

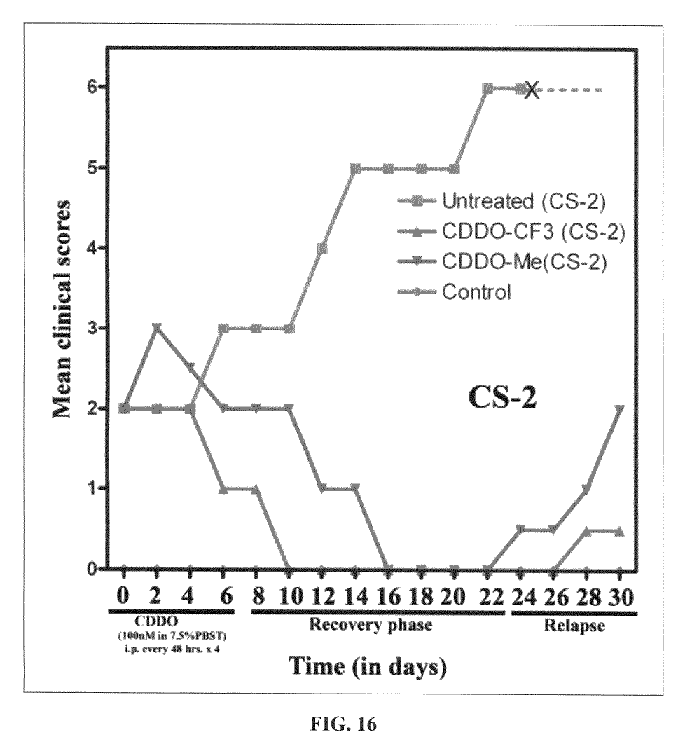

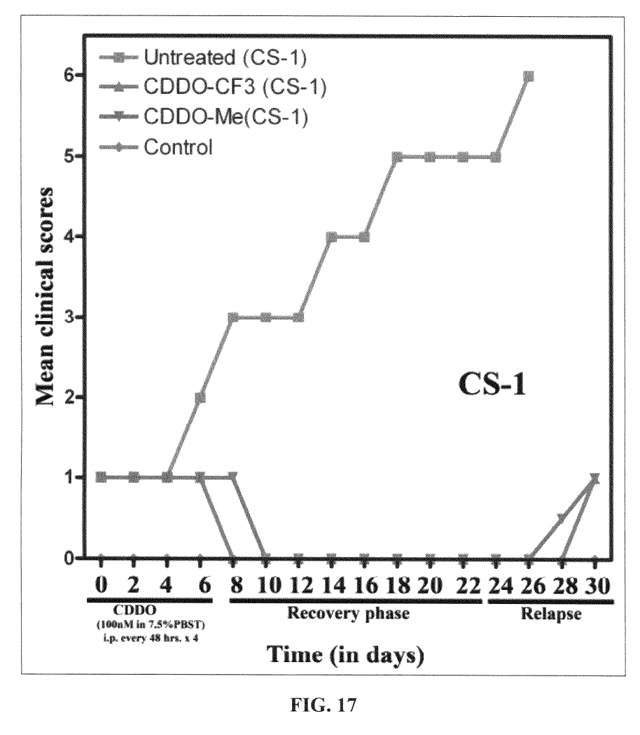

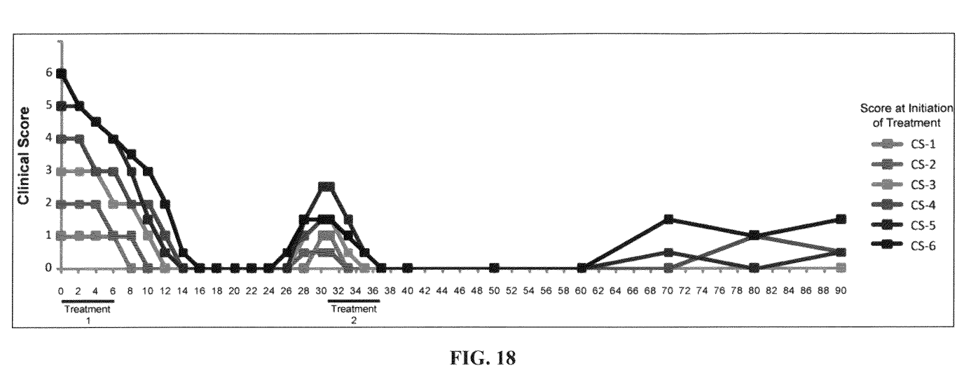

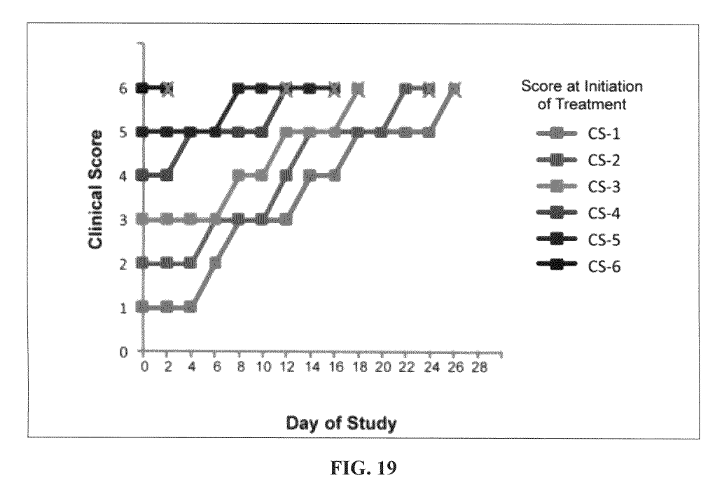

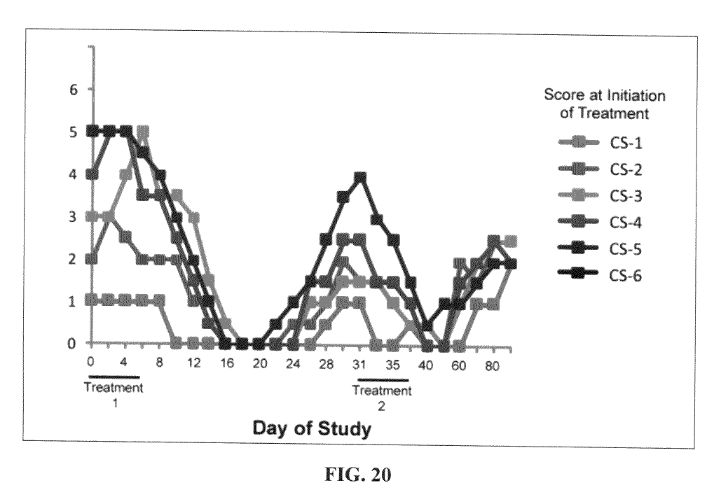

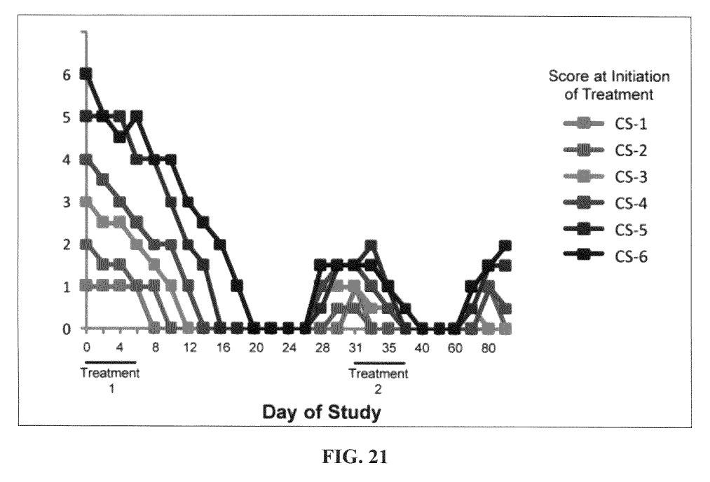

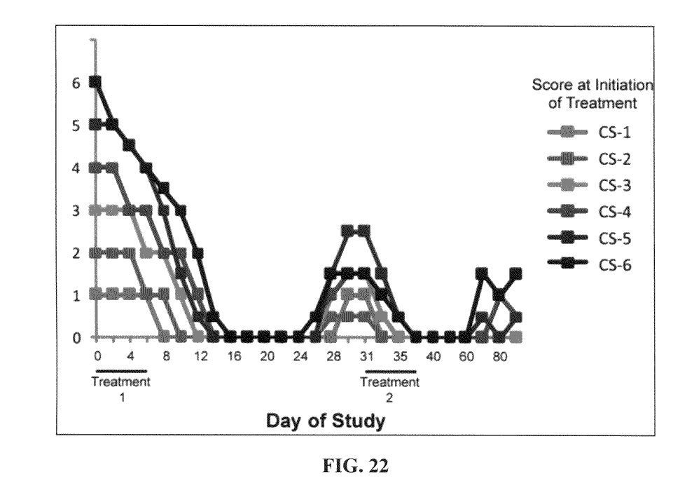

Slight variations in protocol were used across all studies to evaluate and optimize the activity. For the studies correlating with FIGS. 10-22, animals were injected with the following:

CFA: 100 microliter incomplete Freund's Adjuvant+8 mg/ml mycobacterium Tuberculosis+100 microliter PBS

PTX: Pertussis Toxin 200 ng in 100 microliter PBS once at the time of immunization and once after 48 hrs

In EAE-induced animals, MOG was administered, and approximately 18-21 days later, scores of 5 to 6 were attained (complete hind limb paralysis to complete paralysis)

In treatment studies, including histology, cytokine, and molecular studies, animals treated with all agents were given 10 nanomoles (RTA 404-0.29 mg/kg, RTA 402-0.25 mg/kg, RTA 405-0.26 mg/kg) IP on a QOD×4 or 5 schedule (human equivalent dose ˜1.5 mg)

Clinical assessment studies: Treated animals received 4 injections that began once animals achieved various scores

Histology and molecular studies: RTA 404-treated animals received 4 injections that began once animals achieved scores of 5. Once scores returned to 0, animals were sacrificed. Controls were sacrificed at scores of 5.

Cytokine studies: RTA 404-treated animals received 5 injections that began once animals achieved scores of 5, and once scores returned to 0, animals were sacrificed. Controls were sacrificed at scores of 6.

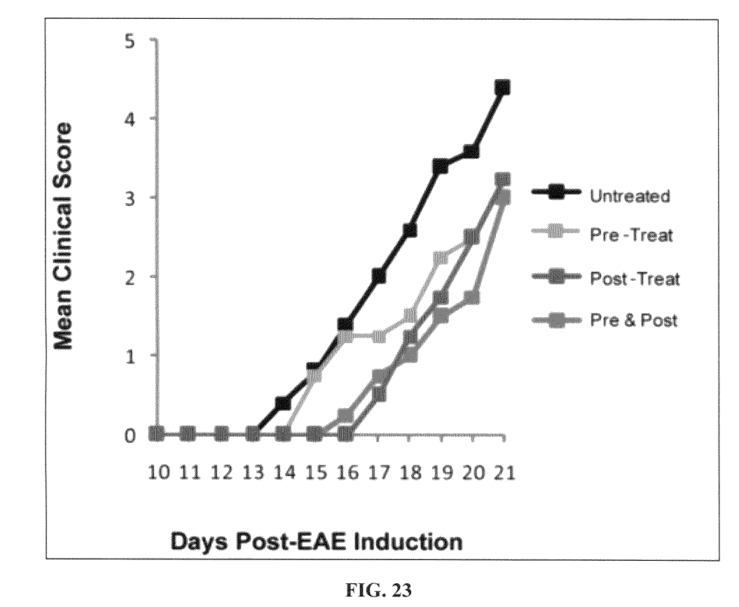

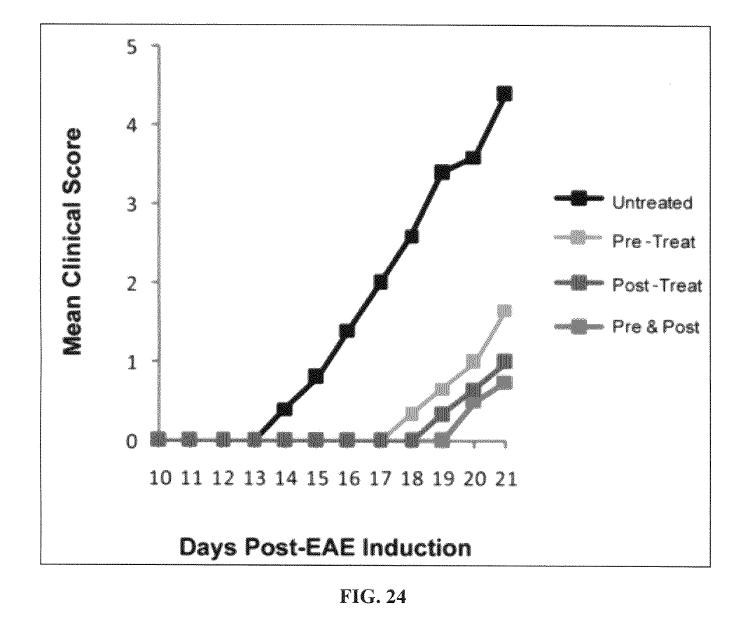

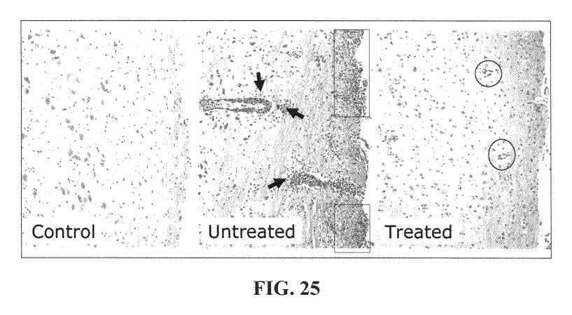

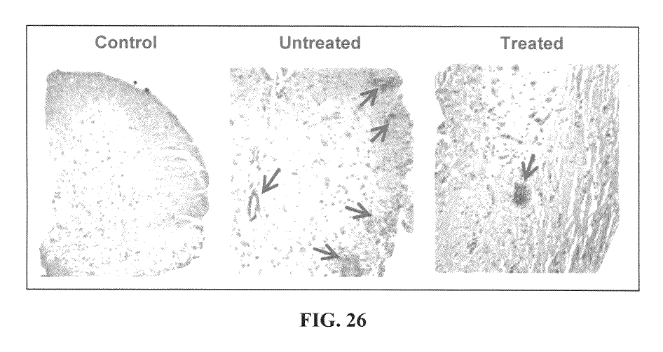

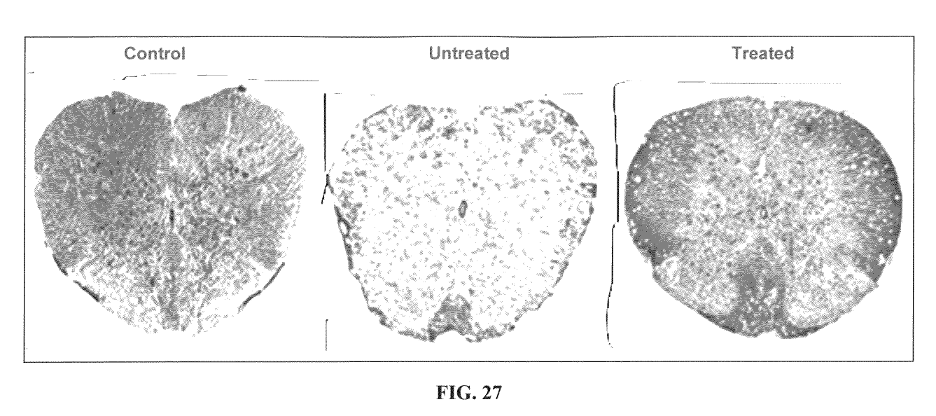

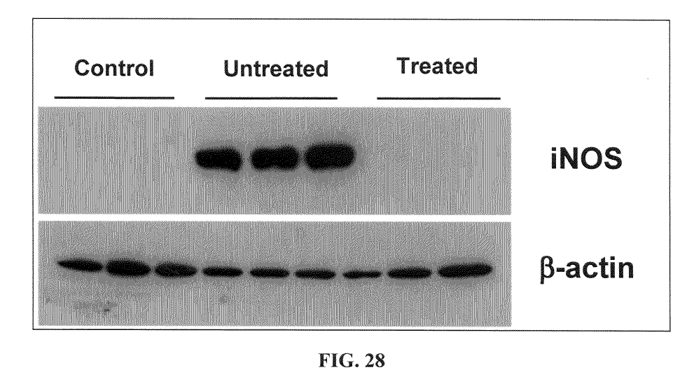

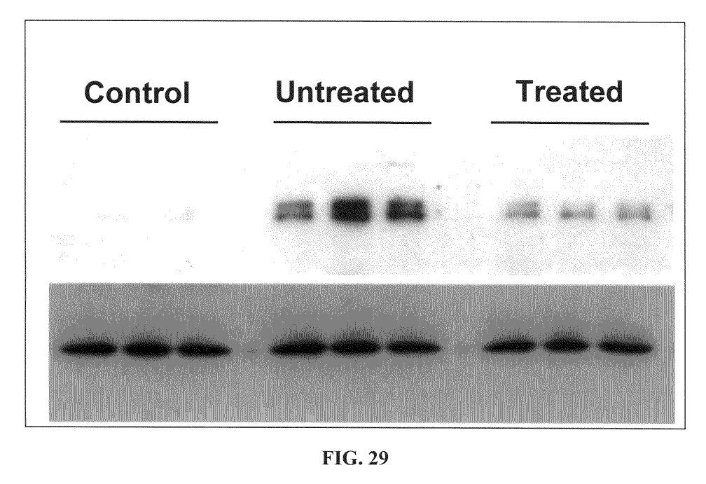

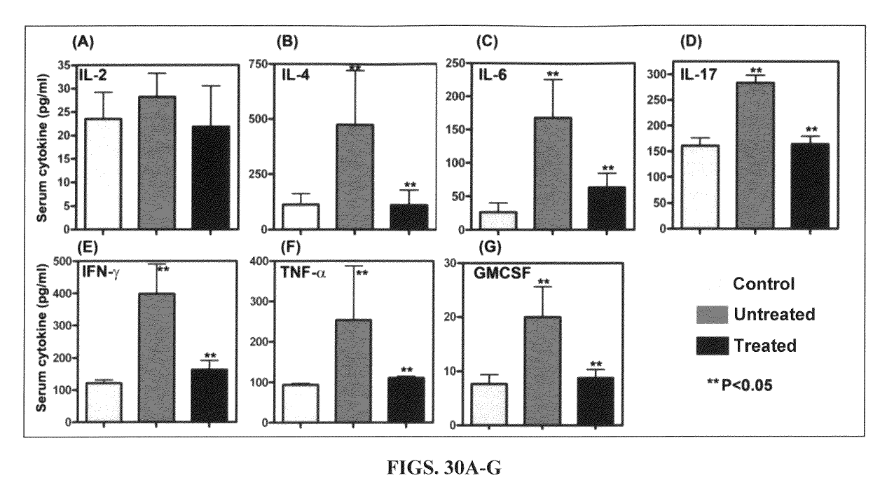

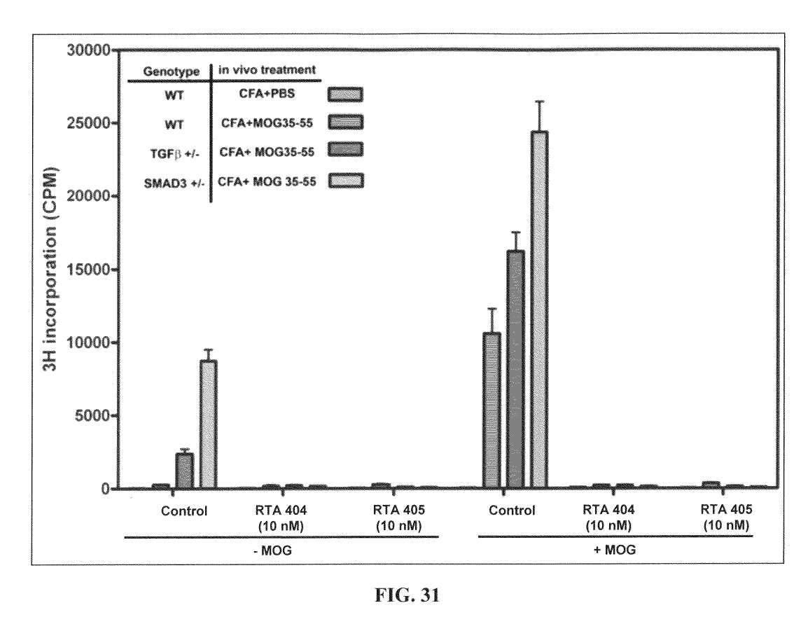

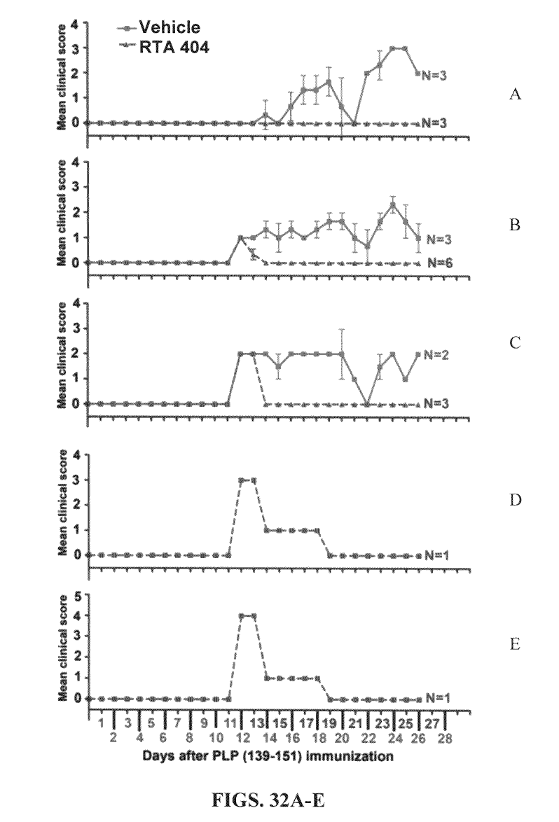

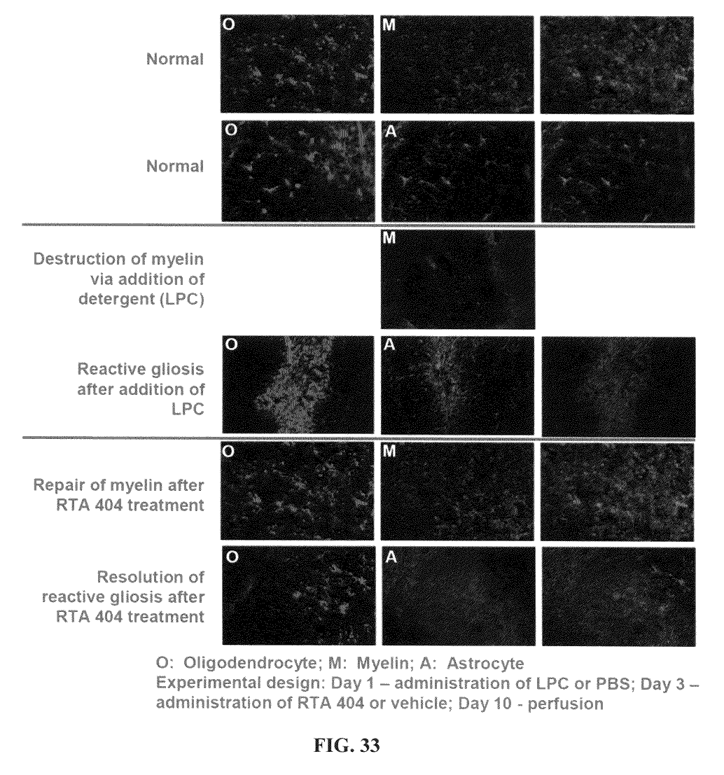

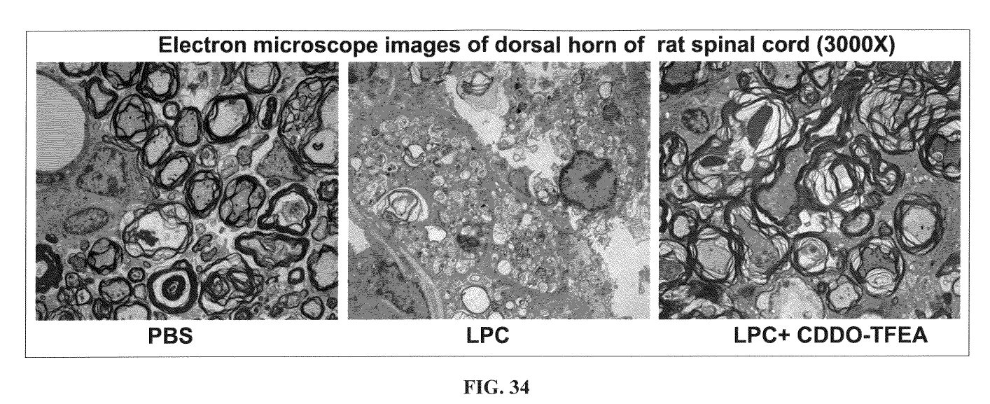

In prophylactic studies (FIGS. 23-24) the following design was used:

Day-1: 100 nanomoles IP RTA 404 (2.9 mg/kg) or RTA 402 (2.5 mg/kg) in pre and pre/post groups

Day 0: MOG

Day 1: 100 nanomoles IP RTA 404 (2.9 mg/kg) or RTA 402 (2.5 mg/kg) in post and pre/post groups

Day 3: 100 nanomoles IP RTA 404 (2.9 mg/kg) or RTA 402 (2.5 mg/kg) in post and pre/post groups

Day 5: 100 nanomoles IP RTA 404 (2.9 mg/kg) or RTA 402 (2.5 mg/kg) in post and pre/post groups

As shown in FIGS. 10-17, the various synthetic triterpenoids, e.g., CDDO-TFEA (RTA 404), CDDO-Me (RTA-402) and CDDO-EA (RTA-405), induce full recovery of mice in a rapidly progressive experimental autoimmune encephalomyelitis (EAE) model. Animals (n=2/group) of varying clinical score (CS) were immunized with myelin oligodendrocyte glycoprotein (MOG) and treated intraperitoneally (IP) with 100 nmol in a volume of 50-100 μL (˜2.8 mg/kg) of synthetic triterpenoid every other day for a total of four times (Q2D×4 schedule). Further experiments showed that lower doses (10 nmol) of these TPs were also effective. A CS of 0 indicates no symptoms, and score of 6 indicates quadriplegia. The drugs may not be killing immune effector cells, which may explain the relapse. Relapsed animals do respond to additional treatment (data not shown). It was shows that untreated animals develop severe paralysis and die within days of developing quadriplegia. Treated animals respond within a few days and fully recover to absence of any paralysis.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 6,025,395
U.S. Pat. No. 6,326,507
U.S. Pat. No. 6,552,075
U.S. Pat. No. 6,974,801
Abraham and Kappas, Free Radic. Biol. Med., 39(1):1-25, 2005.
Ahmad et al., J. Biol. Chem., 281:3576-3579, 2006.
Akiyama et al., Alzheimer Dis. Assoc. Disord., 14(1):S47-53, 2000.
Angulo et al., Eur. J. Immunol., 30:1263-1271, 2000.
Araujo et al., J. Immunol., 171(3):1572-1580, 2003.
Arend and Dayer, Arthritis Rheum., 38:151-160, 1995.
Arend et al., Annu. Rev. Immunol., 16:27-55, 1998.
Autenrieth et al., Infect. Immun., 62:2590-2599, 1994.
Bach, Hum. Immunol., 67(6):430-432, 2006
Bagasra et al., Proc. Natl. Acad. Sci. USA, 92:12041-12045, 1995.
Balkwill et al., Cancer Cell, 7:211-217, 2005.
Ball, Ann. Rheum. Dis., 30:213-223, 1971.
Beal, Curr. Opin. Neurobiol., 6:661-666, 1996.
Bendzen et al., Scand. J. Rheumatol., 28:599-606, 1988.
Blumberg et al., Arthritis Rheum., 7:93-97, 1964.
Bore et al., Acta Cryst., C58:199-200, 2002.
Botoman et al., Am. Fam. Physician, 57(1):57-68, 1998.
Brandt et al., Arthritis Rheum., 43:1346-1352, 2000.
Braun et al., Arthritis Rheum., 42:2039-2044, 1999.
Brewerton et al., Lancet., 1:904-907, 1973a.
Brewerton et al., Lancet., 1:956-957, 1973b.
Bronte et al., Trends Immunol., 24:302-306, 2003.
Brown and DuBois, J. Clin. Oncol., 23:2840-2855, 2005.
Brynskov et al., N. Engl. J. Med., 321(13):845-850, 1989.
Burger and Dayer, Neurology, 45 (6S-6):S39-43, 1995.
Cai et al., Nat. Med., 11(2):183-190, 2005.
Calin and Taurog, In: The Spondylarthritides, Calin et al. (Eds.), Oxford, UK. Oxford University Press, 179, 1998.
Cann et al., Gut., 24(12):1135-1140, 1983.
Chauhan and Chauhan, Pathophysiology, 13(3):171-181, 2006.
Chomarat et al., Arthritis Rheum., 38:1046-1054, 1995.
Coyle and Puttfarcken, Science, 262:689-695, 1993.
Crowell et al., Mol. Cancer Ther., 2:815-823, 2003.
Culver et al., Science, 256:1550-1552, 1992.
de Waal et al., J. Exp. Med., 174:1209-1220, 1991.
Dickerson et al., Prog Neuropsychopharmacol Biol. Psychiatry. Mar. 6, 2007.
Dinarello, Int. Rev. Immunol., 16:457-499, 1998.
Dinkova-Kostova et al., Proc. Natl. Acad. Sci. USA, 102(12):4584-4589, 2005.
Dionne et al., Clin. Exp. Immunol., 112(3):435-442, 1998.
Doran et al., J. Rheumatol., 30(2):316-320, 2003.
Drossman et al., Dig. Dis. Sci., 38(9):1569-1580, 1993.
Drossman et al., Gastroenterol., 112(6):2120-2137, 1997.
Dudhgaonkar et al., Eur. J. Pain, 10(7):573-9, 2006.
Eikelenboom et al., Glia, 40(2):232-239, 2002.
Ettehadi et al., Clin. Exp. Immunol., 96(1):146-151, 1994.
Everhart et al., Gastroenterol., 100(4):998-1005, 1991.
Fearon and Locksley, Science, 272(5258):50-53, 1996.
Feldtkeller et al., Rheumatol. Int., 23(2):61-66, 2003.
Firestein et al., Arthritis Rheum., 37:644-652, 1994.
Forstermann, Biol. Chem., 387:1521, 2006.
Fujikawa et al., Ann. Rheum. Dis., 54:318-320, 1995.
Funakoshi et al., Digestion, 59(1):73-78, 1998.
Galley and Webster, Br. J. Anaesth., 77:11-16, 1996.
Gehrmann et al., Glia, 15(2):141-151, 1995.
Genain and Nauser, J. Mol. Med., 75:187-197, 1997.
Gladman et al., Br. J. Rheumatol., 22:675-679, 1995.
Gladman et al., J. Med., 62:127-141, 1987.
Gladman, Rheum. Dis. Clin. North Am., 18:247-256, 1992.
Gold et al., Brain, 129(8):1953-1971, 2006.
Goodman et al., Kidney Int., 72(8):945-953, 2007.
Graeber et al., Glia, 40(2):252-259, 2002.
Greten et al., Cell, 118:285-296, 2004.
Griffin et al., Proc. Natl. Acad. Sci. USA, 86(19):7611-7615, 1989.
Guttridge et al., Mol. Cell. Biol., 19:5785-5799, 1999.
Gwee et al., Gut., 44(3):400-406., 1999.
Hahn and Tsao, In: Dubois' Lupus Erythematosus, 4^{th} Ed, Wallace and Hahn (Eds.), Lea and Febiger, Philadelphia, 195-201, 1993.
Handbook of Pharmaceutical Salts: Properties, Selection and Use (Stahl & Wermuth, Eds.), Verlag Helvetica Chimica Acta, 2002.
Hannum et al., Nature, 343:336-340, 1990.
Hanson et al., BMC Medical Genetics, 6(7), 2005.
Harrison and Symmons et al., Ann. Rheum. Dis., 57(6):375-377, 1998.
Harrison et al., J. Rheumatol., 25(12):2324-2330, 1998.
Hart et al., Immunology, 84:536-542, 1995.
Hinz et al., Mol. Cell Biol., 19:2690-2698, 1999.
Hohler et al., Arthritis Rheum., 41:1489-1492, 1998.
Hohler et al., J. Invest. Dermatol., 109:562-565, 1997.
Honda et al., Bioorg. Med. Chem. Lett., 12:1027-1030, 2002.
Honda et al., Bioorg. Med. Chem. Lett., 19:2711-2714, 1998.
Honda et al., Bioorg. Med. Chem. Lett., 9:3429-3434, 1999.
Honda et al., J. Med. Chem., 43:1866-1877, 2000a.
Honda et al., J. Med. Chem., 43:4233-4246, 2000b.
Honda et al., Med. Chem. Lett., 7:1623-1628, 1997.
Honda et al., Org. Biomol. Chem., 1:4384-4391, 2003.
Horwitz and Fisher, N. Engl. J. Med., 344(24):1846-1850, 2001.
Huang et al., Cancer Res., 54:701-708, 1994.
Ishikawa et al., Circulation, 104(15): 1831-1836, 2001.
Ishizawa and Dickson, J. Neuropathol. Exp. Neurol., 60(6):647-657, 2001.
Ito et al., Cell Growth Differ., 11:261-267, 2000.
Jacob et al., Proc. Natl. Acad. Sci. USA, 87:1233-1237, 1990.
Jailwala et al., Ann. Intern. Med., 133(2):136-147, 2000.
Jarvis, Curr. Opin. Rheumatol., 10(5):459-467, 1998.
Jarvis, Pediatr. Ann., 31(7):437-446, 2002.
Jones et al., Br. J. Rheumatol., 33(9):834-839, 1994.
Jonsson et al., Br. J. Rheumatol., 32(7):578-581 1993.
Jonsson et al., Oral Dis., 8(3):130-140, 2002.
Jonsson et al., Trends Immunol., 22(12):653-654, 2001.
Joyce et al., J. Biol. Chem., 274:25245-25249, 1999.
Juedes et al., J. Immunol., 164(1):419-426, 2000.
Kahle et al., Ann. Rheum. Dis., 51:731-734, 1992.
Kaltschmidt et al., Proc. Natl. Acad. Sci. USA, 94:2642-2647, 1997.
Kawakami et al., Brain Dev., 28(4):243-246, 2006.
Kellow and Phillips, Gastroenterol., 92(6):1885-1893, 1987.
Kendall-Tackett, International Breastfeeding J., 2(6), 2007.
Kendall-Tackett, Trauma Violence Abuse, 8(2):117-126, 2007.
Khan et al., J. Neurochem., 71:78-87, 1998.
Khan et al., Toxicol. Applied Pharmacol., 103:482-490, 1990.
Kim et al., Mol. Cancer. Ther., 1: 177-184, 2002.
Konopleva et al., Blood, 99:326-335, 2002.
Kortylewski et al., Nat. Med., 11: 1314-1321, 2005.
Kotake et al., Infect. Immun., 67:2682-2686, 1999.
Kotzin and O'Dell, In: Samler's Immunologic Diseases, 5^{th} Ed., Frank et al. (Eds.), Little Brown & Co., Boston, 667-697, 1995.
Kotzin, Cell, 85:303-306, 1996.
Kruger et al., J Pharmacol Exp Ther., Sep. 7, 2006.
Kruger et al., J. Pharmacol. Exp. Ther., 319(3):1144-1152, 2006.
Kuboyama, Kurume Med. J., 45(1):33-37, 1998.
Lahesmaa et al., J. Immunol., 148:3079-3085, 1992.
Lee et al., Glia., 55(7):712-22, 2007.
Lencz et al., Mol. Psychiatry, 12(6):572-80, 2007.
Liby et al., Cancer Res., 65:4789-4798, 2005.
Lipsky, In: Harrison's principles of internal medicine, Fauci et al. (Eds.), 14^{th} Ed., NY, McGraw-Hill, 1880-1888, 1998.
Liu et al., FASEB J., 20(2):207-216, 2006.
Lo et al., Curr. Dir. Autoimmun., 1:226-246, 1999.
Lugering et al., Ital. J. Gastroenterol. Hepatol., 30(3):338-344, 1998.
Luo et al., J. Clin. Invest., 115(10):2625-2631, 2005.
Lynn and Friedman, N. Engl. J. Med., 329(26):1940-1945, 1993.
Macatonia et al., J. Immunol., 150:3755-3765, 1993.
March's Advanced Organic Chemistry: Reactions, Mechanisms, and Structure (March's Advanced Organic Chemistry), Smith and March (Eds.), 2007.
Marsal et al., Rheumatology, 38:332-337, 1999.
Mazzoni et al., J. Immunol., 168:689-695, 2002.
McAlindon et al., Gut, 42(2):214-219, 1998.
McGeer and McGeer, Brain Res. Brain Res. Rev., 21:195-218, 1995.
McGeer et al., Neurology, 19:331-338, 1996.
McGonagle et al., Arthritis Rheum., 41:694-700, 1998.
McGonagle et al., Curr. Opin. Rheumatol., 11:244-250, 1999.
McIver et al., Pain., 120 (1-2):161-9, 2005.
Mease et al., Lancet, 356:385-390, 2000.
Merrill and Benvenist, Trends Neurosci., 19:331-338, 1996.
Mertz et al., Gastroenterol., 118(5):842-848, 2000.
Minns et al., Gastroenterology 127:119-26, 2004.
Mix et al., Mol Pharmacol 65:309-318, 2004.
Moll and Wright, Ann. Rheum. Dis., 32:181-201, 1973.
Moll and Wright, Semin. Arthritis Rheum., 3:55-78, 1973.
Morris et al., J. Mol. Med., 80(2):96-104, 2002.
Morse and Choi, Am. J. Respir. Crit. Care Med., 172(6):660-670, 2005.
Morse and Choi, Am. J. Respir. Crit. Care Med., 27(1):8-16, 2002.
Na and Surh, Mol. Carcinog., 45:368-380, 2006.
Nath et al., Neurology, 66(1):149-150, 2006.
Neal et al., BMJ., 314(7083):779-782, 1997.
Nichols, Drug News Perspect., 17(2):99-104, 2004.
Nielen et al., Arthritis Rheum., 50(2):380-386, 2004.
Nishino et al., Cancer Res., 48:5210-5215, 1988.
Ohnishi et al., Int. Immunol., 6:817-830, 1994.
Owens, Adv. Neurol., 98:77-89, 2006.
Pall, Med. Hypoth., 69:821-825, 2007.
Partsch et al., Br. J. Rheumatol., 24:518-523, 1997.
Pimentel et al., Am. J. Gastroenterol., 95(12):3503-3506, 2000.
Place et al., Clin. Cancer Res., 9:2798-2806, 2003.
Pociot et al., Scand. J. Immunol., 42(4):501-504, 1995.
Prieur et al., Lancet., 2:1240-1242, 1987.
Rantapaa-Dahlqvist et al., Arthritis Rheum., 48(10):2741-2749, 2003.
Reimund et al., Eur. J. Clin. Invest., 28(2):145-150, 1998.
Ribbens et al., Eur. Cytokine Netw., 11:669-676, 2000.
Rogers et al., Neurobiol Aging, 9(4):339-349, 1988.
Rogler and Andus, World J. Surg., 22(4):382-389, 1998.
Rooney et al., Rheumatol. Int., 10:217-219, 1990.
Ross et al., Nutr. Neurosci., 6(5):277-81, 2003.
Rostom et al., Ann. Intern. Med., 146, 376-389, 2007.
Rothstein, Med. Clin. North Am., 84(5):1247-1257, 2000.
Ruster et al., Scand. J. Rheumatol., 34(6):460-3, 2005.
Sacerdoti et al., Curr Neurovasc Res. 2(2):103-111, 2005.
Saiki et al., Scand. J. Gastroenterol., 33(6):616-622, 1998.
Salomonsson and Jonsson, Arthritis Rheum., 48(11):3187-3201, 2003.
Salomonsson et al., Scand. J. Immunol., 55(4):336-342, 2002.
Salvarani et al., Curr. Opin. Rheumatol. 1998; 10:299-305, 1998.
Salvemini et al., J. Clin. Invest., 93:1940-1947, 1994.
Sandler, Gastroenterol., 99(2):409-415, 1990.
Sarchielli et al., Cephalalgia, 26(9):1071-1079, 2006.
Satoh et al., Proc. Natl. Acad. Sci. USA, 103(3):768-773, 2006.
Schellekens et al., Arthritis Rheum., 43(1):155-163, 2000.
Schlaak et al., Clin. Exp. Rheumatol., 14:155-162, 1996.
Schlaak et al., Eur. J. Immunol., 22:2771-2776, 1992.
Schlosstein et al., NE J. Medicine, 288:704-706, 1973.
Schreiber, Neth. J. Med., 53 (6):S24-31, 1998.
Schulz et al., Antioxid. Redox. Sig., 10:115, 2008.
Shishodia et al., Clin. Cancer Res., 12(6):1828-1838, 2006.
Sieper and Braun, Arthritis Rheum., 38:1547-1554, 1995.
Simon et al., Clin. Exp. Immunol., 94:122-126, 1993.
Simon et al., Proc. Natl. Acad. Sci. USA, 91:8562-85666, 1994.
Simonian and Coyle, Annu. Rev. Pharmacol. Toxicol., 36:83-106, 1996.
Sinha et al., Cancer Res., 67:4507-4513, 2007.
Stack et al., Lancet, 349(9051):521-524, 1997.
Stewart et al., Neurology, 48:626-632, 1997.
Strejan et al., J. Neuroimmunol., 7:27, 1984.
Suh et al., Cancer Res 63:1371-1376, 2003.
Suh et al., Cancer Res., 58:717-723, 1998.
Suh et al., Cancer Res., 59(2):336-341, 1999.
Szabo et al., Nature Rev. Drug Disc., 6:662-680, 2007.
Takahashi et al., Cancer Res., 57:1233-1237, 1997.
Talley et al., Gastroenterol., 109(6):1736-1741, 1995.
Tamir and Tannenbaum, Biochim. Biophys. Acta., 1288:F31-F36, 1996.
Targan et al., N. Engl. J. Med., 337(15):1029-1035, 1997.
Thimmulappa et al., Biochem. Biophys. Res. Commun., 351:883-999, 2006.
Third Report of the National Cholesterol Education Program Expert Panel on Detection, Evaluation and Treatment of High Blood Cholesterol in Adults (Adult Treatment Panel III, or ATP III), National Institutes of Health, 2001, NIH Publication No. 01-3670.
Touzani et al., J. Neuroimmunol., 100 (1-2):203-215, 1999.
Tumlin et al., Am. J. Cardiol., 98 (6A):14K-20K, 2006.
van den Berg, Semin. Arthritis Rheum., 30 (5S-2):7-16, 2001.
van Dullemen et al., Gastroenterol., 109(1):129-135, 1995.
van Hogezand and Verspaget, Drugs, 56(3):299-305, 1998.
Virley, J. Amer. Soc. Exper. NeuroTherap., 2:638-649, 2005.
Vodovotz et al., In; Handbook of Experimental Immunology, Volumes I-IV, 1996.
Wardle, Nephrol. Dial. Transplant., 16(9):1764-8, 2001.
Warrington et al., Arthritis and Rheumatism, 44:13-20, 2001.
Wermuth and Stahl, In: Pharmaceutical Salts: Properties, Selection and Use—A Handbook, Verlag Helvetica Chimica Acta, 2002.
Weyand and Goronzy, Ann. NY Acad. Sci., 987:140-149, 2003.
Whitehead et al., Gastroenterol., 98 (5 Pt 1):1187-1192, 1990.
Williams et al., Clin. Neurosci., 2 (3-4):229-245, 1994.
Wolinsky et al., Ann. Neurol. 61(1):14-24, 2007.
Wordsworth, In: Genes and Arthritis, Brit. Medical Bulletin, 51:249-266, 1995.
Wright, Ann. Rheum. Dis., 15:348-356, 1956.
Wright, Clin. Orthop. Related Res., 143:8-14, 1979.
Xanthou et al., Arthritis Rheum., 44(2):408-418, 2001.
Yates et al., Cancer Res., 66(4): 2488-2494, 2006.
Yates et al., Mol. Cancer Ther., 6(1):154-162, 2007.
Yin et al., Arthritis Rheum., 40:1788-1797, 1997.
Yin et al., Rheumatology, 38:1058-1067, 1999.
Yoh et al., Kidney Int., 60(4):1343-1353, 2001.
Yore et al., Mol. Cancer Ther., 5:3232-3239, 2006.
Yu and Kensler, Mutat. Res., 591:93-102, 2005.
Yu et al., Nat. Rev. Immunol., 7:41-51, 2007.
Zhou et al., Am. J. Pathol., 166(1):27-37, 2005.
Zhou et al., Cancer Sci., 98:882-889, 2007.
Zingarelli et al., J. Immunol., 171(12):6827-6837, 2003.

1-25. (canceled)

26. A compound having the structure: 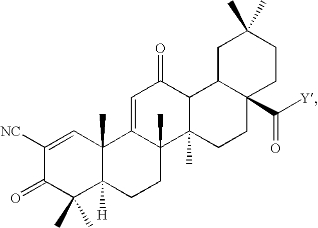
wherein Y′ is ethylamino or a heteroatom-substituted C₁-C₅-alkylamino having at least one fluorine atom; or
pharmaceutically acceptable salts, hydrates, solvates, tautomers, prodrugs, or optical isomers thereof.

27. The compound of claim 26 further defined as: 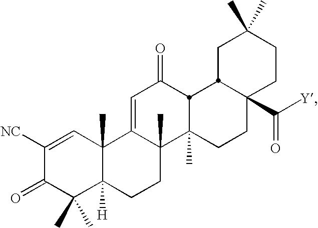
wherein Y′ is ethylamino or a heteroatom-substituted C₁-C₅-alkylamino having at least one fluorine atom; or
a pharmaceutically acceptable salt or hydrate thereof.

28. The compound of claim 27 further defined as: 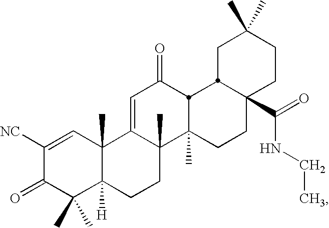
or a pharmaceutically acceptable salt or hydrate thereof.

29. The compound of claim 28, substantially free from optical isomers thereof.

30. The compound of claim 27, wherein Y′ is a heteroatom-substituted C₂-C₄-alkylamino having at least one fluorine atom.

31. The compound of claim 30, wherein the compound is further defined as: 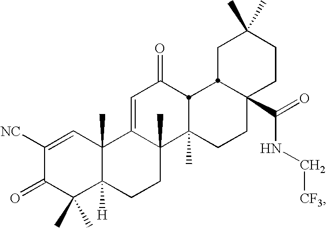
or a pharmaceutically acceptable salt or hydrate thereof.

32. The compound of claim 31, substantially free from optical isomers thereof.

33. A compound selected from the group consisting of:
(4aS,6aR,6bS,8aR,12aS,14aR,14bS)-11-cyano-N-ethyl-2,2,6a,6b,9,9,12a-heptamethyl-10,14-dioxo-1,2,3,4,4a,5,6,6a,6b,7,8,8a,9,10,12a,14,14a,14b-octadecahydropicene-4a-carboxamide; and
(4aS,6aR,6bS,8aR,12aS,14aR,14bS)-11-cyano-2,2,6a,6b,9,9,12a-heptamethyl-10,14-dioxo-N-(2,2,2-trifluoroethyl)-1,2,3,4,4a,5,6,6a,6b,7,8,8a,9,10,12a,14,14a, 14b-octadecahydropicene-4a-carboxamide.

34-48. (canceled)

49. A pharmaceutical composition comprising as an active ingredient a compound according to claim 26 and a pharmaceutically acceptable carrier.

50. The pharmaceutical composition of claim 49, wherein the composition is adapted for administration by a route selected from the group consisting of orally, intraadiposally, intraarterially, intraarticularly, intracranially, intradermally, intralesionally, intramuscularly, intranasally, intraocularally, intrapericardially, intraperitoneally, intrapleurally, intraprostaticaly, intrarectally, intrathecally, intratracheally, intratumorally, intraumbilically, intravaginally, intravenously, intravesicularlly, intravitreally, liposomally, locally, mucosally, orally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, via localized perfusion, bathing target cells directly, or any combination thereof.

51. The composition of claim 50, wherein the composition is formulated for oral delivery.

52. The composition of claim 51, wherein the composition is formulated as a hard or soft capsule, a tablet, a syrup, a suspension, a wafer, or an elixir.

53. The composition of claim 52, wherein the soft capsule is a gelatin capsule.

54. The composition of claim 51, further comprising a protective coating.

55. The composition of claim 51, further comprising an agent that delays absorption.

56. The composition of claim 51, further comprising an agent that enhances solubility or dispersibility.

57. The composition of claim 49, wherein the compound is dispersed in a liposome, an oil and water emulsion or a water and oil emulsion.

58. A therapeutic method comprising administering a pharmaceutically effective compound of claim 26 to a subject.

59. The method of claim 58, wherein the subject is a human.

60. The method of claim 58, further comprising identifying a subject in need of treatment.

61. A method of treating cancer in a subject, comprising administering to the subject a pharmaceutically effective amount of a compound of claim 26.

62. The method of claim 61, wherein the cancer is a carcinoma, sarcoma, lymphoma, leukemia, melanoma, mesothelioma, multiple myeloma, or seminoma.

63. The method of claim 61, wherein the cancer is of the bladder, blood, bone, brain, breast, central nervous system, colon, endometrium, esophagus, genitourinary tract, head, larynx, liver, lung, neck, ovary, pancreas, prostate, spleen, small intestine, large intestine, stomach, or testicle.

64. The method of claim 61, wherein the subject is a primate.

65. The method of claim 61, wherein the subject is a human.

66. The method of claim 61, further comprising identifying a subject in need of treatment.

67. The method of claim 66, wherein the subject has a family or patient history of cancer.

68. The method of claim 61, wherein the subject has symptoms of cancer.

69. The method of claim 61, wherein the compound is administered locally.

70. The method of claim 69, wherein the compound is administered by direct intratumoral injection or by injection into tumor vasculature.

71. The method of claim 61, wherein the compound is administered systemically.

72. The method of claim 71, wherein the compound is administered intravenously, intra-arterially, intramuscularly, intraperitoneally, subcutaneously or orally.

73. The method of claim 61, wherein the pharmaceutically effective amount is 0.1-1000 mg/kg.

74. The method of claim 73, wherein the pharmaceutically effective amount is administered in a single dose per day.

75. The method of claim 73, wherein the pharmaceutically effective amount is administered in two or more doses per day.

76. The method of claim 61, wherein the compound is administered by contacting a tumor cell during ex vivo purging.

77. The method of claim 61, wherein the method comprises:
a) inducing cytotoxicity in a tumor cell;
b) killing a tumor cell;
c) inducing apoptosis in a tumor cell;
d) inducing differentiation in a tumor cell; or
e) inhibiting growth in a tumor cell.

78. The method of claim 77, wherein the tumor cell is a leukemia cell.

79. The method of claim 77, wherein the tumor cell is a bladder cancer cell, a breast cancer cell, a lung cancer cell, a colon cancer cell, a prostate cancer cell, a liver cancer cell, a pancreatic cancer cell, a stomach cancer cell, a testicular cancer cell, a brain cancer cell, an ovarian cancer cell, a lymphatic cancer cell, a skin cancer cell, a brain cancer cell, a bone cancer cell, or a soft tissue cancer cell.

80. The method of claim 61, further comprising a treatment selected from the group consisting of administering a pharmaceutically effective amount of a second drug, radiotherapy, gene therapy, and surgery.

81. The method of claim 80, further comprising (1) contacting a tumor cell with the compound prior to contacting the tumor cell with the second drug, (2) contacting a tumor cell with the second drug prior to contacting the tumor cell with the compound, or (3) contacting a tumor cell with the compound and the second drug at the same time.

82. The method of claim 80, wherein the second drug is an antibiotic, anti-inflammatory, anti-neoplastic, anti-proliferative, anti-viral, immunomodulatory, or immunosuppressive.

83. The method of claim 80, wherein the second drug is an alkylating agent, androgen receptor modulator, cytoskeletal disruptor, estrogen receptor modulator, histone-deacetylase inhibitor, HMG-CoA reductase inhibitor, prenyl-protein transferase inhibitor, retinoid receptor modulator, topoisomerase inhibitor, or tyrosine kinase inhibitor.

84. The method of claim 80, wherein the second drug is 5-azacitidine, 5-fluorouracil, 9-cis-retinoic acid, actinomycin D, alitretinoin, all-trans-retinoic acid, annamycin, axitinib, belinostat, bevacizumab, bexarotene, bosutinib, busulfan, capecitabine, carboplatin, carmustine, CD437, cediranib, cetuximab, chlorambucil, cisplatin, cyclophosphamide, cytarabine, dacarbazine, dasatinib, daunorubicin, decitabine, docetaxel, dolastatin-10, doxifluridine, doxorubicin, doxorubicin, epirubicin, erlotinib, etoposide, etoposide, gefitinib, gemcitabine, gemtuzumab ozogamicin, hexamethylmelamine, idarubicin, ifosfamide, imatinib, irinotecan, isotretinoin, ixabepilone, lapatinib, LBH589, lomustine, mechlorethamine, melphalan, mercaptopurine, methotrexate, mitomycin, mitoxantrone, MS-275, neratinib, nilotinib, nitrosourea, oxaliplatin, paclitaxel, plicamycin, procarbazine, semaxanib, semustine, sodium butyrate, sodium phenylacetate, streptozotocin, suberoylanilide hydroxamic acid, sunitinib, tamoxifen, teniposide, thiopeta, tioguanine, topotecan, TRAIL, trastuzumab, tretinoin, trichostatin A, valproic acid, valrubicin, vandetanib, vinblastine, vincristine, vindesine, or vinorelbine.

85. A method of treating or preventing a disease with an inflammatory component in a subject, comprising administering to the subject a pharmaceutically effective amount of a compound of claim 26.

86. The method of claim 85, wherein the disease is lupus or rheumatoid arthritis.

87. The method of claim 85, wherein the disease is an inflammatory bowel disease.

88. The method of claim 87, wherein the inflammatory bowel disease is Crohn's disease or ulcerative colitis.

89. The method of claim 85, wherein the disease with an inflammatory component is a cardiovascular disease.

90. The method of claim 85, wherein the disease with an inflammatory component is diabetes.

91. The method of claim 90, wherein the diabetes is type 1 diabetes.

92. The method of claim 90, wherein the diabetes is type 2 diabetes.

93. The method of claim 90, wherein the pharmaceutically effective amount of the also effectively treats one or more complications associated with diabetes.

94. The method of claim 93, wherein the complications are selected from the group consisting of obesity, hypertension, atherosclerosis, coronary heart disease, stroke, peripheral vascular disease, hypertension, nephropathy, neuropathy, myonecrosis, retinopathy and metabolic syndrome (syndrome X).

95. The method of claim 85, wherein the disease with an inflammatory component is metabolic syndrome (syndrome X).

96. The method of claim 85, wherein the disease with an inflammatory component is a skin disease.

97. The method of claim 96, wherein the administration is topical or oral.

98. The method of claim 96, wherein the skin disease is psoriasis, acne, or atopic dermatitis.

99. A method of treating or preventing a cardiovascular disease in a subject, comprising administering to the subject a pharmaceutically effective amount of a compound of claim 26.

100. The method of claim 99, wherein the cardiovascular disease is atherosclerosis, cardiomyopathy, congenital heart disease, congestive heart failure, myocarditis, rheumatic heart disease, valve disease, coronary artery disease, endocarditis, or myocardial infarction.

101. The method of claim 99, further comprising administering a pharmaceutically effective amount of a second drug.

102. The method of claim 101, wherein the second drug is a cholesterol lowering drug, an anti-hyperlipidemic, a calcium channel blocker, an anti-hypertensive, or an HMG-CoA reductase inhibitor.

103. The method of claim 102, wherein the second drug is amlodipine, aspirin, ezetimibe, felodipine, lacidipine, lercanidipine, nicardipine, nifedipine, nimodipine, nisoldipine or nitrendipine.

104. The method of claim 102, wherein the second drug is atenolol, bucindolol, carvedilol, clonidine, doxazosin, indoramin, labetalol, methyldopa, metoprolol, nadolol, oxprenolol, phenoxybenzamine, phentolamine, pindolol, prazosin, propranolol, terazosin, timolol or tolazoline.

105. The method of claim 101, wherein the second drug is a statin.

106. The method of claim 105, wherein the statin is atorvastatin, cerivastatin, fluvastatin, lovastatin, mevastatin, pitavastatin, pravastatin, rosuvastatin or simvastatin.

107. A method of treating or preventing a neurodegenerative disease in a subject, comprising administering to the subject a pharmaceutically effective amount of a compound of claim 26.

108. The method of claim 107, wherein said neurodegenerative disease is selected from the group consisting of Parkinson's disease, Alzheimer's disease, multiple sclerosis (MS), Huntington's disease and amyotrophic lateral sclerosis.

109. The method of claim 108, wherein said neurodegenerative disease is Alzheimer's disease.

110-122. (canceled)

123. A method of treating or preventing a disorder characterized by overexpression of iNOS genes in a subject, comprising administering to the subject a pharmaceutically effective amount of a compound of claim 26.

124. A method of inhibiting IFN-γ-induced nitric oxide production in cells of a subject, comprising administering to said subject a pharmaceutically effective amount of a compound of claim 26.

125. A method of treating or preventing a disorder characterized by overexpression of COX-2 genes in a subject, comprising administering to the subject a pharmaceutically effective amount of compound of claim 26.

126. A method of treating renal/kidney disease (RKD) in a subject, comprising administering to the subject a pharmaceutically effective amount of a compound of claim 26.

127. The method of claim 126, wherein the RKD results from a toxic insult.

128. The method of claim 127, wherein the toxic insult results from an imaging agent or a drug.

129. The method of claim 128, wherein the drug is a chemotherapeutic.

130. The method of claim 126, wherein the RKD results from ischemia/reperfusion injury.

131. The method of claim 126, wherein the RKD results from diabetes or hypertension.

132. The method of claim 126, wherein the RKD results from an autoimmune disease.

133. The method of claim 126, wherein the RKD is chronic RKD.

134. The method of claim 126, wherein the RKD is acute RKD.

135. The method of claim 126, wherein the subject has undergone or is undergoing dialysis.

136. The method of claim 126, wherein the subject has undergone or is a candidate to undergo kidney transplant.

137. The method of claim 126, wherein the subject is a primate.

138. The method of claim 137, wherein the primate is a human.

139. (canceled)

140. A method for improving glomerular filtration rate or creatinine clearance in a subject, comprising administering to the subject a pharmaceutically effective amount of a compound of claim 26.

141. A kit comprising:
a compound of claim 26; and
instructions which comprise one or more forms of information selected from the group consisting of indicating a disease state for which the compound is to be administered, storage information for the compound, dosing information and instructions regarding how to administer the compound.

142. The kit according to claim 141, wherein the kit comprises the compound in a multiple dose form.

143. An article of manufacture comprising:
a compound of claim 26; and
packaging materials.

144. The article of manufacture according to claim 143, wherein the packaging materials comprise a container for housing the compound.

145. The article of manufacture according to claim 144, wherein the container comprises a label indicating one or more members of the group consisting of a disease state for which the compound is to be administered, storage information, dosing information and/or instructions regarding how to administer the compound.

146. The article of manufacture according to claim 143, wherein the article of manufacture comprises the compound in a multiple dose form.

147. A method for treating multiple sclerosis (MS) in a subject comprising, administering to said subject a pharmaceutically effective amount of a compound of the formula: 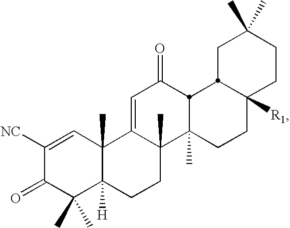
wherein R₁ is a heteroatom-substituted or heteroatom-unsubstituted C₁-C₁₅-acyl; or
a pharmaceutically acceptable salt or hydrate thereof.

148. The method of claim 147, wherein the MS is primary progressive.

149. The method of claim 147, wherein the MS is relapsing-remitting.

150. The method of claim 147, wherein the MS is secondary progressive.

151. The method of claim 147, wherein the MS is progressive relapsing.

152. The method of claim 147, wherein the treatment suppresses the demyelination of neurons in the subject's brain or spinal cord.

153. The method of claim 152, wherein the treatment suppresses inflammatory demyelination.

154. The method of claim 147, wherein the treatment suppresses the transection of neuron axons in the subject's brain or spinal cord.

155. The method of claim 147, wherein the treatment suppresses the transection of neurites in the subject's brain or spinal cord.

156. The method of claim 147, wherein the treatment suppresses neuronal apoptosis in the subject's brain or spinal cord.

157. The method of claim 147, wherein the treatment stimulates the remyelination of neuron axons in the subject's brain or spinal cord.

158. The method of claim 147, wherein the treatment restores lost function after an MS attack.

159. The method of claim 147, wherein the treatment prevents new MS attacks.

160. The method of claim 147, wherein the treatment prevents disability resulting from an MS attack.

161. The method of claim 147, wherein the subject is a primate.

162. The method of claim 161, wherein the primate is a human.

163. The method of claim 147, wherein the subject is a cow, horse, dog, cat, pig, mouse, rat or guinea pig.

164. The method of claim 147, wherein the compound is further defined as: 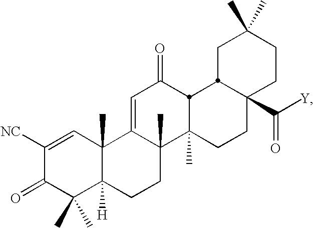
wherein Y is —H, hydroxy, amino, halo, or a heteroatom-substituted or heteroatom-unsubstituted C₁-C₁₄-alkoxy, C₂-C₁₄-alkenyloxy, C₂-C₁₄-alkynyloxy, C₁-C₁₄-aryloxy, C₂-C₁₄-aralkoxy, C₁-C₁₄-alkylamino, C₂-C₁₄-alkenylamino, C₂-C₁₄-alkynylamino, C₁-C₁₄-arylamino, or C₂-C₁₄-aralkylamino; or
a pharmaceutically acceptable salt or hydrate thereof.

165. The method of claim 164, wherein Y is a heteroatom-substituted C₁-C₄-alkylamino.

166. The method of claim 165, wherein the compound is further defined as: 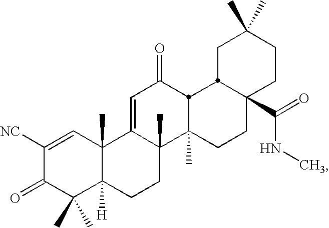
substantially free from other optical isomers.

167. The method of claim 165, wherein Y is a heteroatom-substituted or heteroatom-unsubstituted C₂-C₄-alkylamino.

168. The method of claim 167, wherein the compound is further defined as: 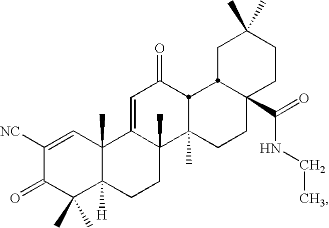
substantially free from other optical isomers.

169. The method of claim 167, wherein the compound is further defined as: 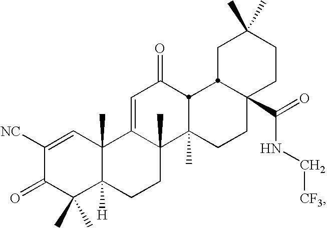
substantially free from other optical isomers.

170. The method of claim 164, wherein Y is a heteroatom-substituted or heteroatom-unsubstituted C₁-C₄-alkoxy.

171. The method of claim 170, wherein the compound is further defined as: 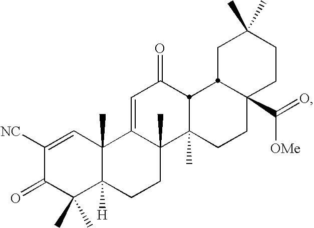
substantially free from other optical isomers.

172. The method of claim 164, wherein the compound is further defined as: 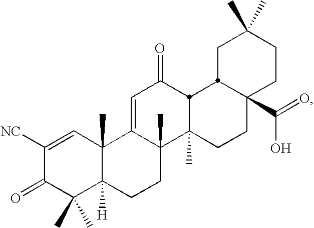
substantially free from other optical isomers.

173. A method for treating multiple sclerosis (MS) in a subject comprising, administering to said subject:
a) a first amount of a first compound having the structure: 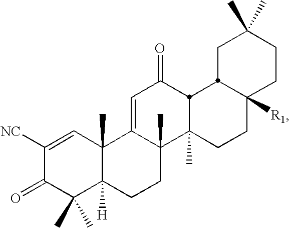
wherein R₁ is a heteroatom-substituted or heteroatom-unsubstituted C₁-C₁₅-acyl; or
a pharmaceutically acceptable salt or hydrate thereof; and
b) a second amount of a compound selected from the group consisting of interferon β-1 a, interferon β-1 b, glatiramer acetate, mitoxantrone, natalizumab, uric acid, and methylprednisolone;
wherein the combined first and second amounts are effective to treat the MS.

174. A method for treating a spinal cord injury in a subject comprising, administering to said subject a pharmaceutically effective amount of a compound having the structure: 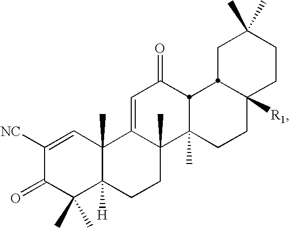
wherein R₁ is a heteroatom-substituted or heteroatom-unsubstituted C₁-C₁₅-acyl;
or
a pharmaceutically acceptable salt or hydrate thereof.

175. The method of claim 174, wherein the treatment restores lost function related to the spinal cord injury.

176. The method of claim 174, wherein the treatment prevents a disability related to the spinal cord injury.

177. The method of claim 174, wherein the subject is a primate.

178. The method of claim 177, wherein the primate is a human.

179. The method of claim 174, wherein the compound is further defined as 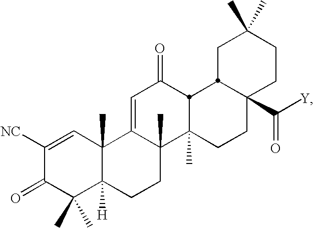
wherein Y is —H, hydroxy, amino, halo, or a heteroatom-substituted or heteroatom-unsubstituted C₁-C₁₄-alkoxy, C₂-C₁₄-alkenyloxy, C₂-C₁₄-alkynyloxy, C₁-C₁₄-aryloxy, C₂-C₁₄-aralkoxy, C₁-C₁₄-alkylamino, C₂-C₁₄-alkenylamino, C₂-C₁₄-alkynylamino, C₁-C₁₄-arylamino, or C₂-C₁₄-aralkylamino; or
a pharmaceutically acceptable salt or hydrate thereof.

180. The method of claim 179, wherein Y is a heteroatom-substituted C₁-C₄-alkylamino.

181. The method of claim 180, wherein the compound is further defined as: 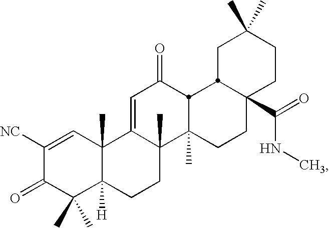
substantially free from other optical isomers.

182. The method of claim 180, wherein Y is a heteroatom-substituted or heteroatom-unsubstituted C₂-C₄-alkylamino.

183. The method of claim 182, wherein the compound is further defined as: 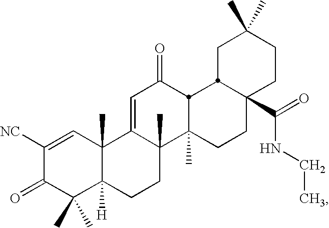
substantially free from other optical isomers.

184. The method of claim 182, wherein the compound is further defined as: 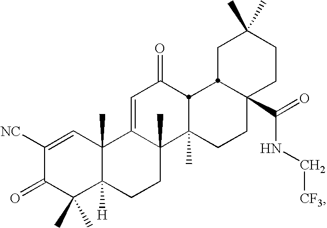
substantially free from other optical isomers.

185. The method of claim 179, wherein Y is a heteroatom-substituted or heteroatom-unsubstituted C₁-C₄-alkoxy.

186. The method of claim 185, wherein the compound is further defined as: 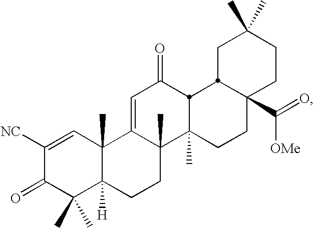
substantially free from other optical isomers.

187. The method of claim 179, wherein the compound is further defined as: 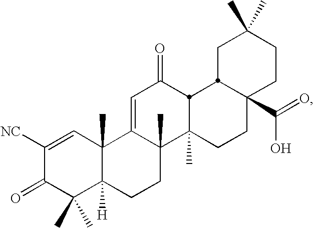
substantially free from other optical isomers.
